SEQUENCE LISTING (3 sequences)

SEQ ID NO: 1 - DNA, 13 bp, unidentified
  source 1..13: mol_type = unassigned DNA
gggaagggcgatc

SEQ ID NO: 2 - DNA, 12 bp, unidentified
  source 1..12: mol_type = unassigned DNA
gggaagggcgat

SEQ ID NO: 3 - DNA, 10 bp, unidentified
  source 1..10: mol_type = unassigned DNA
gggaagggcg

(19) United States
(12) Patent Application Publication
(10) Pub. No.: US 2024/0412814 A1
(43) Pub. Date: Dec. 12, 2024

(54) METHODS FOR NEOPLASIA DETECTION FROM CELL FREE DNA

(71) Applicants: The Broad Institute, Inc., Cambridge, MA (US); President and Fellows of Harvard College, Cambridge, MA (US); The General Hospital Corporation, Boston, MA (US)

(72) Inventors: Gad GETZ, Boston, MA (US); Ziao LIN, Cambridge, MA (US); Donald STEWART, Cambridge, MA (US)

(73) Assignees: The Broad Institute, Inc., Cambridge, MA (US); President and Fellows of Harvard College, Cambridge, MA (US); The General Hospital Corporation, Boston, MA (US)

(21) Appl. No.: 18/813,575

(22) Filed: Aug. 23, 2024

Related U.S. Application Data
(63) Continuation of application No. PCT/US2023/063139, filed on Feb. 23, 2023.
(60) Provisional application No. 63/313,663, filed on Feb. 24, 2022.

(51) Int. Cl. G16B 20/10; G16B 30/00

(52) U.S. Cl. CPC G16B 20/10; G16B 30/00

(57) ABSTRACT

The disclosure features compositions and methods that are useful for determining the fraction of tumor-derived DNA (tumor fraction; TF) in cell free DNA (cfDNA). The methods involve calculating the fraction of tumor-derived DNA in the cfDNA using a combination of copy number alteration data and fragment length distribution data.

### CROSS-REFERENCE TO RELATED APPLICATION

This application is a continuation of International Application No. PCT/US2023/063139, which claims priority to and the benefit of U.S. Provisional Application No. 63/313,663, filed Feb. 24, 2022, the entire contents of which are incorporated herein by reference.

### STATEMENT OF RIGHTS TO INVENTIONS MADE UNDER FEDERALLY SPONSORED RESEARCH

This invention was made with government support under Grant No. CA264024 awarded by the National Institutes of Health. The government has certain rights in the invention.

### SEQUENCE LISTING

The present application contains a Sequence Listing which has been submitted electronically in XML format following conversion from the originally filed TXT format.

The content of the electronic XML Sequence Listing, (Date of creation: Mar. 29, 2023; Size: 3,673 bytes: Name: 167741-047001PCT_SL.xml), is herein incorporated by reference in its entirety.

### BACKGROUND OF THE INVENTION

Early neoplasia (e.g., a cancer or tumor) diagnosis and rapid therapeutic intervention are critical for decreasing cancer morbidity and mortality. However, because of low sensitivity and specificity, as well as lack of general applicability across various cancer types, existing protein-based biomarkers from blood are not generally feasible for pan-cancer screening. Moreover, cancer detection tools that leverage tumor fraction (TF) estimation would be most powerful in the clinic if used not only for detection of cancer at early stages, but also for early detection of resistant clones that may develop on treatment, providing opportunities for additional therapeutic intervention to stem the tide of full-blown resistance.

In view of the foregoing, there is an urgent unmet need for methods for detecting and characterizing a neoplasia in a subject.

### SUMMARY OF THE INVENTION

As described below, the present disclosure features compositions and methods that are useful for characterizing a neoplasia in a subject. The methods disclosed herein generally involve determining the fraction of tumor-derived DNA (tumor fraction: TF) in cell free DNA (cfDNA) and calculating the fraction of tumor-derived DNA in the cfDNA using a combination of copy number alteration data and fragment length distribution data.

In one aspect, the disclosure features a method for characterizing DNA in a biological sample from a subject having or suspected of having a neoplasia. The method involves (a) sequencing cell free DNA (cfDNA) derived from a biological sample to obtain sequence data. The method also involves (b) analyzing the sequence data to determine a copy number profile and DNA fragment length abundance profile. The method further involves (c) calculating a tumor fraction in the cfDNA based upon the copy number profile and the fragment length abundance profile, thereby characterizing the DNA in the biological sample.

In another aspect, the disclosure features a method for characterizing DNA in a biological sample from a subject having or suspected of having a neoplasia. The method involves (a) sequencing cell free DNA (cfDNA) derived from a biological sample to obtain sequence data. The method also involves (b) analyzing the sequence data to calculate a copy number profile and DNA fragment length abundance profile. The fragment length abundance profile has a signal-to-noise ratio (SNR) of at least 2 and an absolute correlation coefficient of at least 0.1 with log₂ transformed copy ratios associated with a neoplasia. The method further involves (c) using a probabilistic model combining the copy number profile and the DNA fragment length abundance profile to calculate tumor fraction in the cfDNA, thereby characterizing the DNA in the biological sample.

In another aspect, the disclosure features a method for identifying the presence of a neoplasia in a biological sample from a subject having or suspected of having a neoplasia. The method involves (a) sequencing cell free DNA (cfDNA) derived from a biological sample derived from the subject to obtain sequence data. The method also involves (b) analyzing the sequence data to determine a copy number profile and DNA fragment length abundance profile. The method further involves (c) calculating a tumor fraction in the cfDNA based upon the copy number profile and the fragment length abundance profile. The method identifies the presence or absence of a neoplasia in the biological sample.

In another aspect, the disclosure features a method for detecting resistance to therapy in a subject being treated for a neoplasia. The method involves (a) sequencing cell free DNA (cfDNA) derived from two or more biological samples derived from the subject to obtain sequence data. The biological samples are obtained at one or more time points during the course of treatment. The method also involves (b) analyzing the sequence data to determine a copy number profile and DNA fragment length abundance profile. The method further involves (c) calculating a tumor fraction in the cfDNA based upon the copy number profile and the fragment length abundance profile. A significant increase in tumor fraction over time and/or a tumor fraction above a threshold value detects resistance.

In another aspect, the disclosure features a method for monitoring therapy in a subject being treated for a neoplasia. The method involves (a) sequencing cell free DNA (cfDNA) derived from two or more biological samples derived from the subject to obtain sequence data. The biological samples are obtained at one or more time points during the course of treatment. The method also involves (b) analyzing the sequence data to determine a copy number profile and DNA fragment length abundance profile. The method further involves (c) calculating a tumor fraction in the cfDNA based upon the copy number profile and the fragment length abundance profile, thereby monitoring the therapy.

In another aspect, the disclosure features a method for characterizing the disease state of a subject. The method involves (a) sequencing cell free DNA (cfDNA) derived from a biological sample to obtain sequence data. The method also involves (b) determining in the sequence data the DNA fragment length abundance profile for DNA fragments with lengths of from about 261 to about 310 bp. The method further involves (c) using a probabilistic model to calculate a tumor fraction in the cfDNA based upon the DNA fragment length abundance profile. A non-zero tumor fraction indicates that the subject has a neoplasia.

In another aspect, the disclosure features a method for characterizing the disease state of a subject. The method involves (a) sequencing cell free DNA (cfDNA) derived from a biological sample to obtain sequence data. The method also involves (b) determining in the sequence data the DNA fragment length abundance profile for DNA fragments with lengths of from about 261 to about 310 bp. The method also involves (c) using a probabilistic model to calculate a tumor fraction in the cfDNA based upon the DNA fragment length abundance profile. A non-zero tumor fraction indicates that the subject has a neoplasia.

In another aspect, the disclosure features a computer-implemented method. The method involves receiving sequencing data from a plurality of cfDNA obtained from a plurality of biological samples. The method also involves defining, for a plurality of cfDNA present in a biological sample, a copy number profile and a fragment length abundance profile. The copy number profile comprises a copy ratio of a plurality of somatic copy number alterations (SCNA). The fragment length abundance profile contains one or more of a plurality of aligned reads and an associated fragment length distribution for non-overlapping bins of the sequencing data. The method also involves determining whether a Signal-to-noise Ratio (SNR) across the fragment length abundance profile and a correlation coefficient of the copy ratio and a fraction of fragments associated with a neoplasia satisfy one or more criteria. The method further involves calculating, based on at least one of the fragment length abundance profile for which the SNR satisfies the one or more criteria and the copy ratio and the fraction of fragments for which the correlation coefficient satisfies the one or more criteria, a tumor fraction (TF) of the biological sample.

In another aspect, the disclosure features a computer-implemented method. The method involves sequencing polynucleotide data from a plurality of biological samples. The method further involves identifying a copy ratio of a plurality of somatic copy number alterations (SCNA) and an associated fragment length distribution for non-overlapping bins of the sequencing data. The method also involves determining whether a Signal-to-noise Ratio (SNR) across the fragment length distribution and a correlation coefficient of the copy ratio and the fragment length distribution associated with a neoplasia satisfy one or more criteria. The method also involves calculating, based on at least one of a size of a genomic bin and a number of genomic bins of the sequencing data, a tumor fraction (TF) profile of the biological sample. The method further involves determining, based on the fragment length distribution for which the SNR satisfies the one or more criteria, a copy ratio for which the correlation coefficient satisfies the one or more criteria, and the TF profile, whether the polynucleotide data came from cancer cells.

In any of the above aspects, or embodiments thereof, the TF profile is calculated based on one or more of a total copy number of a genomic bin in the cancer cells, a length of the genomic bin, a total number of genomic bins, a fraction of fragments in healthy donors inferred from a panel of normals (PoN), and a fraction of cancer cells-derived fragments inferred from cfDNA samples with high tumor fraction.

In any of the above aspects, or embodiments thereof, the DNA fragment length abundance profile has a signal-to-noise ratio (SNR) of at least 2 and an absolute correlation coefficient of at least 0.1 with log₂ transformed copy ratios associated with a neoplasia.

In any of the above aspects, or embodiments thereof, the biological sample contains a liquid or solid sample. In any of the above aspects, or embodiments thereof, the biological sample contains a bodily fluid. In embodiments, the bodily fluid contains ascites, blood, plasma, pleural fluid, serum, cerebrospinal fluid, phlegm, saliva, urine, semen, stool, prostate fluid, breast milk, or tears. In embodiments, the solid sample is a tissue sample. In embodiments, the tissue sample is a biopsy.

In any of the above aspects, or embodiments thereof, the subject is a mammal. In any of the above aspects, or embodiments thereof, the subject is a human.

In any of the above aspects, or embodiments thereof, the fragment length abundance profile is calculated for fragment lengths between about 100 and about 500 base pairs. In any of the above aspects, or embodiments thereof, the fragment-length abundance profile is calculated for fragment lengths between about 100 and about 400 base pairs. In any of the above aspects, or embodiments thereof, the fragment-length abundance profile is calculated for fragment lengths between about 200 and about 400 base pairs. In any of the above aspects, or embodiments thereof, the fragment-length abundance profile is calculated for fragment lengths between about 261 and about 310 base pairs.

In any of the above aspects, or embodiments thereof, the SNR is calculated across contiguous fragment-length bins within a range of fragment lengths for which the fragment length abundance profile is calculated. In any of the above aspects, or embodiments thereof, the SNR is calculated as SNR_{i,j}, where i is a cell free DNA sample, j is a bin of fragment lengths, and SNR_{i,j} is the fraction of those fragments j in sample i minus the average fraction in a panel of healthy donors, and then divided by the standard deviation of the fraction in the panel of healthy donors. In any of the above aspects, or embodiments thereof, the SNR is a maximum SNR calculated in a bin within a fragment-length range for which the DNA fragment length abundance profile is calculated. In embodiments, the bin is 5 bp, 10 bp, 15 bp, or 20 bp in size. In any of the above aspects, or embodiments thereof, the SNR is calculated as SNRᵣ=(F^{t}ᵣ−F̅^{H}ᵣ)/std(F^{H}ᵣ), where F^{t}ᵣ represents DNA fragment length bin r in biological sample t, and \hat{F}^{H}ᵣ represents the average over a healthy panel of normals of the fraction of DNA fragments in fragment length bin r. In any of the above aspects, or embodiments thereof, the SNR is at least about 3 or 4.

In any of the above aspects, or embodiments thereof, the correlation coefficient is a Spearman Correlation Coefficient. In any of the above aspects, or embodiments thereof, the absolute correlation coefficient is at least about 0.2 or 0.3. In any of the above aspects, or embodiments thereof, the correlation coefficient is calculated between the log_2-transformed copy ratio and the fraction of fragments in DNA fragment length bin r across the top 10% of those genomic segments with the highest copy ratios corresponding to amplifications and the bottom 10% of those genomic segments with copy ratios corresponding to deletions.

In any of the above aspects, or embodiments thereof, the tumor fraction in the cfDNA is calculated using a Bayesian model. In any of the above aspects, or embodiments thereof, the probabilistic model is a Bayesian model. In embodiments, the Bayesian model is an interpretable Bayesian graphical model.

In any of the above aspects, or embodiments thereof, the tumor fraction is less than about 0.03. In any of the above aspects, or embodiments thereof, the tumor fraction is from about 1e-4 to about 0.03. In any of the above aspects, or embodiments thereof, the tumor fraction is from about 5e-3 to about 0.15. In any of the above aspects, or embodiments thereof, the tumor fraction is between about 1e-5 and about 0.1. In any of the above aspects, or embodiments thereof, the tumor fraction is less than 0.01.

In any of the above aspects, or embodiments thereof, the method further involves comparing the copy number profile and the fragment length abundance profile to a matched normal sample(s). In embodiments, the matched normal sample is from a healthy subject. In embodiments, the healthy subject is the same subject from which the biological sample was collected.

In any of the above aspects, or embodiments thereof, the neoplasia is selected from one or more of the following: bile duct cancer, bladder cancer, breast cancer, colon cancer, head-and-neck cancer, liver cancer, lung cancer, intrahepatic bile duct cancer, prostate, ovarian cancer, skin cancer, stomach cancer, thyroid, and chronic lymphocytic leukemia (Richter's transformation).

In any of the above aspects, or embodiments thereof, the sequencing coverage is less than about 5×. In any of the above aspects, or embodiments thereof, the sequencing coverage is about 0.1× or 0.2×.

In any of the above aspects, or embodiments thereof, the tumor fraction is determined with a mean absolute error of from about 0% to about 20%. In any of the above aspects, or embodiments thereof, the tumor fraction is determined with a mean absolute error of from about 4.5% to about 11%.

In any of the above aspects, or embodiments thereof, the sequencing is next generation sequencing. In any of the above aspects, or embodiments thereof, the sequencing is ultra low-pass whole genome sequencing.

In any of the above aspects, or embodiments thereof, the calculating is done on a computer system.

In any of the above aspects, or embodiments thereof, the threshold value is at least about 5%. In any of the above aspects, or embodiments thereof, the threshold value is at least about 10%. In any of the above aspects, or embodiments thereof, the increase is at least a 1% increase. In any of the above aspects, or embodiments thereof, the increase is at least a 2-fold increase.

In any of the above aspects, or embodiments thereof, the method further involves collecting biological samples from the subject about once per day, every 3 days, every 1 week, 2 weeks, 3 weeks, or month and determining tumor fraction in the cfDNA of each biological sample. In any of the above aspects, or embodiments thereof, the method further involves collecting biological samples from the subject about once every 1 year and determining tumor fraction in the cfDNA of each biological sample.

In any of the above aspects, or embodiments thereof, the therapy is chemotherapy, radiation, or immunotherapy.

In any of the above aspects, or embodiments thereof, the copy number profile and/or the DNA fragment length abundance profile is calculated over 1, 2, 3, 4, 5, or all genomic loci represented in the sequence data.

The disclosure provides compositions and methods that are useful for determining the fraction of tumor-derived DNA (tumor fraction: TF) in cell free DNA (cfDNA). Compositions and articles defined by the disclosure were isolated or otherwise manufactured in connection with the examples provided below. Other features and advantages of the disclosure will be apparent from the detailed description, and from the claims.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994): The Cambridge Dictionary of Science and Technology (Walker ed., 1988): The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof.

As used herein, the term “algorithm” refers to any formula, model, mathematical equation, algorithmic, analytical, or programmed process, or statistical technique or classification analysis that takes one or more inputs or parameters, whether continuous or categorical, and calculates an output value, index, index value or score. Examples of algorithms include but are not limited to ratios, sums, regression operators such as exponents or coefficients, biomarker value transformations and normalizations (including, without limitation, normalization schemes that are based on clinical parameters such as age, gender, ethnicity, etc.), rules and guidelines, statistical classification models, statistical weights, and neural networks trained on populations or datasets. Also, of use in the context of TuFEst as described herein are Bayesian models useful inferring an underlying tumor fraction and/or total copy number profile in circulating cell free DNA (cfDNA).

By “ameliorate” is meant decrease, suppress, attenuate, diminish, arrest, or stabilize the development or progression of a disease.

By “alteration” is meant a change in the structure, expression levels or activity of a gene or polypeptide as detected by standard art known methods such as those described herein. The alteration can be an increase or a decrease. As used herein, an alteration includes a 10% change in expression levels, preferably a 25% change, more preferably a 40% change, and most preferably a 50% or greater change in expression levels. In embodiments, the change is an amino acid or nucleobase sequence alteration.

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “bin” is meant a set of members. In one embodiment, a bin described herein comprises a set of polynucleotide fragments of particular lengths. A bin can be specified by the difference between a maximum size fragment and a minimum size fragment falling within the bin. For example, a bin that is 10 bp in size represents a range of polynucleotide fragment lengths within a range of fragment lengths spanning 10 bp. More particularly, in one example a bin of 10 bp can correspond to those DNA fragments with a size of from about 261 bp to about 270 bp. In embodiments, a bin corresponds to a set of polynucleotide fragment lengths falling within a larger fragment length range.

The term “cancer” refers to a malignant neoplasm. It is also contemplated within the scope of the disclosure that the techniques herein may be applied to detect and/or monitor a cancer in a subject.

In this disclosure, “comprises,” “comprising.” “containing” and “having” and the like can have the meaning ascribed to them in U.S. Patent law and can mean “includes,” “including,” and the like: “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. Determination of statistical significance is within the ability of those skilled in the art, e.g., the number of standard deviations from the mean that constitute a positive result. In embodiments, a reference is a subject or a sample from a subject that does not have a cancer or a subject prior to a change in a treatment or administration of a drug or treatment. In embodiments, the reference is a matched normal sample, where in some instances the matched normal sample is a sample from a healthy subject and/or a subject that does not have a cancer (e.g., a subject prior to being diagnosed with a cancer or neoplasm).

By “copy number profile” is meant a set of copy number alterations present in a biological sample relative to a reference. In embodiments, the biological sample comprises cell free DNA. In some instances, the reference is a reference sequence that is a genome of a healthy subject or the sequence of cell free DNA from a healthy subject or panel of healthy subjects.

As used herein, the term “coverage” refers to the number of sequence reads that align to a specific locus in a reference sequence. In embodiments, the reference sequence is a reference genome. For example, with regard to the terminal base of the following reference sequence, because there is only one sample base aligned at this locus (the bold cytosine in Read 2), there is 1× coverage of the reference sequence at this locus. At the 5′ end, there is 3× coverage of the reference sequence at the 5′ terminus guanine.
Reference Sequence: 5′ GGGAAGGGCGATC 3′ (SEQ ID NO: 1)
Read 1 GGGAAGGGCGAT (SEQ ID NO: 2)
Read 2 GGGAAGGGCGATC (SEQ ID NO: 1)
Read 3 GGGAAGGGCG (SEQ ID NO: 3)

When a genome is sequenced, there will be a large number of nucleotides sequenced. If an individual genome is sequenced only once, there will be a significant number of sequencing errors. To increase the sequencing accuracy, an individual genome will need to be sequenced a large number of times. The average coverage for a whole genome can be calculated from the length of the original genome (G), the number of reads (N), and the average read length (L) as N×L/G. In another example, a hypothetical genome with 2,000 base pairs reconstructed from 8 reads with an average length of 500 nucleotides will have 2× redundancy. This parameter also enables one to estimate other quantities, such as the percentage of the genome covered by reads (sometimes also called breadth of coverage). At a coverage of 0.1×, only 10% of a reference sequence is covered by sequence reads. In embodiments, a sample polynucleotide is sequenced to a coverage of about, at least about, and/or no more than about 1e-8, 1e-7, 1e-6, 1e-5, 1e-4, 1e-3, 1e-2, 0.05×, 0.1×, 0.2×, 0.3×, 0.4×, 0.5×, 1×, 2×, 3×, 4×, 5×, 7×, 8×, 9×, 10×, 20×, 30×, 40×, 50×, 60×, 70×, 90×, 100×, or more.

By “ultra-low coverage” is meant a coverage of less than at least 5×. In some instances, ultra-low coverage is a coverage of less than 0.5×, 0.2×, or 0.1×.

“Detect” refers to identifying the presence, absence, or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. In embodiments, the disease is a neoplasia.

By “disease state” is meant the presence, absence, and/or severity of a disease.

By “DNA fragment length abundance profile” is meant a set of DNA fragment length abundance measurements at one or more genetic loci. In embodiments, the DNA fragment length abundance profile is determined for DNA fragments falling within a predetermined length-range (e.g., from about 261 bp to about 310 bp) at about, at least about, or no more than about 1, 2, 3, 4, 5, 6, 7, 8, 9, 10, 100, 1,000, 10,000, 100,000, 1,000,000, or all genomic loci for a sample.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications, or dosages. A therapeutically effective amount of a therapeutic compound (i.e., an effective dosage) depends on the therapeutic compounds selected. The compositions can be administered from one or more times per day to one or more times per week: including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “increase” is meant to alter positively by at least 5% relative to a reference. An increase may be by 5%, 10%, 25%, 30%, 50%, 75%, or even by 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid that is free of the genes which, in the naturally-occurring genome of the organism from which the nucleic acid molecule of the disclosure is derived, flank the gene. The term therefore includes, for example, a recombinant DNA that is incorporated into a vector: into an autonomously replicating plasmid or virus: or into the genomic DNA of a prokaryote or eukaryote: or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the disclosure. An isolated polypeptide of the disclosure may be obtained, for example, by extraction from a natural source, by expression of a recombinant nucleic acid encoding such a polypeptide: or by chemically synthesizing the protein. Purity can be measured by any appropriate method, for example, column chromatography, polyacrylamide gel electrophoresis, or by HPLC analysis.

By “liquid biopsy” is meant the isolation and analysis of tumor derived material from blood or other bodily fluids. In embodiments, the material contains DNA, RNA, and/or intact cells. In some cases, the material does not contain intact cells. In some instances, the tumor-derived material is cell free DNA (cfDNA).

By “marker” is meant any protein or polynucleotide having an alteration in expression level or activity that is associated with a developmental state, condition, disease, or disorder.

By “neoplasia” is meant a disease or disorder characterized by excess proliferation or reduced apoptosis. In embodiments, a neoplasia is a cancer or tumor. Illustrative neoplasms include breast cancer, esophageal cancer, head-and-neck cancer, pancreatic cancer, skin cancer, colorectal cancer, hepatocellular cancer, bladder cancer, bile duct cancer, luminal and non-luminal bladder cancer, basal bladder cancer, muscle-invasive bladder cancer, and non-muscle-invasive bladder cancer, pancreatic cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, liver cancer, cervical cancer, uterine cancer, testicular cancer, lung carcinoma, small cell lung carcinoma, bladder carcinoma, epithelial carcinoma, glioma, glioblastoma multiforme, astrocytoma, medulloblastoma, craniopharyngioma, ependymoma, pincaloma, hemangioblastoma, acoustic neuroma, oligodenroglioma, schwannoma, meningioma, melanoma, neuroblastoma, and retinoblastoma). In embodiments, the neoplasia may be colon adenocarcinoma (COAD), stomach adenocarcinoma (STAD), stomach cancer, and uterine corpus endometrial carcinoma (UCEC). In embodiments, the neoplasia may be a liquid tumor such as, for example, leukemia or lymphoma. In embodiments, the cancer is a bile duct, bladder, breast, colon, head-and-neck, liver, lung, and/or intrahepatic bile ducts cancer, lung, ovarian, prostate, skin, thyroid, or stomach cancer, or a chronic lymphocytic leukemia (Richter's transformation).

As used herein, the term “next-generation sequencing (NGS)” refers to a variety of high-throughput sequencing technologies that parallelize the sequencing process, producing thousands or millions of sequence reads at once. NGS parallelization of sequencing reactions can generate hundreds of megabases to gigabases of nucleotide sequence reads in a single instrument run. Unlike conventional sequencing techniques, such as Sanger sequencing, which typically report the average genotype of an aggregate collection of molecules, NGS technologies typically digitally tabulate the sequence of numerous individual DNA fragments (sequence reads discussed in detail below), such that low frequency variants (e.g., variants present at less than about 10%, 5% or 1% frequency in a heterogeneous population of nucleic acid molecules) can be detected. The term “massively parallel” can also be used to refer to the simultaneous generation of sequence information from many different template molecules by NGS. NGS sequencing platforms include, but are not limited to, the following: Massively Parallel Signature Sequencing (Lynx Therapeutics): 454 pyro-sequencing (454 Life Sciences/Roche Diagnostics): solid-phase, reversible dye-terminator sequencing (Solexa/Illumina): SOLID technology (Applied Biosystems): Ion semiconductor sequencing (ion Torrent); and DNA nanoball sequencing (Complete Genomics). Descriptions of certain NGS platforms can be found in the following: Shendure, et al., “Next-generation DNA sequencing.” Nature, 2008, vol. 26, No. 10, 135-1 145; Mardis, “The impact of next-generation sequencing technology on genetics,” Trends in Genetics, 2007, vol. 24, No. 3, pp. 133-141: Su, et al., “Next-generation sequencing and its applications in molecular diagnostics” Expert Rev Mol Diagn, 2011, 11 (3): 333-43; and Zhang et al., “The impact of next-generation sequencing on genomics,” J Genet Genomics, 201, 38 (3): 95-109.

As used herein, “obtaining” as in “obtaining an agent” includes synthesizing, purchasing, or otherwise acquiring the agent.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects, the disclosure embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M. I. L. V: (b) F. Y, W: (c) K. R. H: (d) A, G: (c) S, T: (f) Q, N; and (g) E, D. In various embodiments, conservative amino acid substitutions can be made to the amino acid sequence of the proteins and polypeptides disclosed herein.

By “probabilistic model” is meant a statistical model used to define relationships between variables based upon one or more probability distributions. A non-limiting example of a probabilistic model is a Bayesian model, such as an interpretable Bayesian graphical model.

By “reduce” is meant to alter negatively by at least 5% relative to a reference. A reduction may be by 5%, 10%, 25%, 30%, 50%, 75%, or even by 100%.

A “reference sequence” is a defined sequence used as a basis for sequence comparison. A reference sequence may be a subset of or the entirety of a specified sequence: for example, a segment of a full-length cDNA or gene sequence, or the complete cDNA or gene sequence. For polypeptides, the length of the reference polypeptide sequence will generally be at least about 10 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween. In embodiments a “reference sequence” is the meant a single genome from a healthy donor or a representative genome that reflects input from a set of genomes. In some cases, a “reference sequence” is a sequence of a polynucleotide sample (e.g., a cfDNA sample) collected from a healthy subject or from a panel of healthy subjects. In embodiments, the “reference sequence” is a collection of polynucleotide sequences corresponding to a panel of healthy subjects.

By “signal to noise ratio (SNR)” is meant the level of a desired signal relative to the level of undesired background variation.

By “specifically binds” is meant a compound or antibody that recognizes and binds a polypeptide of the disclosure, but which does not substantially recognize and bind other molecules in a sample, for example, a biological sample, which naturally includes a polypeptide of the disclosure.

Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C. and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977): Grunstein and Hogness (Proc. Natl. Acad. Sci., USA 72:3961, 1975): Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid level to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine: valine, isoleucine, leucine: aspartic acid, glutamic acid, asparagine, glutamine: serine, threonine: lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant an animal. The animal can be a mammal. The mammal can be a human or non-human mammal, such as a bovine, equine, canine, ovine, rodent, or feline.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treatment,” “treating,” “treat” and the like, refer to obtaining a desired pharmacologic and/or physiologic effect. “Treatment,” as used herein, covers any treatment of a disease or condition in a mammal, particularly in a human, and includes inhibiting the disease (e.g., arresting its development) and/or relieving the disease (e.g., causing regression of the disease). In embodiments, treatment ameliorates at least one symptom of a neoplasia. For example, a treatment can result in a reduction in tumor size, tumor growth, cancer cell number, cancer cell growth, or metastasis or risk of metastasis. “Tumor derived DNA” means DNA that is derived from a cancer cell rather than a healthy control cell. Tumor derived DNA often includes structural changes that are indicative of cancer. Such structural changes may be at the level of the chromosome, which includes aneuploidy (abnormal number of chromosomes), duplications, deletions, or inversions, or alterations in sequence.

The term “tumor fraction” means the portion of DNA in a sample derived from or predicted to be derived from neoplastic cells. In embodiments, the DNA is cell free DNA (cfDNA).

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1G provide plots, a box plot, and charts demonstrating that abundance of specific cfDNA fragment lengths could distinguish donors with cancer from healthy donors. FIG. 1A, provides a plot showing fragment length distribution across 1-500 bp (normalized against the total number of fragments ≤1000 bp) in high tumor fraction (high-TF) cases (4th quartile. TF ≥0.44, N=49), low-TF (2nd quartile, 0.18≤TF≤0.28, N=51) breast cancer cfDNA samples and cfDNA samples from healthy donors (N=72). TF of each cancer cfDNA sample was assessed by ABSOLUTE using WES data (˜150×). Inset of FIG. 1A: distribution of 261-310 bp fragments. FIG. 1B also provides a box plot showing Fraction of 261-310 bp fragments in high-TF (TF≥0.44, N=49), low-TF (0.18≤TF≤0.28, N=51) breast cancer cfDNA samples and cfDNA samples from healthy donors (HD) (N=72). FIG. 1C, provides a plot of signal-to-noise ratios (SNR) of fragments between 50 and 500 bp (10 bp bins, x-axis) in breast cancer cfDNA samples (N=194). Grey shades—probability density of SNR across the breast cancer cohort. Dark grey marker-mean±standard error of the mean of the SNR across the cohort. The vertical dashed grey lines represent the lower (261 bp) and upper (310 bp) limit of the selected bin. FIG. 1D, provides a chart summarizing the mean and 95% confidence intervals of 5 bins in the selected bin. FIG. 1E, provides a plot showing the Spearman correlation coefficient (p) between the relative cancer concentration (defined as log₂(copy ratio)) and the fragment length relative abundance (50-500 bp. 10 bp bins) in genomic regions with extreme copy ratio (≥95th percentile or ≤5th percentile) in healthy donor cfDNA samples (N=72). FIG. 1F, provides a plot of the same defined Spearman correlation coefficient (p) in cancer cfDNA samples with significant copy number changes (defined as the top 10% samples with the greatest copy ratio difference, i.e., log₂(copy ratio)≥2.44, N=31). Grey shades-probability density of ρ across the respective cohort. Dark grey marker-mean±standard error of the mean of ρ across the cohort. The vertical dashed grey lines represent the lower (261 bp) and upper (310 bp) limit of the selected bin Ψ. FIG. 1G, provides a chart summarizing the median of p and P value for each fragment length bin in the healthy (HD) and cancer (C) cohort respectively. In FIGS. 1A-1G, n.s.=P>0.05. *=P≤0.05. **=P≤0.01. ***=P≤0.001.

FIGS. 2A-2I provide plots, box plots, and charts showing TuFEst method validation and comparisons. FIG. 2A, provides a plot showing TuFEst and ichorCNA tumor fraction (TF) estimation in breast cancer cfDNA samples (N=194). The x-axis represents the TF assessed based on a matching WES sample (˜150×): the y-axis represents the estimated-TF using ULP-WGS data. The line represents the diagonal line y=x. FIG. 2B, provides a box plot showing the absolute error of TuFEst (using mean estimator, darker grey and on the right) and ichorCNA (lighter grey and on the left) across eight cancer types. The line indicates the mean absolute error. FIG. 2C, provides a chart summarizing the mean absolute error for TuFEst (expected TF) and ichorCNA across eight cancer types. FIG. 2D, provides a chart summarizing the maximum underestimation error for TuFEst and ichorCNA across the cancer types. FIG. 2E, provides a receiver operating characteristic (ROC) curve representing the accuracy for detecting breast cancer from cfDNA for different TF values (0.5% to 15%) using TuFEst (expected TF), ichorCNA and DELFI. Each group consisted of cfDNA samples from a panel of downsampled healthy donors (˜0.2×, N=360) and in-silico simulated cancers, mixing cfDNA data from cancer and healthy donors to the desired TF (˜0.2×, N=72). The x-axis represents the specificity (1-false positive rate): the y-axis represents the sensitivity (true positive rate). The ROC curve averaged across 10 random split test sets is plotted. FIG. 2F, provides a chart summarizing the classification performance using the area under the ROC curve (AUC) for various tumor fractions (TFs) (average and range over 10 random splits of the healthy donors for training DELFI). FIG. 2G, provides a box plot showing sensitivity (the y-axis) for detecting breast cancer at various TFs in the cfDNA (the x-axis) for TuFEst (expected TF), ichorCNA and DELFI when the false positive rate is set to 1%, using the same data shown in FIG. 2E. FIG. 2H provides a plot showing TuFEst vs. ichorCNA in cfDNA TF estimation (using matching whole-exome sequencing (WES) as the ground truth). The ichorCNA method tended to severely underestimate the tumor fraction for some cfDNA samples, possibly due to the dominant copy neutral loss of heterozygosity, where total copy ratio signals are diluted. On the contrary, when taking into account fragment length signals, TuFEst successfully rescued the tumor fraction (TF) for most cases. FIG. 2I provides a box plot summarizing the underestimation error for cfDNA samples from 8 cancer types using ichorCNA and TuFEst. TuFEst had statistically significant less underestimation than ichorCNA (P=0.00013). In FIGS. 2A-2I, n.s.=P>0.05, *=P≤0.05, **=P≤0.01, ***=P≤0.001.

FIGS. 3A-3E provide box plots, plots, and charts showing the application of TuFEst in studying TF dynamics across multiple samples from the same breast cancer patient. FIG. 3A, provides a box plot showing a sensitivity (the y-axis) for detection of breast cancer across a wide range of TFs (from 5×10⁻⁵ to 10%, x-axis) using TuFEst (expected TF), ichorCNA and DELFI, setting the false positive rate to 1%. The healthy controls (N=25) were derived by downsampling a randomly chosen low pass WGS data (˜4.0×) from a healthy donor to ˜0.2×. Multiple high TF cancers (TF≥65%, N=5) and one healthy donor were used in the in-silico mixing experiments. FIG. 3B, provides a box plot showing cfDNA TFs estimated by TuFEst (y-axis) from a cohort of breast cancer patients receiving different TKI therapies. cfDNA samples are classified into 3 groups based on the timing relative to the therapy: (1) Pre-treatment: prior to receiving any treatments (N=6): (2). On-treatment (effective phase): no clinical signals of relapse (N=30): (3). End- or post-therapy: close to end of therapy (<10 days) or post-therapy. Switch of therapy indicates relapse (N=38). FIG. 3C, provides a plot showing dynamics of tumor fraction (TF) from cfDNA across 7 serial cfDNA samples from a breast cancer patient that received various TKI therapies (ONC154152). The x-axis represents days after diagnosis: the y-axis represents the estimated TF from TuFEst using the ULP-WGS data. Marker and whisker-TuFEst TF expected value and 95% confidence interval. The vertical light-grey line represents the start date of each treatment, and the darker-grey line represents the end date of each treatment. The bottom schematic and chart describe the treatment history. FIG. 3D, provides a plot, schematic, and chart similar to that depicted in the left panel, but for a different breast cancer patient (ONC69469) with 5 serial cfDNA samples. FIG. 3E provides a plot showing serial TF estimates from cfDNA across 13 serial cfDNA samples from a breast cancer patient receiving a CDK4/6 inhibitor (RA_1598). Arrows below the x-axis indicate the dates on which the cfDNA and CT-scan were able to detect cancer relapse, respectively. In FIGS. 3A-3E, n.s.=P>0.05, *=P<0.05. **=P≤0.01. ***=P≤0.001.

FIGS. 4A-4G provide plots and box-plots showing 1-500 bp fragment length distribution across various cancer types. FIGS. 4A-4G (left panels) provide plots showing fragment length distribution of 1-500 bp (normalized against the total number of fragments ≤1000 bp) in high-TF (4th quartile), low-TF (2nd quartile) cancer cfDNA samples and cfDNA samples from healthy donors (N=72). TF of each cancer cfDNA sample was assessed by ABSOLUTE using WES data (˜150×). FIGS. 4A-4G, (left panel insets): distribution of 261-310 bp fragments. FIGS. 4A-4G (right panels), provide box plots showing fraction of 261-310 bp fragments in high-TF, low-TF cancer cfDNA samples, and cfDNA samples from healthy donors (N=72). In FIGS. 4A-4G, n.s.=>0.05. *=P≤0.05. **=P≤0.01. ***=P≤0.001.

FIGS. 5A-5G provide plots showing signal-to-noise ratio across various cancer types. FIGS. 5A-5G provide plots showing signal-to-noise ratios (SNR) of fragments between 50 and 500 bp (binned in 10 bp, x-axis) in cancer cfDNA samples. Shading-probability density of SNR across each respective cancer cohort. Markers-mean of SNR across the cohort. Whiskers-one standard error (standard deviation divided by square root of the cohort size). The vertical dashed grey lines represent the lower (261 bp) and upper (310 bp) limit of the selected bin.

FIG. 6 provides a schematic illustrating the underlying probabilistic model of the TuFEst algorithm.

FIGS. 7A-7G provide plots and box-plots showing comparisons of TF accuracy between TuFEst and ichorCNA across various cancer types. FIGS. 7A-7G (left panels) provide plots showing TuFEst and ichorCNA tumor fraction (TF) estimation in real cancer cfDNA samples. The x-axis represents the TF assessed by matching WES (˜150×): the y-axis represents the estimated-TF using ULP-WGS. The line represents the diagonal line y=x. FIGS. 7A-7G (right panels) provide box plots showing the absolute error of TuFEst (using mean estimator) and ichorCNA, for each cancer type.

FIGS. 8A-8G provide plots and charts showing comparisons of cancer detection power among TuFEst, ichorCNA and DELFI across various cancer types across various tumor fractions (TF). FIGS. 8A-8G provide ROC curves representing the accuracy for detecting various cancer types of various TF in cfDNA (0.5%, 1%, 3%, 5%, 10%, 15%, as shown in the charts) for TuFEst (using mean estimator), ichorCNA and DELFI. Each TF group consisted of cfDNA samples from a panel of downsampled healthy donors (˜0.2×, N=360) and in-silico cancers (˜0.2×, N=72). The in-silico cancers of expected TF were generated through in-silico admixture experiments using multiple high TF cancer cfDNA samples of various cancer types (TF≥30%, N=14, 3, 7, 8, 6, 3, 3 for prostate, bladder, colon, head-and-neck, bile duct, skin, stomach respectively) and a panel of healthy donors (N=72). The x-axis represents the specificity (1-false positive rate): the y-axis represents the sensitivity (true positive rate). The ROC curve averaged across 10 random split test sets is plotted.

FIGS. 9A-9G provide box plots showing comparisons of sensitivity among TuFEst, ichorCNA and DELFI when setting false positive rate to be 1%. FIGS. 9A-9G provide sensitivity box plots (the y-axis) comparing the accuracy for detecting various cancer types of various TF in cfDNA (0.5%, 1%, 3%, 5%, 10%, 15%, the x-axis) for TuFEst (using mean estimator, left), ichorCNA (middle) and DELFI (right). Each TF group consists of cfDNA samples from a panel of downsampled healthy donors (˜0.2×, N=360) and in-silico cancers (˜0.2×, N=72). The in-silico cancers of expected TF were generated through in-silico admixture experiments using multiple high TF cancer cfDNA samples of various cancer types (TF ≥30%, N=14, 3, 7, 8, 6, 3, 3 for prostate, bladder, colon, head-and-neck, bile duct, skin, stomach respectively) and a panel of healthy donors (N=72). In FIGS. 9A-9G. n.s.=P>0.05. *=P<0.05. **=P≤0.01, ***=P≤0.001.

FIG. 10 provides plots showing allelic and total copy ratio of a cfDNA sample from a breast cancer with frequent loss of heterozygosity (LOH). FIG. 10 (left panel) provides the allelic and total copy ratio plot of the same cfDNA sample from the breast cancer patient whose total copy ratio signals were diluted due to LOH, which led to underestimation of tumor fraction (TF). The plot shows major (higher allelic copy ratio: >1) and minor (lower allelic copy ratio; <1) allelic copy ratio across the genome. The x-axis represents the chromosome: the y-axis represents the copy ratio. FIG. 10 (Right panel) provides a histogram showing the cumulative distribution of allelic copy ratio across the genome.

FIGS. 11A-11C provide plots relating to cancer samples with either copy number and/or fragment length abnormalities. FIGS. 11A-11C (top panels), provide plots showing the relative cancer concentration (defined as log₂(copy ratio)) across the genome (binned in 5Mbp) of cfDNA samples from an ovarian cancer (FIG. 11B), a breast cancer (FIG. 11C), a chronic lymphocytic leukemia patient (Richter's transformation: FIG. 11A) (cancer samples correspond to lighter grey points), and healthy donors (N=72, darker grey points) are shown. The mean of log₂(copy ratio) within each genomic segment are plotted as horizontal lines. FIGS. 11A-11C (bottom panels) provide plots showing the proportion of 261-310 bp fragments across the genome (binned in 5Mbp) of cfDNA samples from an ovarian cancer (FIG. 11B), a breast cancer (FIG. 11C), a chronic lymphocytic leukemia patient (Richter's transformation: FIG. 11A) (cancer samples correspond to lighter grey points), and healthy donors (N=72, darker grey points) are shown. The mean proportion of 261-310 bp fragments within each genomic segment are plotted as horizontal lines.

FIG. 12 provides a plot showing fraction of cancers without significant somatic copy number alterations (SCNA) (range of log₂(copy ratio)<0.1) in 33 The Cancer Genome Atlas (TCGA) cancer types. Fraction of cancers without significant SCNA in 33 TCGA cancer types (the x-axis) is shown. Beta-binomial distribution is assumed on the observed fraction for each cancer type. The cancers shown in the plot include glioblastoma multiforme (GBM), ovarian serious cystadenocarcinoma (OV), testicular germ cell tumors (TGCT), skin cutaneous melanoma (SKCM), lung adenocarcinoma (LUAD), breast invasive carcinoma (BRCA), lung squamous cell carcinoma (LUSC), uterine carcinosarcomas (UCS), cervical squamous cell carcinoma and endocervical adenocarcinoma (CESC), head and neck squamous cell carcinoma (HNSC), adenoid cystic carcinoma (ACC), uveal melanoma (UVM), esophageal carcinoma (ESCA), kidney renal clear cell carcinoma (KIRC), bladder urothelial carcinoma (BLCA), stomach adenocarcinoma (STAD), liver hepatocellular carcinoma (LIHC), sarcoma (SARC), rectum adenocarcinoma (READ), brain lower grade glioma (LGG), kidney renal papillary cell carcinoma (KIRP), cholangiocarcinoma (CHOL), colon adenocarcinoma (COAD), mesothelioma (MESO), lymphoid neoplasm diffuse large B-cell lymphoma (DLBC), phenochromocytoma and paraganglioma (PCPG), kidney chromophobe (KICH), prostate adenocarcinoma (PRAD), pancreatic adenocarcinoma (PAAD), uterine corpus endometrial carcinoma (UCEC), acute myeloid leukemia (LAML), thymoma (THYM), and thyroid carcinoma (THCA).

FIGS. 13A-13G provide plots showing that having a pre-cancer sample from a patient significantly improves cancer detection sensitivity in extremely low TF cfDNA samples. FIGS. 13A-13G provide plots showing sensitivity (the y-axis) for detecting breast cancer of extremely TF in cfDNA (5×10⁻⁵, 1.2-4, 2.7e-4, 6.3e-4, 0.15%, 0.34%, 0.79%, 1.8%, 4.3%, 10%, the x-axis) for TuFEst (using mean estimator, left), ichorCNA (middle) and DELFI (right) when the false positive rate is set to 1%, while only using downsampled ULP-WGS data (N=5) from one healthy donor as the healthy cohort. Multiple high TF (TF≥15%, N=5, 5, 5, 5, 5, 5, 4 for prostate, bladder, colon, head-and-neck, bile duct, skin, stomach respectively) cancers and one healthy donor were used in the in-silico admixture experiments.

FIG. 14 illustrates a block diagram of a system, with which some embodiments may operate, for analyzing sequencing data for a plurality of polynucleotides for obtaining tumor fraction (TF).

FIG. 15 provides a flowchart of a process that may be implemented in some embodiments to evaluate tumor fraction (TF) for determining whether the sequencing data came from cancer cells.

FIG. 16 illustrates an exemplary implementation of a computing device that may be used in a system implementing techniques described herein.

### DETAILED DESCRIPTION OF THE INVENTION

The disclosure features compositions and methods that are useful for determining the fraction of tumor-derived DNA (tumor fraction: TF) in cell free DNA (cfDNA). The methods involve calculating the fraction of tumor-derived DNA in the cfDNA using a combination of copy number alteration data and fragment length distribution data.

The invention is based, at least in part, upon the development of a method called TuFEst (Tumor Fraction Estimator), a computational approach for cancer detection and tumor burden estimation from whole genome sequencing (e.g., ultra-low coverage whole genome sequencing, such as to a coverage of about 0.1 or 0.2×) of minimally invasive cell-free DNA. By integrating copy number variation and altered fragment length, TuFEst achieved high detection sensitivity and accurate tumor fraction (TF) estimation across a range of TFs down to 0.1% across various cancer types). As described in the Examples provided herein, TuFEst is a unified physically-informed computational approach for cancer detection and tumor burden estimation through sensitive and accurate estimate of tumor fraction in circulating cell free DNA (cfDNA). TuFEst allowed for detection of cancer and/or tumor burden based upon ultra-low coverage whole genome sequencing (˜0.1×, median: 0.24×: range: 0.055-3.4×) data prepared from cell-free DNA. In embodiments, the TuFEst method is used with sequencing data having about, at least about, or no more than about 0.01, 0.05, 0.1, 0.5, 1, 2, 3, or 5× genome- or exome-wide sequencing coverage. By synergistically integrating copy number variation and altered fragment length data, TuFEst achieved high detection sensitivity and accurate tumor fraction (TF) estimation across a range of TFs down to 0.1% across various cancer types. The method allows for detecting cancer at early stages or upon recurrence, which is critical to decrease cancer morbidity and mortality.

Advantageously, circulating cell-free DNA (cfDNA) provides a noninvasive route for cancer detection and burden estimation since tumor-derived DNA (ctDNA) can be differentiated from normal DNA based on specific genetic alteration (mutations, copy number variation, altered methylation patterns, altered fragment length or nucleosome occupancy). Moreover, the use of ULP-WGS by TuFEst is more cost-effective for broad application than other methods including methylation-based assays or deep coverage sequencing by targeted panels.

### A Tumor Fraction Estimator (TuFEst)

Tumor fraction (TF) estimation may be leveraged for early cancer diagnosis and early detection of resistant clones that may develop under treatment. Available methods can estimate TF based on features of sequencing data from cfDNA and ctDNA. However, methods estimating TF exclusively based on SCNAs can lose tumor signal in either copy number-quiet tumors or tumors dominated by copy-neutral loss-of-heterozygosity, and methods estimating TF exclusively based on fragment length may exclude potentially valuable information if fragment lengths are not chosen that correspond to a high signal-to-noise ratio (SNR) between cancerous and non-cancerous gene expression samples. Thus, there is a need to develop a tumor fraction estimator that can use information from both fragment length distributions and somatic copy number alterations as input to improve accuracy and/or sensitivity of prediction while avoiding potential drawbacks encountered when either is used alone.

The Examples provided herein demonstrate the advantages of leveraging both SCNA and altered fragment length, rather than using either feature by itself, and computationally combining them in a way that provides a synergistic effect through orthogonal constraints that complement each other and together achieve a higher sensitivity for detecting cancer. In particular, among other things, it was found that the methods provided herein can improve the sensitivity and accuracy of cancer detection, through metrics such as SNR and correlation coefficients based on SCNAs and fragment length. Further, in embodiments, the methods are cost-effective and non-invasive and can detect cancer recurrence earlier than standard clinical tests. The methods provided herein may be leveraged for detecting and/or measuring disease progression for any number of cancer types, such as, for example, prostate: colon: bladder: skin: bile duct: stomach; and head-and-neck.

In various aspects, the disclosure provides TuFEst: a Bayesian model (e.g., an interpretable Bayesian graphical model) that integrates both SCNA and fragment length for cancer detection through accurate tumor fraction (TF) estimation in cfDNA. The model combines genetic and nongenetic signatures in a physically-informed way. In particular TuFEst integrates the evidence and uncertainties from both SCNA and fragment length distributions and produces a joint posterior distribution over the TF values and the predicted total copy-number profile, from which is then extracted the marginal posterior distribution over the TF values. In some instances, only fragment length is used for accurate tumor fraction (TF) estimation.

Cell free DNA (cfDNA) contains genetic-level alterations (e.g., somatic copy number alterations (SCNAs), gene fusions, mutations, loss of heterozygosity, aneuploidy, deletions, insertions, inversions, translocations, amplifications, etc.) and nongenetic alterations (e.g., methylation signals or fragment-length distribution signals), as well as epigenetic-level signatures. Since this epigenetic-level signature information is known to indicate cell-of-origin, DNA released from cancer cells is expected to be different from that released from healthy blood cells. For example, Cell free DNA (cfDNA) fragments have “footprints” of nucleosome positions that inform the cell-of-origin for the cfDNA. Therefore, leveraging genetic and nongenetic signatures, as in the TuFEst method provided herein, allows for more sensitive cancer detection using cfDNA than that possible using either signature-type alone. In particular, TuFEst allows for detection of the fraction of DNA in a cfDNA sample that is derived from a tumor cell(s) (i.e., tumor fraction).

TuFEst addresses two important limitations of methods that rely only on the detection of somatic copy number alterations (SCNAs). First, such methods cannot detect copy number-quiet tumors. Through analyzing 9613 TCGA SNP-array data, it was found that on average about 7.2% of cancers are copy number-quiet or dominated by copy-neutral loss-of-heterozygosity, with some cancer types having extremely high fractions of copy number-quiet tumors (e.g., 68% in thyroid carcinoma). Finally, SCNA background noise limits its power in distinguishing clonal from sub-clonal copy-number events, which complicates its ability for TF estimation and thus the detection limit of TF is ˜3%.

The methods of the disclosure involve characterizing somatic copy number alterations and/or fragment length distribution present in a polynucleotide sample (e.g., a cell free DNA sample) and then using this information to determine the tumor fraction of the polynucleotide sample. In embodiments, the methods can detect a tumor fraction of about, of at least about, and/or of less than about 1e-5, 5e-5, 1e-4, 1.2e-4, 2.7e-4, 6.3e-4, 1e-3, 1.5e-3, 3.4e-3, 5e-3, 7.9e-3, 1e-2, 1.8e-2, 2e-2, 3e-2, 4e-2, 4.3e-2, 5e-1, 6e-2, 7e-2, 8e-2, 9e-2, 1e-1, 2e-1, 3e-1, 4e-1, 5e-1, 6e-1, 7e-1, 8e-1, 9e-1, or 1.

In embodiments, characterizing the length distribution present in the polynucleotide sample involves determining the number of DNA fragments in a polynucleotide sample falling within a range of sizes (i.e., a fragment-size bin). In embodiments, the fragment-size bin or collection of fragment-size bins is selected such that the fragments are associated with a high signal-to-noise ratio (SNR) and/or a high correlation coefficient with somatic copy number alterations (i.e., “cancer concentration”) and/or with tumor fraction in a polynucleotide sample. In embodiments, cancer concentration is log2(copy ratio). In embodiments, the bins collectively or individually cover DNA fragments with sizes of, or a size span of about, at least about, or no more than about 5 bp, 10 bp, 15 bp, 20 bp, 25 bp, 30 bp, 35 bp, 40 bp, 45 bp, 50 bp, 75 bp, 100 bp, 150 bp, 200 bp, 300 bp, 400 bp, 500 bp, or 1000 bp. In various instances, the range of sizes is from about 261 bp to about 310 bp, or from about 281 bp to about 290 bp. In some cases, the range of sizes is from about or at least about 50 bp, 100 bp, 150 bp, 200 bp, 210 bp, 220 bp, 230 bp, 240 bp, 250 bp, 260 bp, 270 bp, 280 bp, 290 bp, 300 bp, 310 bp, 320 bp, 330 bp, 340 bp, 350 bp, 400 bp, or 450 bp to about or at least about 100 bp, 150 bp, 200 bp, 210 bp, 220 bp, 230 bp, 240 bp, 250 bp, 260 bp, 270 bp, 280 bp, 290 bp, 300 bp, 310 bp, 320 bp, 330 bp, 340 bp, 350 bp, 400 bp, 450 bp, 500 bp, or 550 bp.

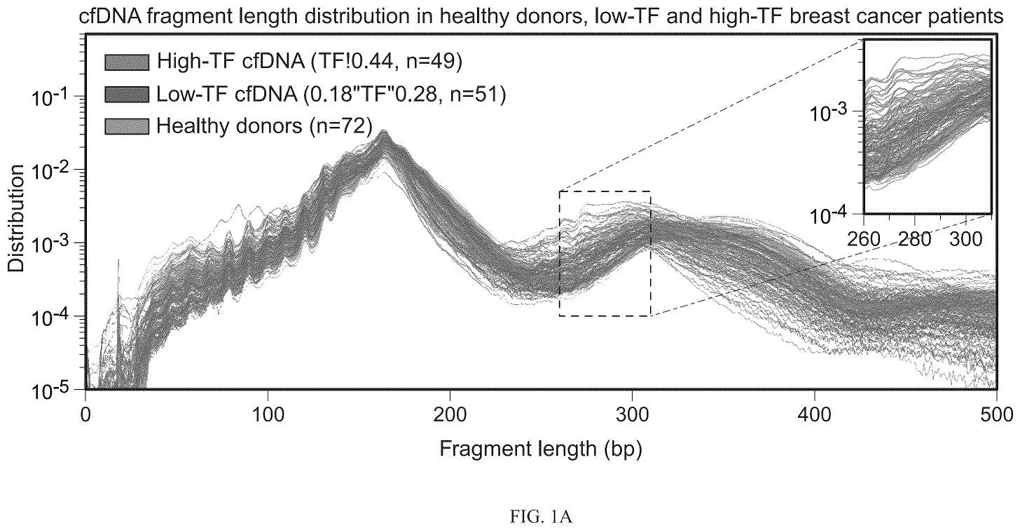

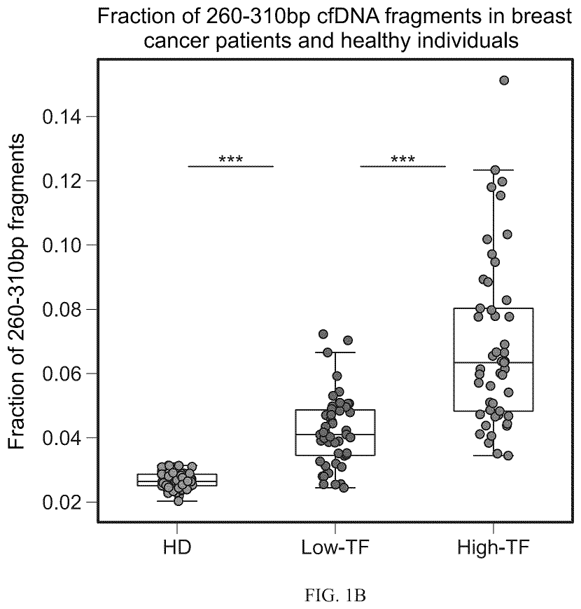

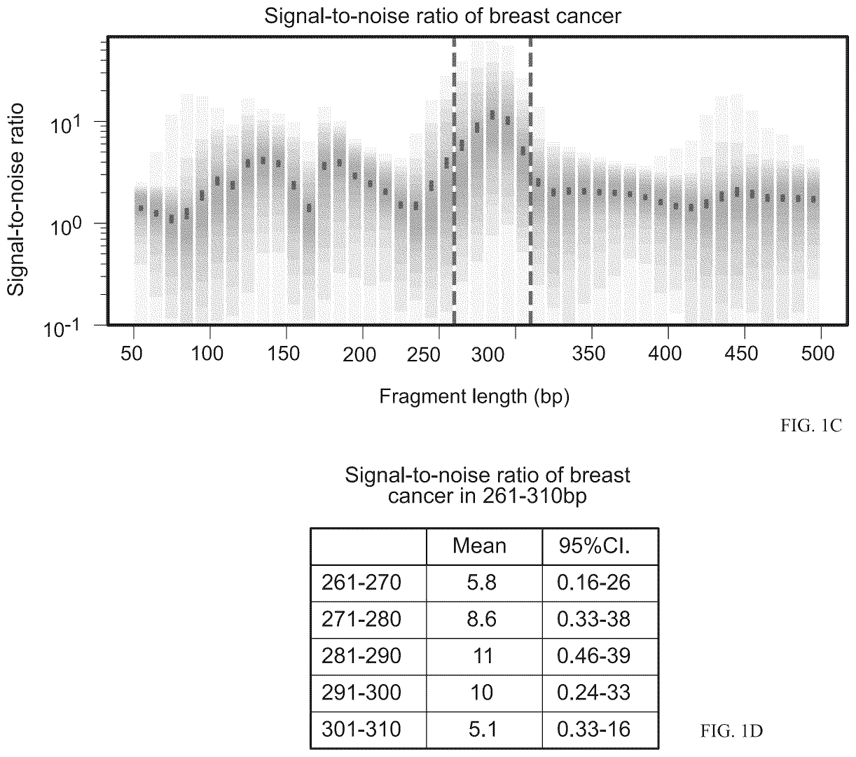

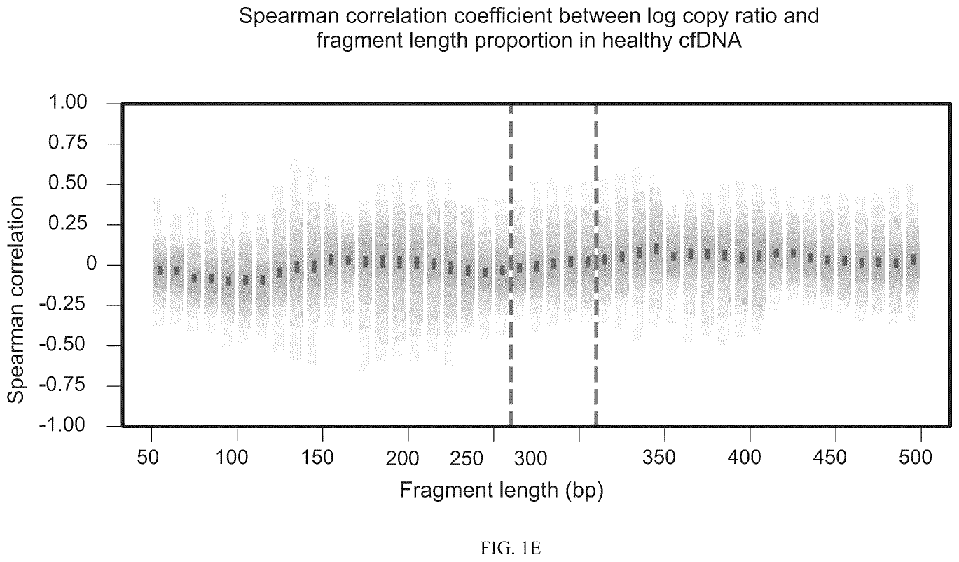

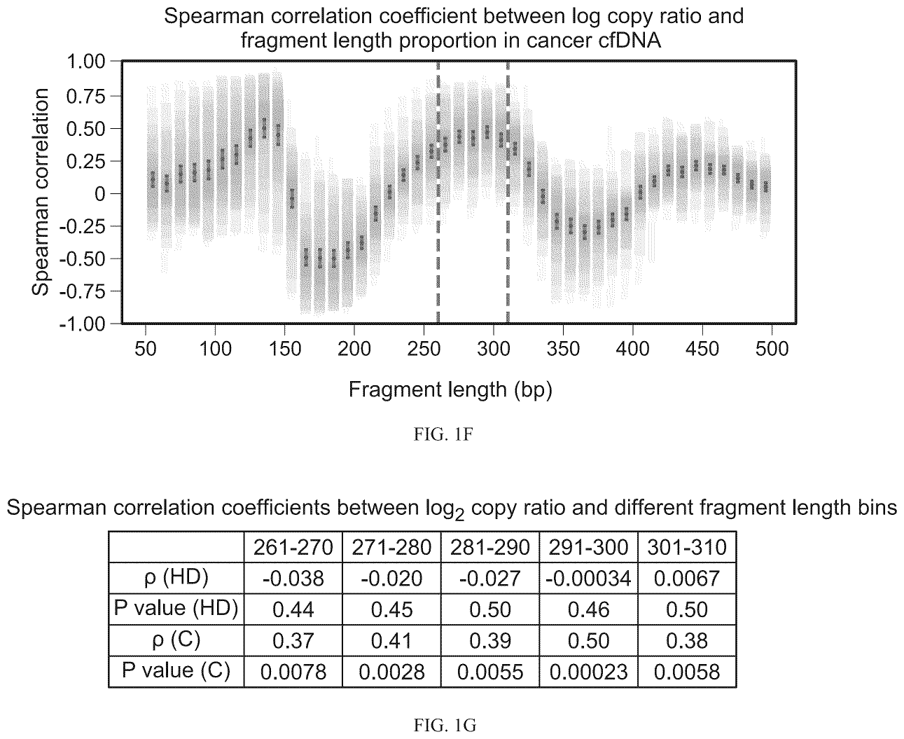

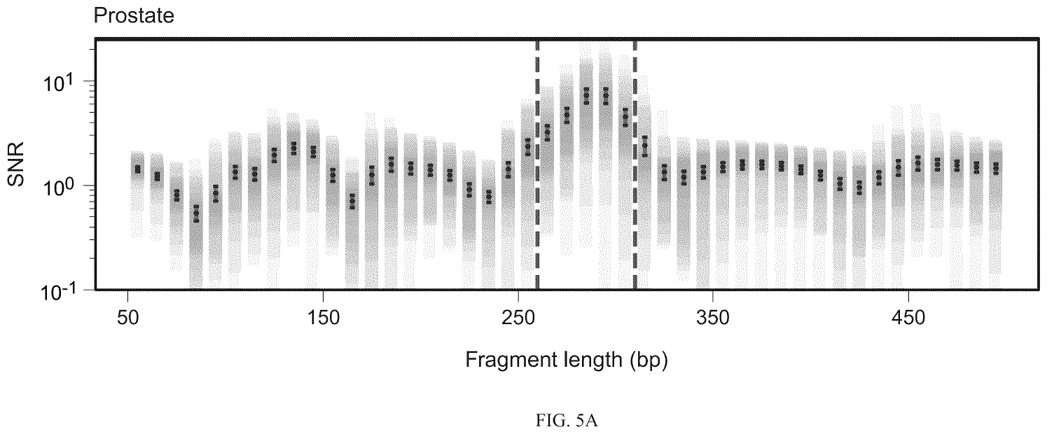

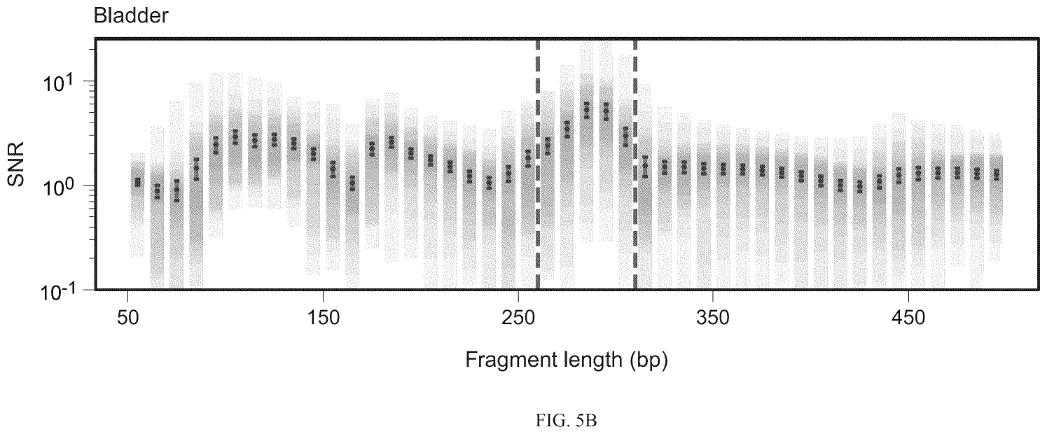

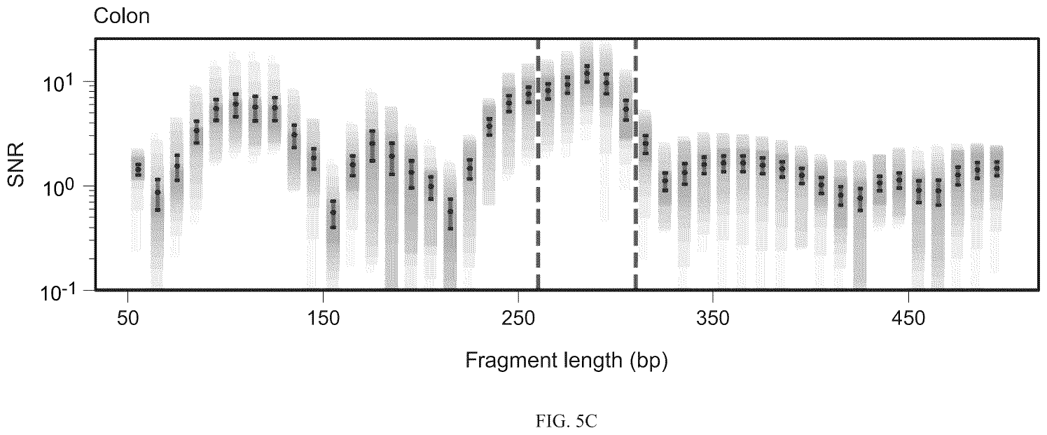

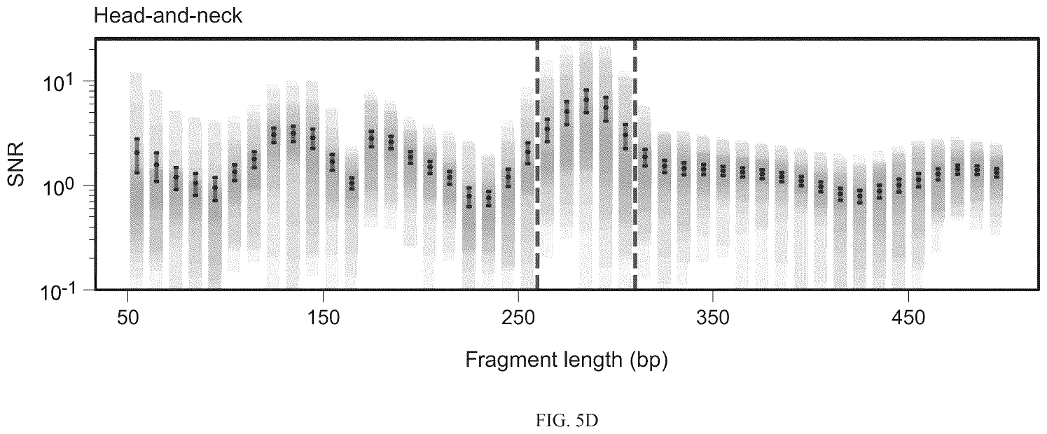

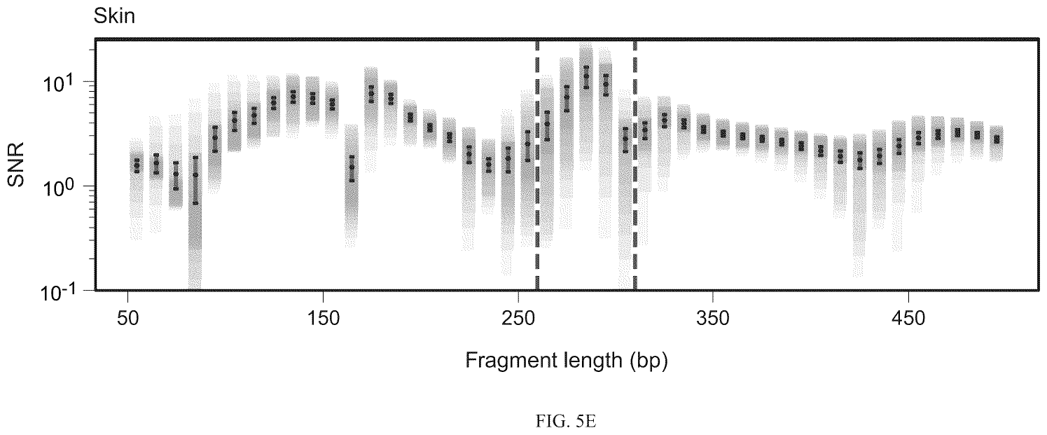

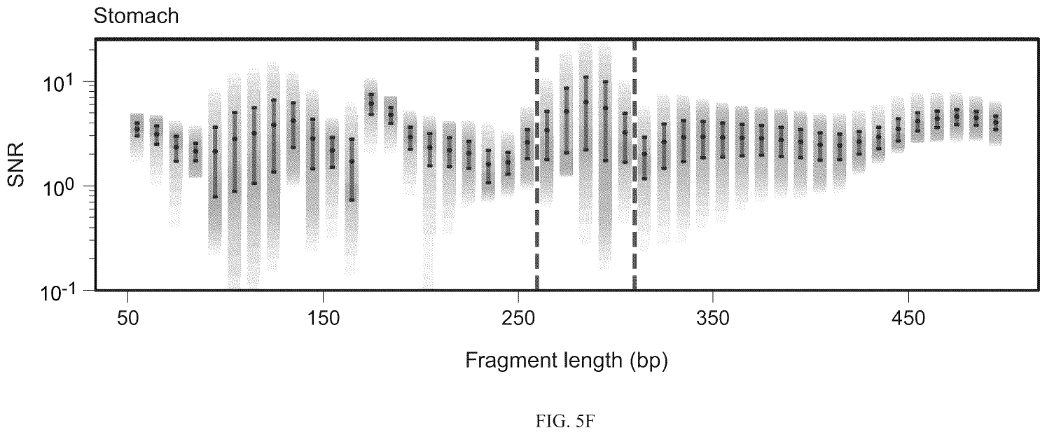

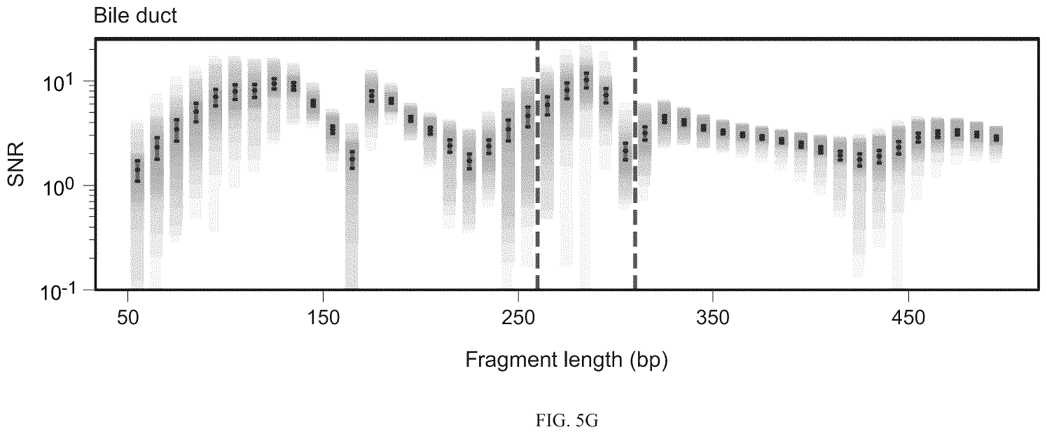

In embodiments, the selected bins are contiguous, non-contiguous, or a combination thereof. In embodiments, the bin(s) is selected to provide a higher average signal-to-noise ratio than alterative bin selections. In embodiments, the alternative bins are those adjacent to a contiguous set of bins having the higher average signal-to-noise ratio, such that the selected bin(s) corresponds to a local maximum signal-to-noise radio (SNR) for adjacent bins (see, e.g., FIGS. 1B, and 5A-5G). The SNR is a significance metric and, in embodiments, is calculated for bins that are about, at least about, or no more than about 5 bp, 10 bp, 15 bp, 20 bp, 25 bp, 30 bp, 35 bp, 40 bp, 45 bp, 50 bp, 75 bp, 100 bp, 150 bp, 200 bp, 300 bp, 400 bp, 500 bp, or 1000 bp in size. SNR_{i,j} is the fraction of those fragments j in sample i minus the average fraction in a panel of healthy donors, and then divided by the standard deviation of the fraction in the healthy cohort. A higher SNR for a fragment length bin(s) indicates that that fragment length bin(s) corresponds to increased tumor fraction. In embodiments, the SNR is about or at least about 1, 2, 3, 4, 5, 6, 7, 8, 9, 10, 11, 12, 13, 14, or 15. In embodiments, the bins are selected such that at least one of the bins has a SNR of about or at least about 1, 2, 3, 4, 5, 6, 7, 8, 9, 10, 11, 12, 13, 14, or 15 and all other bins, optionally where the other bins are contiguous with the one bin, have an SNR of about or at least about 1, 2, 3, 4, 5, 6, 7, 8, 9, 10, 11, 12, 13, 14, or 15.

In embodiments, the correlation coefficient is a Spearman correlation coefficient. In some instances, the Spearman correlation coefficient is calculated between log₂(copy ratio) and fragment length distribution. In embodiments, for a given cancer sample t and fragment length r, the Spearman correlation coefficient between a log_2-transformed copy ratio (log₂(copy ratio)) and the fraction of fragments with length r across the genomic segments with the most extreme copy number alterations (top 10% for amplifications or bottom 10% for deletions) is calculated. In embodiments, the value and/or absolute value of the correlation coefficient (e.g., a Spearman correlation coefficient) is about or at least about 0.1, 0.15, 0.2, 0.25, 0.3, 0.35, 0.4, 0.45, or 0.5.

In various instances, the characterizing involves sequencing the polynucleotide sample using any of the methods provided herein to a coverage of about, at least about, and/or no more than about 1e-8, 1e-7, 1e-6, 1e-5, 1e-4, 1e-3, 1e-2, 0.05×, 0.1×, 0.2×, 0.3×, 0.4×, 0.5×, 1×, 2×, 3×, 4×, 5×, 6×, 7×, 8×, 9×, 10×, 20×, 30×, 40×, 50×, 60×, 70×, 80×, 90×, 100×, or more. In embodiments, the methods involve isolating polynucleotides (e.g., DNA (e.g., cfDNA) or RNA) from a biological sample (e.g., a blood sample), sequencing the polynucleotides, analyzing the sequence data using models described herein, and determining the tumor fraction present in the polynucleotide sample.

In various cases, the absolute error with which a tumor fraction is determined is about, at least about, or no more than about 0%, 1%, 2%, 3%, 4%, 5%, 6%, 7%, 8%, 9%, 10%, 11%, 12%, 13%, 14%, 15%, 20%, 25%, or 30%.

In embodiments, the method involves comparing sequence data to a reference normal sample. In some cases, the reference normal sample is a polynucleotide sample (e.g., a cfDNA sample) from a healthy subject or a subject prior to having a neoplasm.

In another embodiment, the disclosure provides a method of diagnosing cancer, as described further below, in a subject by detecting the tumor fraction of a polynucleotide sample from a subject. In yet another embodiment, the disclosure provides a method, as described further below, of determining the efficacy of a treatment and/or an agent for treatment of a cancer by characterizing tumor fraction in a polynucleotide sample from the subject.

### Implementation of TuFEst Algorithm

Techniques operating according to the principles described herein may be implemented in any suitable manner. Included in the discussion above are a series of flow charts showing the steps and acts of various processes for analyzing sequencing data to better estimate tumor fraction (TF) and increase the sensitivity of cancer detection and cancer progression. The processing and decision blocks of the flow charts above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that the flow charts included herein do not depict the syntax or operation of any particular circuit or of any particular programming language or type of programming language. Rather, the flow charts illustrate the functional information one skilled in the art may use to fabricate circuits or to implement computer software algorithms to perform the processing of a particular apparatus carrying out the types of techniques described herein. It should also be appreciated that, unless otherwise indicated herein, the particular sequence of steps and/or acts described in each flow chart is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more processors, causes the one or more processors to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way: all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionalities may be implemented in a single functional facility. It should also be appreciated that, in some implementations, some of the functional facilities described herein may be implemented together with or separately from others (i.e., as a single unit or separate units), or some of these functional facilities may not be implemented.

### Computer Systems

The present disclosure also relates to a computer system involved in carrying out the methods of the disclosure relating to both computations and sequencing.

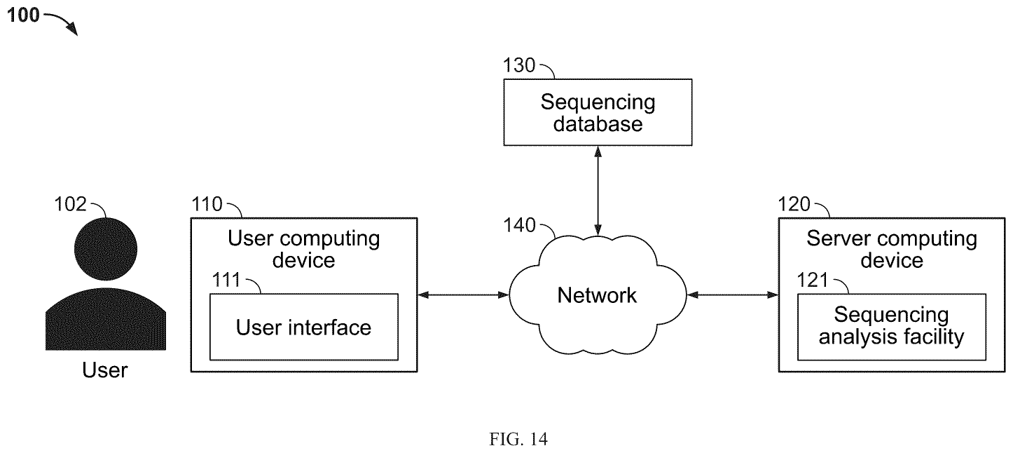

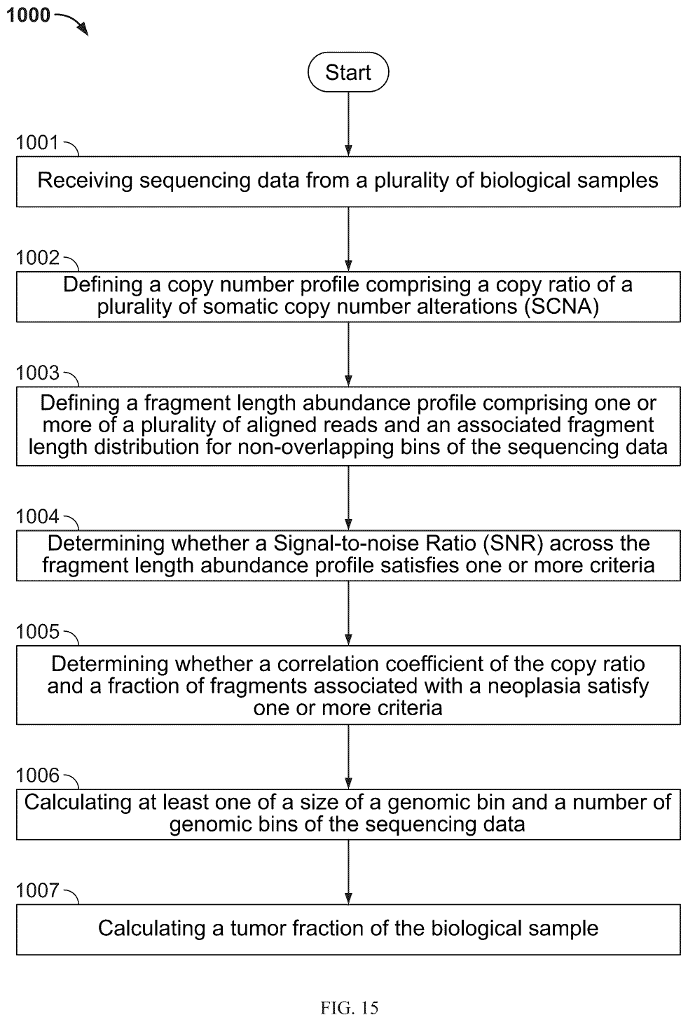

FIG. 14 illustrates a block diagram of a system 100 with which some embodiments may operate. The system 100 can analyze sequencing data for a plurality of polynucleotides for obtaining tumor fraction (TF). The system 100 can include a user computing device 110, which may be a desktop or laptop personal computer, smart mobile phone, server, or other suitable device. The user computing device 110 may include a user interface 111 by which the user 102 may interact with the user computing device 110. For example, the user 102 can use the user interface 111 to interface with the sequencing database 130 or sequencing analysis facility 121 of the server computing device 120, or to control any of the TuFEst algorithm parameters. For example, the user 102 may operate the user interface 111 to initiate analysis of a polynucleotide from the sequencing database 130 and display analysis results such as, for example, Signal-to-Noise Ratio (SNR), false positive (FP) rate, or Spearman Correlation Coefficients of the somatic copy number alterations (SCNA) and/or fragment length distribution data in the user interface 111. The user 102 may further operate the user interface 111 to receive data, such as the analysis results, from the sequencing analysis facility 121. The user 102 may additionally or alternatively operate the user interface 111 to calculate TF obtained from the sequencing database 130, such as output to the user 102 in another interface. Those values may be provided to the sequencing analysis facility 121. As a further example, the user 102 may operate the user interface 111 to initiate analysis of the polynucleotides by the sequencing database 130 and provision of analysis results (e.g., TF from SCNA and/or fragment length distribution) from the sequencing database 130 to the sequencing analysis facility 121. Results of analysis of the results (received from the sequencing database 130 or from the user interface 111) by the sequencing analysis facility 121 may be output to the user interface 111, such as by being received at the user interface 111 and displayed on the device 110. In some embodiments, as mentioned above, the user interface 111 may include a web interface, such as one or more web pages into which values may be output and which may display results of the analysis by the sequencing analysis facility 121, but embodiments are not so limited. The user interface 111 may accept input in a variety of different formats, such as through speech recognition, text input, or other means, as embodiments are not limited in this respect.

The system 100 can include a server computing device 120, which may include a sequencing analysis facility 121 configured to analyze factors (e.g., derived from the polynucleotides, such as by the sequencing database 130) for the user 102 to determine information regarding TF, such as to quantify TF. In some embodiments, the sequencing analysis facility 121 may receive information on the factors from the sequencing database 130 and/or from the user interface 111. In some embodiments, the sequencing analysis facility 121 may output TF characteristics such as SNR, false positive (FP) rate, or Spearman Correlation Coefficient that satisfy predetermined criteria for the TF.

The system 100 can include a network 140 to facilitate communications among the sequencing database 130, the user computing device 110, and the server computing device 120. The network 140 can be or include any one or more wired and/or wireless, local- and/or wide-area network, including one or more enterprise networks and/or the Internet.

While the example of FIG. 14 includes the client interface on a device 110 separate from the sample analyzer 112, it should be appreciated that embodiments are not so limited. In other embodiments, the user interface 111 may be an interface of the sequencing database 130 and may be operated by the user 102. Additionally or alternatively, while the sequencing analysis facility 121 is illustrated on a different computing device from the user computing device 110 and the sequencing database 130, embodiments are not so limited. In other embodiments, the sequencing analysis facility 121 may be implemented on the client computing device or the sequencing database 130. In some embodiments, the user interface 111 may not be separate from the sequencing analysis facility 121, but instead may be implemented as a single program or software application. In some embodiments, a sequencing database 130 may include the user interface 111 and the sequencing analysis facility 121, and the user interface 111 and sequencing analysis facility 121 may be implemented within the same program or application executed on the sequencing database 130.

FIG. 15 provides a flowchart of a process 1000 that may be implemented in some embodiments to evaluate tumor fraction (TF) for determining whether the sequencing data came from cancer cells. Process 1000 can be implemented in some embodiments by the sequencing analysis facility 121 of the server computing device 120, which can output selected copy number profiles and fragment length abundance profiles that satisfy predetermined criteria for TF. In some embodiments described herein, information regarding selected copy number profiles and fragment length abundance profiles and their expression in normal and cancerous cells is analyzed in specific ways and characterized to estimate TF. In step 1001, sequencing data is received from a plurality of biological samples, in particular ULP-WGS data from cfDNA and/or ctDNA. Ultra-low coverage (˜0.1×, median: 0.24×: range: 0.055-3.4×) whole genome sequencing data (ULP-WGS) can be more cost-effective than use of other deep coverage sequencing data.

In some embodiments, preliminary analysis may be performed by the sequencing analysis facility 121 in steps 1002 and 1003, wherein a copy number profile and a fragment length abundance profile (e.g., via a user interface, via a network communication, or otherwise), may be defined, wherein the copy number profile may comprise a copy ratio of a plurality of somatic copy number alterations (SCNA), and the fragment length abundance profile may comprise one or more of a plurality of aligned reads and an associated fragment length distribution for non-overlapping bins of the sequencing data. These profiles are among those utilized for calculating SNR and a correlation coefficient in steps 1004 and 1005, respectively, then determining whether they satisfy one or more criteria. In particular, the experiments outlined in the Examples provided herein investigate fragment bins (261-310 bp) in a fraction of cancers without significant SCNA (range of log2(copy ratio)<0.1) in 33 TCGA cancer types to determine criteria for estimating TF.

In some embodiments, as provided in steps 1006 and 1007, at least one of a size of a genomic bin and a number of genomic bins of the sequencing data are obtained from the fragment length distribution and SCNA of the sequencing data, then used to calculate a TF for each of the plurality of biological samples, which may be calculated by the sequencing analysis facility 121 for each measured profile. This calculation can also be performed for any number of other parameters, such as the SNR and correlation coefficients. In some embodiments, the TF is generated automatically by sequencing analysis facility 121 (e.g., via an algorithm) or manually generated by a user (e.g., via user interface 111). A computer system (or digital device), such as an exemplary computer system in FIG. 14, may be used to receive, transmit, display and/or store results, analyze the results, and/or produce a report of the results and analysis. A computer system may be understood as a logical apparatus that can read instructions from media (e.g., software) and/or network port (e.g., from the internet), which can optionally be connected to a server having fixed media. A computer system may comprise one or more of a CPU, disk drives, input devices such as keyboard and/or mouse, and a display (e.g., a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present disclosure can be transmitted over such networks or connections (or any other suitable means for transmitting information, including but not limited to mailing a physical report, such as a print-out) for reception and/or for review by a receiver. The receiver can be but is not limited to an individual, or electronic system (e.g., one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules, and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the disclosure. A client-server architecture is a network architecture in which each computer or processor on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the disclosure, the server computer handles all of the database functionality. The client computer can have software that handles all the front-end data management and can also receive data input from users.

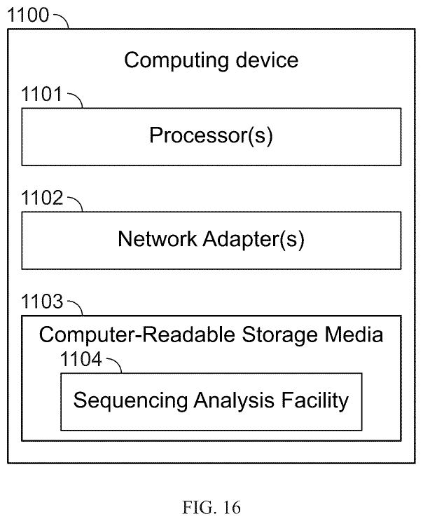

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as computer-readable storage media 1103 of FIG. 16 described below (i.e., as a portion of a computing device 1100) or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, including the exemplary computer system of FIG. 14, or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, a computer-readable storage medium accessible via one or more networks and accessible by the device/processor, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing devices (co-located or geographically distributed) dedicated to executing the techniques described herein, one or more Field-Programmable Gate Arrays (FPGAs) for carrying out the techniques described herein, or any other suitable system.

FIG. 16 illustrates one exemplary implementation of a computing device in the form of a computing device 1100 that may be used in a system implementing techniques described herein, although others are possible. It should be appreciated that FIG. 16 is intended neither to be a depiction of necessary components for a computing device to execute a sequencing analysis facility 1104 in accordance with the principles described herein, nor a comprehensive depiction.

Computing device 1100 may comprise at least one processor 1101, a network adapter 1102, and computer-readable storage media 1103. Computing device 1100 may be, for example, a desktop or laptop personal computer, a personal digital assistant (PDA), a smart mobile phone, a server, a wireless access point or other networking element, or any other suitable computing device. Network adapter 1102 may be any suitable hardware and/or software to enable the computing device 1100 to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable media 1103 may be adapted to store data to be processed and/or instructions to be executed by processor 1101. Processor 1101 enables processing of data and execution of instructions. The data and instructions may be stored on the computer-readable storage media 1103.

The data and instructions stored on computer-readable storage media 1103 may comprise computer-executable instructions implementing techniques which operate according to the principles described herein. In the example of FIG. 16, computer-readable storage media 1103 stores computer-executable instructions implementing various facilities and storing various information as described above. Computer-readable storage media 1103 may store sequencing analysis facility 1104, which may implement one or more of the techniques described herein.

While not illustrated in FIG. 16, a computing device may additionally have one or more components and peripherals, including input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computing device may receive input information through speech recognition or in other audible format.

A machine readable medium which may comprise computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables: copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations. A computer can transform data into various formats for display. A graphical presentation of the results of a calculation can be displayed on a monitor, display, or other visualizable medium (e.g., a printout). In some embodiments, data or the results of a calculation may be presented in an auditory form.

### Types of Samples

This disclosure provides methods to extract and sequence a polynucleotide present in a sample. In one embodiment, the samples are biological samples generally derived from a human subject, preferably as a bodily fluid (such as ascites, blood, plasma, pleural fluid, serum, cerebrospinal fluid, phlegm, saliva, stool, urine, semen, prostate fluid, breast milk, or tears, or tissue sample (e.g., a tissue sample obtained by biopsy). In a further embodiment, the samples are biological samples derived from an animal, preferably as a bodily fluid (such as blood, cerebrospinal fluid, phlegm, saliva, or urine) or tissue sample (e.g., a tissue sample obtained by biopsy). In still another embodiment, the samples are biological samples from in vitro sources (such as cell culture medium). Cell free (cfDNA) attached to a substrate may be first suspended in a liquid medium, such as a buffer or a water, and then subject to sequencing and/or analysis. In yet another embodiment, the sample contains DNA within a cell, which may be extracted, sequenced and subject to the same analysis. In some instances, the sample is a biopsy (e.g., a needle biopsy) or a tissue section.

### Reference Sequences

In certain aspects, the instant disclosure provides methods and kits that involve and/or allow for assessment of the presence or absence of one or more sequence variants (e.g., somatic copy number alterations) and/or mutations in a test subject, tissue, cell, or sample, as compared to a corresponding reference sequence. In particular embodiments, a subject, tissue, cell and/or sample is assessed for one or more variants and/or sites of copy number variation within the sequences/sequence locations (e.g., motif A as defined below). The reference sequence can correspond to cell free DNA from a healthy subject and/or from a subject prior to having and/or being diagnosed with a neoplasm. A reference sequence can correspond to cell free DNA from a patient-matched normal control.

### Sequencing

In various aspects, the methods provided herein involve sequencing of a sample. In some embodiments, the sequencing is whole-genome sequencing (WGS) or whole-exome sequencing (WES). The sequencing is performed upon a test sample for the purpose of detecting fragment length distributions and somatic copy number alterations in a sample (e.g., in cell free DNA). In certain embodiments, the sequencing can be performed with or without amplification of a sample to be sequenced. In embodiments, a sample is sequenced to a coverage of about, at least about, and/or no more than about 0.01×, 0.05×, 0.1×, 0.2×, 0.3×, 0.4×, 0.5×, 1×, 2×, 3×, 4×, 5×, 6×, 7×, 8×, 9×, 10×, 20×, 30×, 40×, 50×, 60×, 70×, 80×, 90×, 100×, or more.

Whole genome sequencing (also known as “WGS”, full genome sequencing, complete genome sequencing, or entire genome sequencing) is a process that involves sequencing a complete DNA sequence of an organism's genome. A common strategy used for WGS is shotgun sequencing, in which DNA is broken up randomly into numerous small segments, which are sequenced. Sequence data obtained from one sequencing reaction is termed a “read.” The reads can be assembled together based on sequence overlap. The genome sequence is obtained by assembling the reads into a reconstructed sequence.

Whole exome sequencing (“WES”) is a technique used to sequence all the expressed genes in a cell or subject (known as the exome). It includes first selecting only that portion of a polynucleotide sample that encodes proteins (e.g., cDNA, or a subset of a cfDNA sample), and then sequencing using any DNA sequencing technology well known in the art or as described herein. In a human being, there are about 180,000 exons, which constitute about 1% of the human genome, or approximately 30 million base pairs. In some embodiments, to sequence the exons of a genome, fragments of double-stranded genomic DNA are obtained (e.g., by methods such as sonication, nuclease digestion, or any other appropriate methods). Linkers or adapters are then attached to the DNA fragments, which are then hybridized to a library of polynucleotides designed to capture only the exons. The hybridized DNA fragments are then selectively isolated and subjected to sequencing using any sequencing method known in the art or described herein.

Sequencing may be performed on any high-throughput platform. Methods of sequencing oligonucleotides and nucleic acids are well known in the art (see, e.g., WO93/23564, WO98/28440 and WO98/13523: U.S. Pat. Nos. 5,525,464:5,202,231:5,695,940:4,971,903; 5,902,723:5,795,782:5,547,839 and 5,403,708: Sanger et al., Proc. Natl. Acad. Sci. USA 74:5463 (1977): Drmanac et al., Genomics 4:114 (1989): Koster et al., Nature Biotechnology 14:1123 (1996): Hyman, Anal. Biochem. 174:423 (1988): Rosenthal, International Patent Application Publication 761107 (1989); Metzker et al., Nucl. Acids Res. 22:4259 (1994): Jones, Biotechniques 22:938 (1997): Ronaghi et al., Anal. Biochem. 242:84 (1996): Ronaghi et al., Science 281:363 (1998): Nyren et al., Anal. Biochem. 151:504 (1985): Canard and Arzumanov, Gene 11:1 (1994): Dyatkina and Arzumanov, Nucleic Acids Symp Ser 18:117 (1987): Johnson et al., Anal. Biochem. 136:192 (1984); and Elgen and Rigler, Proc. Natl. Acad. Sci. USA 91 (13): 5740 (1994), all of which are expressly incorporated herein by reference). In one embodiment, the sequencing of a DNA fragment is carried out using commercially available sequencing technology SBS (sequencing by synthesis) by Illumina. In another embodiment, the sequencing of the DNA fragment is carried out using chain termination method of DNA sequencing. In yet another embodiment, the sequencing of the DNA fragment is carried out using one of the commercially available next-generation sequencing technologies, including SMRT (single-molecule real-time) sequencing from Pacific Biosciences, Ion Torrent™ sequencing from ThermoFisher Scientific, Pyrosequencing (454) from Roche, and SOLID® technology from Applied Biosystems. Any appropriate sequencing technology may be chosen for sequencing.

For purpose of this disclosure, the term “amplification” means any method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. A preferred amplification method is PCR. Typically, the amplification of a sample results in an exponential increase in copy number of the amplified sequences. Amplification may involve thermocycling or isothermal amplification (such as through the methods RPA or LAMP).

Design and use of oligonucleotides for amplification and/or sequencing is within the knowledge of one of ordinary skill in the art. Oligonucleotides can be modified by any of a number of art-recognized moieties and/or exogenous sequences, e.g., to enhance the processes of amplification, sequencing reactions, and/or detection. Exemplary oligonucleotide modifications that are expressly contemplated for use with the oligonucleotides of the instant disclosure include, e.g., fluorescent and/or radioactive label modifications: labeling one or more oligonucleotides with a universal amplification sequence (optionally of exogenous origin) and/or labeling one or more oligonucleotides of the instant disclosure with a unique identification sequence (e.g., a “bar-code” sequence, optionally of exogenous origin), as well as other modifications known in the art and suitable for use with oligonucleotides.

Patient and/or Treatment Monitoring

In various aspects, the disclosure provides methods for monitoring a patient for a neoplasia and/or monitoring the efficacy of a neoplasia (e.g., a cancer or tumor) treatment and/or resistance to therapy in a subject being treated for a neoplasia. The methods involve measuring tumor fraction in cell free DNA collected from the subject according to the methods provided herein. In some instances, the methods provided herein are used to monitor tumor fraction in polynucleotides (e.g., cfDNA) in a liquid biopsy of a patient as part of routine monitoring (e.g., as part of a routine physical) for a neoplasia.

The methods described herein include methods for the treatment of a neoplasia (e.g., a cancer or tumor). Generally, the methods include administering a therapeutically effective amount of a treatment as described herein, to a subject who is in need of, or who has been determined to be in need of, such treatment. The methods further involve measuring a tumor fraction in polynucleotide samples (e.g., cell free DNA in a blood sample) from the subject according to the methods provided herein.

The methods provided herein can be used for clinical cancer management, such as for the diagnosis of a cancer, for detection of a cancer, for minimal residual disease monitoring, for tracking of treatment efficacy, or for detecting a cancer in a subject. Tumor fraction (TF) of cell free DNA is used in various embodiments as a biomarker to diagnose cancer, detect cancer relapse, or detect treatment failure. In embodiments, cell free DNA TF dynamics are monitored to track and/or measure tumor burden and/or indicate treatment efficacy. Cell free DNA TF dynamics aligns well with tumor burden, and is, therefore, a biomarker to indicate cancer relapse due to drug resistance. In various instances, the methods provided herein are used for early screening and/or in clinical cancer management.

In various instances, the methods provided herein are used to measure tumor fraction in a polynucleotide sample taken from a subject. The measurements can be taken periodically at regular intervals. In some cases, measurements are taken about, at least about, or no more than about 1, 2, 3, 4, 5, 6, 7, 8, 9, 10, 11, 12, 13, 14, or 15 times every or about every 1 day, 3 days, 1 week, 1 month, 2 months, 3 months, 4 months, 5 months, 6 months, 7 months, 8 months, 9 months, 10 months, 11 months, 12 months, 1.5 years, 2 years, 3 years, 4 years, or 5 years. In some instances, measurements are taken as part of a routine physical. In some cases, tumor fraction is measured as part of a process to monitor a subject for cancer. The polynucleotide sample in various cases is cfDNA.

The methods of the disclosure advantageously allow for monitoring the efficacy of a neoplasia treatment. In some cases, a treatment is characterized as ineffective (i.e., a tumor is resistant to treatment or has developed resistance to treatment) if tumor fraction increases in a subject being administered the treatment. In embodiments, if a treatment is characterized as ineffective in a subject (i.e., the tumor is resistant to treatment or has developed resistance to treatment), the treatment is changed to an alternative treatment. The increase or decrease in various instances is statistically significant. In some instances, a treatment is characterized as effective if the tumor fraction in cell free DNA is maintained beneath a threshold and is characterized as ineffective if tumor fraction is not maintained beneath the threshold. In various instances, the threshold is about, at least about, or no more than about 5%, 10%, 15%, 20%, 25%, 30%, 35%, 40%, 45%, or 50%. In some cases, a treatment is characterized as ineffective if the tumor fraction increases significantly. In some instances, a treatment is characterized as ineffective if an increase in tumor fraction of about, or at least about 1%, 2%, 3%, 4%, 5%, 10%, 20%, 30%, 40%, 50%, 1×, 2×, 3×, 4×, 5×, 10×, 100×, or more is measured.

The methods of the disclosure can include diagnosing a subject as having a neoplasia if cell free DNA collected from the subject is found to contain a statistically significant non-zero fraction of tumor DNA.

In some instances, the ability of the methods provided herein to detect low tumor fraction levels can be improved by sequencing a polynucleotide sample (e.g., a cfDNA sample) from a matched normal sample and using the matched normal sample in the methods provided herein as a reference sample. The matched normal sample can be a sample from a subject prior to having a neoplasia.

Treatments amenable to monitoring using the methods of the disclosure include, but are not limited to, chemotherapy, radiotherapy, immunotherapy, surgery, or various other methods available to a skilled practitioner or described herein.

### Cancer Treatments

Methods of inhibiting and/or treating cancer and tumors in individuals with cancer or a predisposition for developing cancer as identified by methods of the disclosure are also contemplated.

In embodiments, the subject has been diagnosed with a neoplasm (e.g., a cancer) or is at risk of developing a neoplasm (e.g., a cancer or tumor). The subject, in various instances, is a human, dog, cat, horse, or any animal. Illustrative neoplasms include breast cancer, esophageal cancer, head-and-neck cancer, pancreatic cancer, skin cancer, colorectal cancer, hepatocellular cancer, bladder cancer, bile duct cancer, luminal and non-luminal bladder cancer, basal bladder cancer, muscle-invasive bladder cancer, and non-muscle-invasive bladder cancer, pancreatic cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, liver cancer, cervical cancer, uterine cancer, testicular cancer, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). In embodiments, the neoplasia may be colon adenocarcinoma (COAD), stomach adenocarcinoma (STAD), stomach cancer, and uterine corpus endometrial carcinoma (UCEC). In embodiments, the neoplasia may be a liquid tumor such as, for example, leukemia or lymphoma. In embodiments, the cancer is a bile duct, bladder, breast, colon, head-and-neck, liver and/or intrahepatic bile ducts cancer, ovarian, skin, or stomach cancer, or a chronic lymphocytic leukemia (Richter's transformation).

The therapeutic agent is for example, a chemotherapeutic agent, radiation, or immunotherapy. Any suitable therapeutic treatment for a particular cancer may be administered. Examples of chemotherapeutic agents include, but are not limited to, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol™), pilocarpine, prochloroperazine, rituximab, tamoxifen, taxol, topotecan hydrochloride, trastuzumab, vinblastine, vincristine and vinorelbine tartrate.

For therapeutic use, administration often begins at the detection or surgical removal of tumors. This is followed by boosting doses until at least symptoms are substantially abated and for a period thereafter.

The pharmaceutical compositions for therapeutic treatment are intended for parenteral, topical, nasal, oral or local administration. Preferably, the pharmaceutical compositions are administered parenterally, e.g., intravenously, subcutaneously, intradermally, or intramuscularly. The compositions may be administered at the site of surgical excision to induce a local immune response to the tumor. The disclosure provides compositions for parenteral administration that comprise a solution of the peptides and vaccine compositions are dissolved or suspended in an acceptable carrier, preferably an aqueous carrier. A variety of aqueous carriers may be used, e.g., water, buffered water, 0.9% saline, 0.3% glycine, hyaluronic acid, and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, toxicity adjusting agents, wetting agents, and the like, for example, sodium acetate, sodium lactate, sodium chloride, potassium chloride, calcium chloride, sorbitan monolaurate, triethanolamine oleate, etc.

In an advantageous embodiment, the cancer therapeutic is an immunotherapeutic (e.g., an antibody, such as pembrolizumab). The immunotherapeutic may be a cytokine therapeutic (such as an interferon or an interleukin), a dendritic cell therapeutic or an antibody therapeutic, such as a monoclonal antibody. In a particularly advantageous embodiment, the immunotherapeutic is a neoantigen (see, e.g., U.S. Pat. No. 9,115,402 and US Patent Publication Nos. 2011/0293637, 2016/0008447, 2016/0101170, 2016/0331822 and 2016/0339090).

In particular embodiments, treatments for adrenal, breast, cervical, colon, endometrial, rectal or stomach cancer are contemplated.

For adrenal cancer, surgery is recommended to remove the entire adrenal gland. Standard treatment options for adrenocortical carcinoma (ACC) include, but are not limited to, chemotherapy with mitotane, chemotherapy with mitotane plus streptozotocin or mitotane plus etoposide, doxorubicin, and cisplatin, radiation therapy to bone metastases and/or surgical removal of localized metastases, particularly those that are functioning.

For breast cancer, local therapies such as surgery and radiation are recommended. Breast cancer may also be treated systemically by chemotherapy, hormone therapy (such as, but not limited to, tamoxifen, toremifene, fulvestrant or aromatase inhibitors) or targeted therapy (such as, but not limited to, monoclonal antibodies or other therapeutics that target a HER2 protein, a m Tor protein or cyclin-dependent kinases, or kinase inhibitors). If the breast cancer is a BRCA cancer, the cancer may be treated and/or prevented by a mastectomy, sapingo-oophorectomy, or hormonal therapy medicines, such as selective estrogen receptor modulators or aromatase inhibitors. Hormonal therapy medicines include, but are not limited to, tamoxifen, raloxifene, exemestane or anastrozole.

Cervical cancer may be treated by surgery, radiation, chemotherapy, or targeted therapy (such as an angiogenesis inhibitor). Cervical squamous cell carcinoma may be treated by cryosurgery, laser surgery, loop electrosurgical excision procedure (LEEP/LEETZ), cold knife conization or a simple hysterectomy (as the first treatment or if the cancer returns after other treatments). Endocervical adenocarcinoma (CESC) may be treated by surgery or radiation.

Colon cancer may be treated by surgery or chemotherapy. Some common regimens for treating colon cancer include, but are not limited to: OLFOX: leucovorin, 5-FU, and oxaliplatin (Eloxatin): FOLFIRI: leucovorin, 5-FU, and irinotecan (Camptosar): CapeOX: capecitabine (Xeloda) and oxaliplatin: FOLFOXIRI: leucovorin, 5-FU, oxaliplatin, and irinotecan: One of the above combinations plus either a drug that targets VEGF (bevacizumab [Avastin], ziv-aflibercept [Zaltrap], or ramucirumab [Cyramza]), or a drug that targets EGFR (cetuximab [Erbitux] or panitumumab [Vectibix]): 5-FU and leucovorin, with or without a targeted drug; Capecitabine, with or without a targeted drug: Irinotecan, with or without a targeted drug; Cetuximab alone: Panitumumab alone: Regorafenib (Stivarga) alone; and/or Trifluridine and tipiracil (Lonsurf).

Endometrial cancer may be treated by surgery, chemotherapy, and radiation. Uterine corpus endometrial carcinoma (UCEC) is the most common type of endometrial cancer. Operative procedures used for managing endometrial cancer include the following: exploratory laparotomy, total abdominal hysterectomy, bilateral salpingo-oophorectomy, peritoneal cytology, and pelvic and para-aortic lymphadenectomy. Chemotherapeutic medications such as cisplatin can be used in the management of endometrial carcinoma. Standard treatment options for uterine carcinosarcoma (UCS) include surgery (total abdominal hysterectomy, bilateral salpingo-oophorectomy, and pelvic and periaortic selective lymphadenectomy), surgery plus pelvic radiation therapy, surgery plus adjuvant chemotherapy or surgery plus adjuvant radiation therapy (EORTC-55874).

Rectal cancer may be treated by surgery, chemotherapy, and radiation. Some common regimens for treating rectal cancer include, but are not limited to: FOLFOX: leucovorin, 5-FU, and oxaliplatin (Eloxatin): FOLFIRI: leucovorin, 5-FU, and irinotecan (Camptosar): CapeOX; capecitabine (Xeloda) and oxaliplatin: FOLFOXIRI: leucovorin, 5-FU, oxaliplatin, and irinotecan; One of the above combinations, plus either a drug that targets VEGF (bevacizumab [Avastin], ziv-aflibercept [Zaltrap], or ramucirumab [Cyramza]), or a drug that targets EGFR (cetuximab [Erbitux] or panitumumab [Vectibix]): 5-FU and leucovorin, with or without a targeted drug: Capecitabine, with or without a targeted drug: Irinotecan, with or without a targeted drug: Cetuximab alone: Panitumumab alone: Regorafenib (Stivarga) alone; and/or Trifluridine and tipiracil (Lonsurf).

Stomach cancer may be treated by surgery, radiation, chemotherapy, or targeted therapy (such as a monoclonal antibody or other therapeutics that target a HER2 protein or a VEGF receptor). Drugs approved for stomach cancer include, but are not limited to, Capecitabine (Xeloda). Cisplatin (Platinol), Cyramza (Ramucirumab), Docetaxel, Doxorubicin Hydrochloride, 5-FU (Fluorouracil Injection), Fluorouracil Injection, Herceptin (Trastuzumab), Irinotecan Hydrochloride, Leucovorin Calcium, Mitomycin C, Mitozytrex (Mitomycin C), Mutamycin (Mitomycin C), Ramucirumab, Taxotere (Docetaxel) and Trastuzumab and may be administered individually or in a combination thereof.

The therapeutics of the present disclosure may be delivered in a particle and/or nanoparticle delivery system. Several types of particle and nanoparticle delivery systems and/or formulations are known to be useful in a diverse spectrum of biomedical applications; and particle and nanoparticle delivery systems in the practice of the instant disclosure can be as in WO 2014/093622 (PCT/US13/74667), herein incorporated by reference.

### Pharmaceutical Compositions

Agents of the present disclosure can be incorporated into a variety of formulations for therapeutic use (e.g., by administration) or in the manufacture of a medicament (e.g., for treating or preventing a neoplasm) by combining the agents with appropriate pharmaceutically acceptable carriers or diluents, and may be formulated into preparations in solid, semi-solid, liquid, or gaseous forms. Examples of such formulations include, without limitation, tablets, capsules, powders, granules, ointments, solutions, suppositories, injections, inhalants, gels, microspheres, and aerosols.

For example, neoplasias described herein may be treated with therapeutic agents such as, for example, immunotherapeutic agents that act by effectively stimulating the immune response, e.g., PD-1/PD-L1 inhibitors (e.g., Pembrolizumab), CDK4/6 inhibitors, and tyrosine kinase inhibitors (TKIs).

In addition to immunotherapeutic treatments, the disclosure includes treatment with additional agents, either alone or in combination with the immunotherapeutic treatment (such as the anti-PD-1/PDL-1 therapeutic agent). Examples of such agents include chemotherapeutic agents including chemotherapeutic alkylating agents such as Cyclophosphamide, Mechlorethamine, Chlorambucil, Melphalan, Monofunctional alkylators, Dacarbazine, nitrosoureas, and Temozolomide (Oral dacarbazine): anthracyclines such as Daunorubicin, Doxorubicin, Epirubicin, Idarubicin, Mitoxantrone, Valrubicin, cytoskeletal disruptor agents (taxanes) such as Paclitaxel, Docetaxel, Abraxane and Taxotere: Epothilones: Histone deacetylase inhibitors such as Vorinostat and Romidepsin: topoisomerase I inhibitors such as Irinotecan and Topotecan: topoisomerase II inhibitors such as Etoposide, Teniposide, and Tafluposide: Kinase inhibitors such as Bortezomib, Erlotinib, Gefitinib, Imatinib, Vemurafenib, and Vismodegib: nucleotide analogs and precursor analog agents such as Azacitidine, Azathioprine, Capecitabine, Cytarabine, Doxifluridine, Fluorouracil, Gemcitabine, Hydroxyurea, Mercaptopurine, Methotrexate, and Tioguanine (formerly Thioguanine): peptide antibiotics such as Bleomycin and Actinomycin: Platinum-based agents such as Carboplatin, Cisplatin, Oxaliplatin: Retinoids such as Retinoids, Tretinoin, Alitretinoin, Bexarotene: Vinca alkaloids and derivatives such as Vinblastine, Vincristine, Vindesine and Vinorelbine: as well as other chemotherapeutic agents including all-trans retinoic acid, Docetaxel, Doxifluridine, Epothilone, Fluorouracil, Methotrexate, and Pemetrexed.

Chemotherapeutic agents for use with the disclosure include any chemical compound used in the treatment of a neoplasia. Chemotherapeutic agents include, but are not limited to, RAF inhibitors (e.g., BRAF inhibitors), MEK inhibitors, PI3K inhibitors and AKT inhibitors. Other chemotherapeutic agents include, without being limited to, the following classes of agents: nitrogen mustards, e.g., cyclophosphamide, trofosfamide, ifosfamide and chlorambucil: nitroso ureas, e.g., carmustine (BCNU), lomustine (CCNU), semustine (methyl CCNU) and nimustine (ACNU): ethylene imines and methyl-melamines, e.g., thiotepa: folic acid analogs, e.g., methotrexate: pyrimidine analogs, e.g., 5-fluorouracil and cytarabine: purine analogs, e.g., mercaptopurine and azathioprine: vinca alkaloids, e.g., vinblastine, vincristine and vindesine; epipodophyllotoxins, e.g., etoposide and teniposide: antibiotics, e.g., dactinomycin, daunorubicin, doxorubicin, epirubicin, bleomycin a2, mitomycin c and mitoxantrone: estrogens, e.g., diethyl stilbestrol: gonadotropin-releasing hormone analogs, e.g., leuprolide, buserelin and goserelin: antiestrogens, e.g., tamoxifen and aminoglutethimide: androgens, e.g., testolactone and drostanolonproprionate: platinates, e.g., cisplatin and carboplatin; and interferons, including interferon-alpha, beta and gamma.

Chemotherapeutic agents include, for example, RAF inhibitors (e.g., Vemurafenib or Dabrafenib), MEK inhibitors, PI3K inhibitors, or AKT inhibitors. The RAF inhibitor is, for example, a BRAF inhibitor. The chemotherapeutic agents can be administered alone or in combination (e.g., RAF inhibitors with MEK inhibitors).

In addition, these modulatory agents can also be administered in combination therapy with, e.g., chemotherapeutic agents, hormones, antiangiogens, radiolabeled, compounds, or with surgery, cryotherapy, and/or radiotherapy. The preceding treatment methods can be administered in conjunction with other forms of conventional therapy (e.g., standard-of-care treatments for cancer well known to the skilled artisan), either consecutively with, pre- or post-conventional therapy.

The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular cancer, being treated, the combined use of immunotherapeutic agent, the extent of the disease and other factors familiar to the physician of skill in the art and can be determined by the physician.

Pharmaceutical compositions can include, depending on the formulation desired, pharmaceutically-acceptable, non-toxic carriers of diluents, which are vehicles commonly used to formulate pharmaceutical compositions for animal or human administration. The diluent is selected so as not to affect the biological activity of the combination. Examples of such diluents include, without limitation, distilled water, buffered water, physiological saline, PBS, Ringer's solution, dextrose solution, and Hank's solution. A pharmaceutical composition or formulation of the present disclosure can further include other carriers, adjuvants, or non-toxic, nontherapeutic, nonimmunogenic stabilizers, excipients, and the like. The compositions can also include additional substances to approximate physiological conditions, such as pH adjusting and buffering agents, toxicity adjusting agents, wetting agents, and detergents.

Further examples of formulations that are suitable for various types of administration can be found in Remington's Pharmaceutical Sciences, Mace Publishing Company, Philadelphia, PA, 17th ed. (1985). For a brief review of methods for drug delivery, see, Langer, Science 249:1527-1533 (1990).

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink.

Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Formulations may be optimized for retention and stabilization in a subject and/or tissue of a subject, e.g., to prevent rapid clearance of a formulation by the subject. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of the agent in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, e.g., having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, case of encapsulation, a half-life in the physiological environment.

Biodegradable polymeric compositions that may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the individual instant disclosure. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

### Pharmaceutical Dosages

Pharmaceutical compositions of the present disclosure containing an agent described herein may be used (e.g., administered to an individual, such as a human individual, in need of treatment) in accord with known methods, such as oral administration, intravenous administration as a bolus or by continuous infusion over a period of time, by intramuscular, intraperitoneal, intracerobrospinal, intracranial, intraspinal, subcutaneous, intraarticular, intrasynovial, intrathecal, topical, or inhalation routes.

Dosages and desired drug concentration of pharmaceutical compositions of the present disclosure may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles described in Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

For in vivo administration of any of the agents of the present disclosure, normal dosage amounts may vary from about 10 ng/kg up to about 100 mg/kg of an individual's and/or subject's body weight or more per day, depending upon the route of administration. In some embodiments, the dose amount is about 1 mg/kg/day to 10 mg/kg/day. For repeated administrations over several days or longer, depending on the severity of the disease, disorder, or condition to be treated, the treatment is sustained until a desired suppression of symptoms is achieved.

An effective amount of an agent of the instant disclosure may vary, e.g., from about 0.001 mg/kg to about 1000 mg/kg or more in one or more dose administrations for one or several days (depending on the mode of administration). In certain embodiments, the effective amount per dose varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg.

An exemplary dosing regimen may include administering an initial dose of an agent of the disclosure of about 200 μg/kg, followed by a weekly maintenance dose of about 100 μg/kg every other week. Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 μg/kg to about 2 mg/kg (such as about 3 μg/kg, about 10 μg/kg, about 30 μg/kg, about 100 μg/kg, about 300 μg/kg, about 1 mg/kg, or about 2 mg/kg) may be used. In certain embodiments, dosing frequency is three times per day, twice per day, once per day, once every other day, once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is easily monitored by conventional techniques and assays. The dosing regimen, including the agent(s) administered, can vary over time independently of the dose used.

Pharmaceutical compositions described herein can be prepared by any method known in the art of pharmacology. In general, such preparatory methods include the steps of bringing the agent or compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

The exact amount of an agent required to achieve an effective amount will vary from subject to subject, depending, for example, on species, age, and general condition of a subject, severity of the side effects or disorder, identity of the particular agent, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of an agent described herein.

A drug of the instant disclosure may be administered via a number of routes of administration, including but not limited to: subcutaneous, intravenous, intrathecal, intramuscular, intranasal, oral, transepidermal, parenteral, by inhalation, or intracerebroventricular.

The FDA-approved drug or other therapy is administered to the subject in an amount sufficient to achieve a desired effect at a desired site (e.g., reduction of cancer size, cancer cell abundance, symptoms, etc.) determined by a skilled clinician to be effective. In some embodiments of the disclosure, the agent is administered at least once a year. In other embodiments of the disclosure, the agent is administered at least once a day. In other embodiments of the disclosure, the agent is administered at least once a week. In some embodiments of the disclosure, the agent is administered at least once a month.

Additional exemplary doses for administration of an agent of the disclosure to a subject include, but are not limited to, the following: 1-20 mg/kg/day, 2-15 mg/kg/day, 5-12 mg/kg/day, 10 mg/kg/day, 1-500 mg/kg/day, 2-250 mg/kg/day, 5-150 mg/kg/day, 20-125 mg/kg/day, 50-120 mg/kg/day; 100 mg/kg/day, at least 10 μg/kg/day, at least 100 μg/kg/day, at least 250 μg/kg/day, at least 500 μg/kg/day, at least 1 mg/kg/day, at least 2 mg/kg/day, at least 5 mg/kg/day, at least 10 mg/kg/day, at least 20 mg/kg/day, at least 50 mg/kg/day, at least 75 mg/kg/day, at least 100 mg/kg/day, at least 200 mg/kg/day, at least 500 mg/kg/day, at least 1 g/kg/day, and a therapeutically effective dose that is less than 500 mg/kg/day, less than 200 mg/kg/day, less than 100 mg/kg/day, less than 50 mg/kg/day, less than 20 mg/kg/day, less than 10 mg/kg/day, less than 5 mg/kg/day, less than 2 mg/kg/day, less than 1 mg/kg/day, less than 500 μg/kg/day, and less than 500 μg/kg/day.

In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of an agent (e.g., a tyrosine kinase inhibitor (TKI), a CDK4/6 inhibitor, etc.) described herein. In certain embodiments, a dose described herein includes independently between 1 mg and 3 mg, inclusive, of an agent (e.g., a tyrosine kinase inhibitor (TKI), a CDK4/6 inhibitor, etc.) described herein. In certain embodiments, a dose described herein includes independently between 3 mg and 10 mg, inclusive, of an agent (e.g., a tyrosine kinase inhibitor (TKI), a CDK4/6 inhibitor, etc.) described herein. In certain embodiments, a dose described herein includes independently between 10 mg and 30 mg, inclusive, of an agent (e.g., a tyrosine kinase inhibitor (TKI), a CDK4/6 inhibitor, etc.) described herein. In certain embodiments, a dose described herein includes independently between 30 mg and 100 mg, inclusive, of an agent (e.g., a tyrosine kinase inhibitor (TKI), a CDK4/6 inhibitor, etc.) described herein.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

It will be also appreciated that an agent (e.g., a tyrosine kinase inhibitor (TKI), a CDK4/6 inhibitor, etc.) or composition, as described herein, can be administered in combination with one or more additional pharmaceutical agents (e.g., therapeutically and/or prophylactically active agents), which are different from the agent or composition and may be useful as, e.g., combination therapies. The agents or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk of developing a disease in a subject in need thereof, in inhibiting the replication of a virus, in killing a virus, etc. in a subject or cell. In certain embodiments, a pharmaceutical composition described herein including an agent (e.g., a tyrosine kinase inhibitor (TKI), a CDK4/6 inhibitor, etc.) described herein and an additional pharmaceutical agent shows a synergistic effect that is absent in a pharmaceutical composition including one of the agent and the additional pharmaceutical agent, but not both.

In some embodiments of the disclosure, a therapeutic agent distinct from a first therapeutic agent of the disclosure is administered prior to, in combination with, at the same time, or after administration of the agent of the disclosure. In some embodiments, the second therapeutic agent is selected from the group consisting of a chemotherapeutic, an antioxidant, an anti-inflammatory agent, an antimicrobial, a steroid, etc.

The agent or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease described herein. Each additional pharmaceutical agent may be administered at a dose and/or on a time schedule determined for that pharmaceutical agent. The additional pharmaceutical agents may also be administered together with each other and/or with the agent or composition described herein in a single dose or administered separately in different doses. The particular combination to employ in a regimen will take into account compatibility of the agent described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, chemotherapeutic agents, other epigenetic modifier inhibitors, etc., other anti-cancer agents, immunomodulatory agents, anti-proliferative agents, cytotoxic agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine kinase inhibitors), modulators of protein stability (e.g., proteasome inhibitors), Hsp90 inhibitors, glucocorticoids, all-trans retinoic acids, and other agents that promote differentiation. In certain embodiments, the agents described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy.

Dosages for a particular agent of the instant disclosure may be determined empirically in individuals who have been given one or more administrations of the agent.

Administration of an agent of the present disclosure can be continuous or intermittent, depending, for example, on the recipient's physiological condition, whether the purpose of the administration is therapeutic or prophylactic, and other factors known to skilled practitioners. The administration of an agent may be essentially continuous over a preselected period of time or may be in a series of spaced doses.

Guidance regarding particular dosages and methods of delivery is provided in the literature: see, for example, U.S. Pat. Nos. 4,657,760; 5,206,344; or 5,225,212. It is within the scope of the instant disclosure that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

### Kits

The instant disclosure also provides kits containing agents of this disclosure for use in the methods of the present disclosure. Kits of the instant disclosure may include one or more containers comprising an agent (e.g., a chemotherapeutic agent) of this disclosure and/or may contain agents (e.g., oligonucleotide primers, probes, etc.) for determining the fraction of cell free DNA in a sample that is derived from a tumor. In some embodiments, the kits further include instructions for use in accordance with the methods of this disclosure. In some embodiments, these instructions comprise a description of administration of the agent to treat or diagnose (e.g., a neoplasia) according to any of the methods of this disclosure. In some embodiments, the instructions comprise a description of how to calculate tumor fraction in cfDNA, for example in an individual, in a tissue sample, or in a cell, and, in some cases, the instructions may describe how such calculations should inform the treatment of a patient.

The instructions generally include information as to dosage, dosing schedule, and route of administration for the intended treatment. The containers may be unit doses, bulk packages (e.g., multi-dose packages) or sub-unit doses. Instructions supplied in the kits of the instant disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, e.g., a neoplasia, in a subject. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (e.g., the container may be an intravenous solution bag or a vial having a stopper pierceable by a hypodermic injection needle). In certain embodiments, the kit includes at least one active agent (e.g., a chemotherapeutic agent).

Kits may optionally provide additional components such as buffers and interpretive information. Normally, the kit comprises a container and a label or package insert(s) on or associated with the container.

The practice of the present invention employs, unless otherwise indicated, conventional techniques of molecular biology (including recombinant techniques), microbiology, cell biology, biochemistry, and immunology, which are well within the purview of the skilled artisan. Such techniques are explained fully in the literature, such as, “Molecular Cloning: A Laboratory Manual”, second edition (Sambrook, 1989): “Oligonucleotide Synthesis” (Gait, 1984): “Animal Cell Culture” (Freshney, 1987): “Methods in Enzymology” “Handbook of Experimental Immunology” (Weir, 1996): “Gene Transfer Vectors for Mammalian Cells” (Miller and Calos, 1987): “Current Protocols in Molecular Biology” (Ausubel, 1987): “PCR: The Polymerase Chain Reaction”, (Mullis, 1994): “Current Protocols in Immunology” (Coligan, 1991). These techniques are applicable to the production of the polynucleotides and polypeptides of the disclosure, and, as such, may be considered in making and practicing the disclosure. Particularly useful techniques for particular embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their invention.

### EXAMPLES

### Example 1: Tumor Fragment Size Correlated with Tumor Fraction and Relative Copy Number Profile in Cell Free DNA

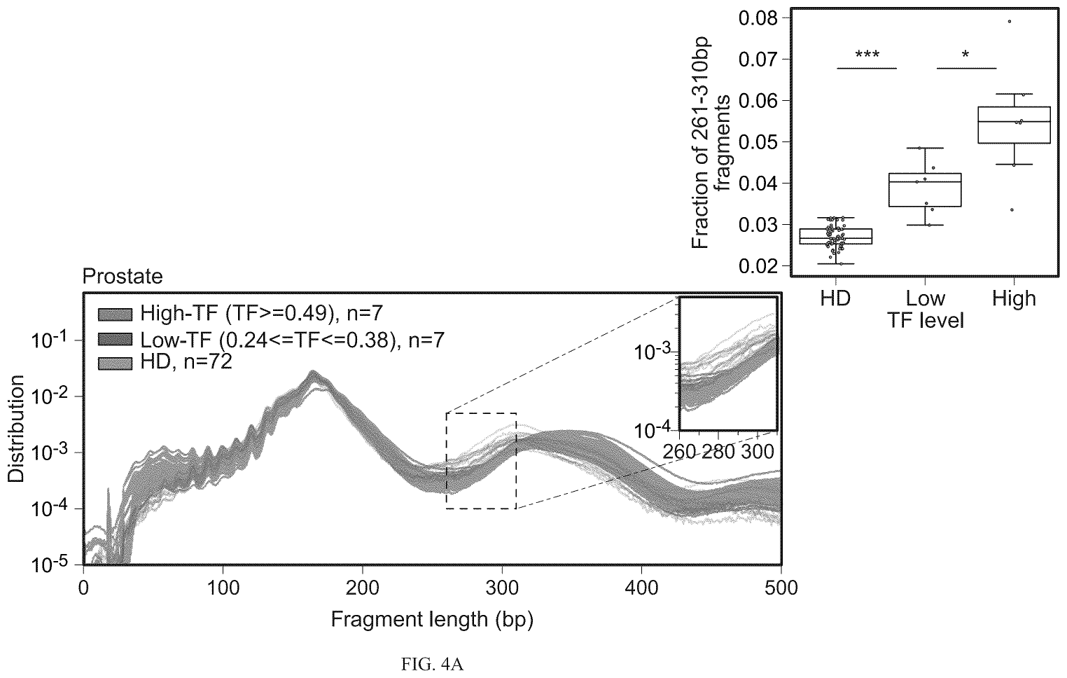

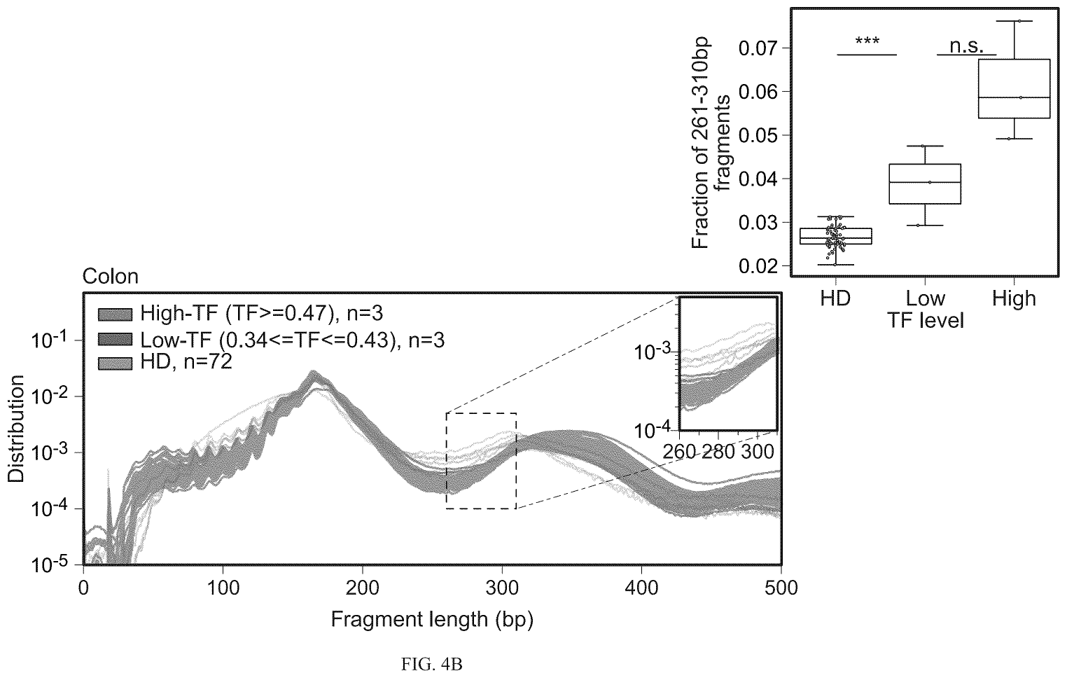

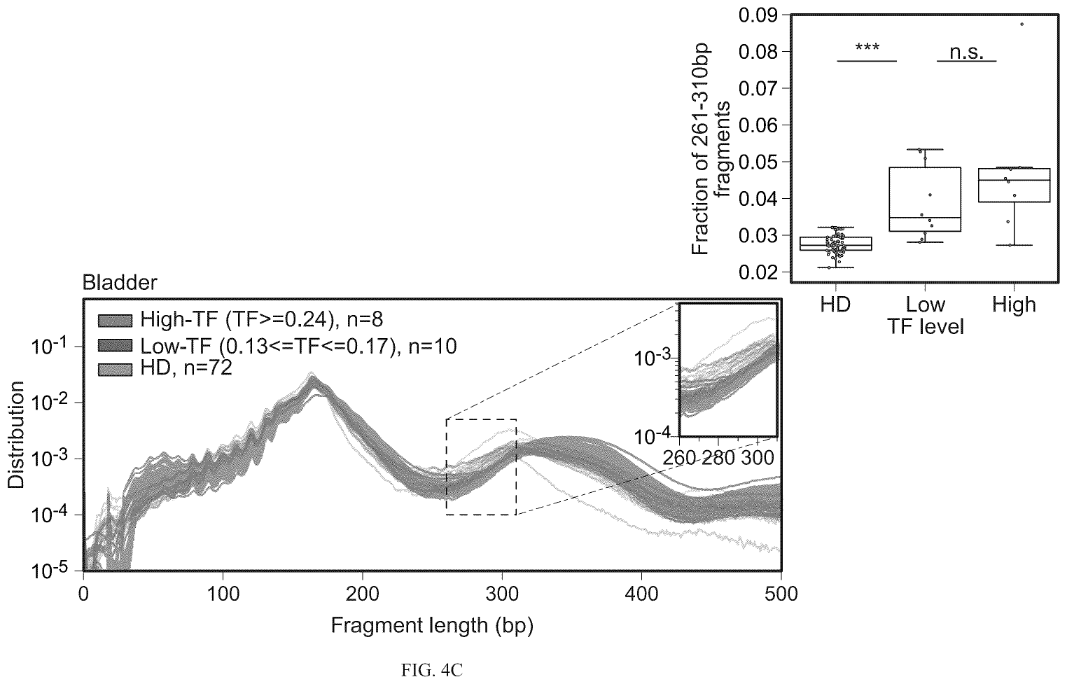

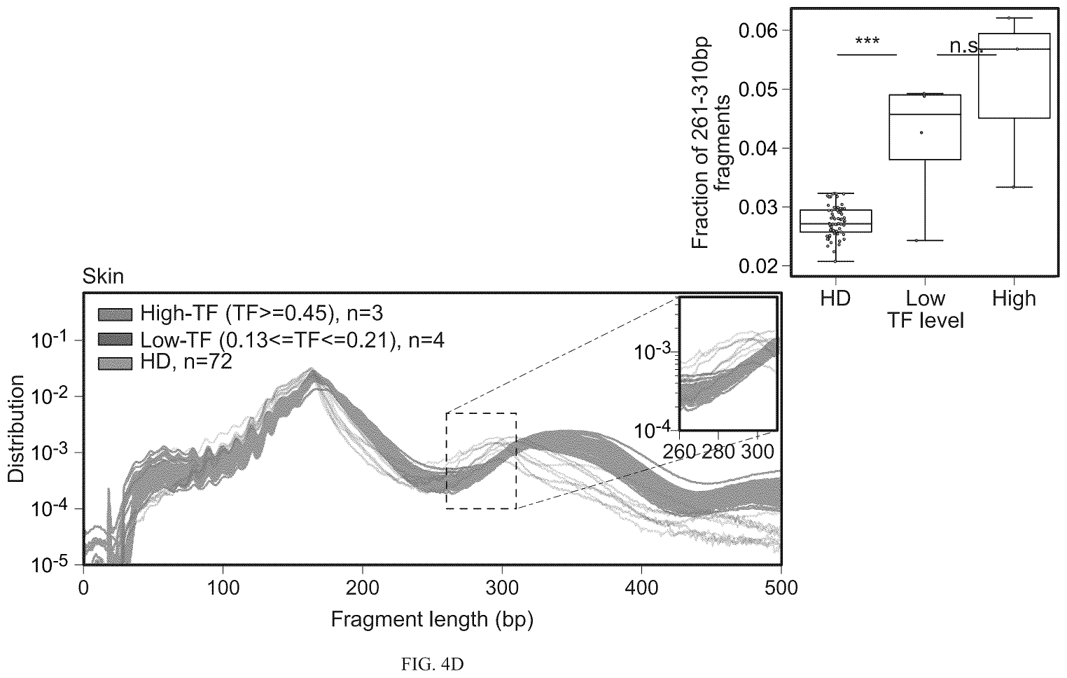

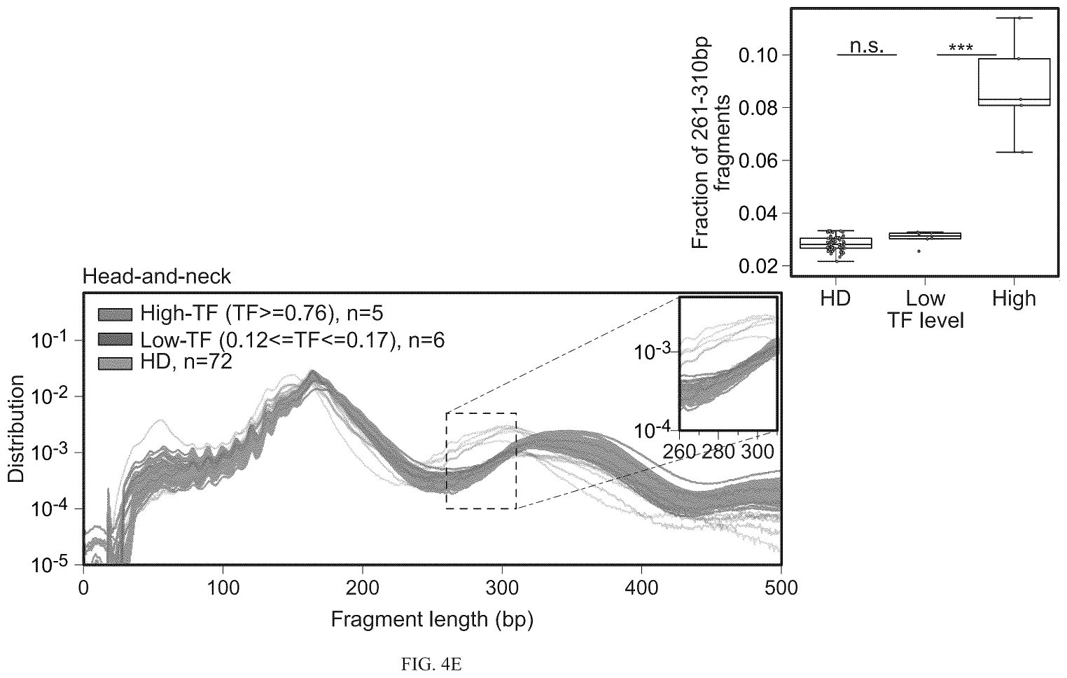

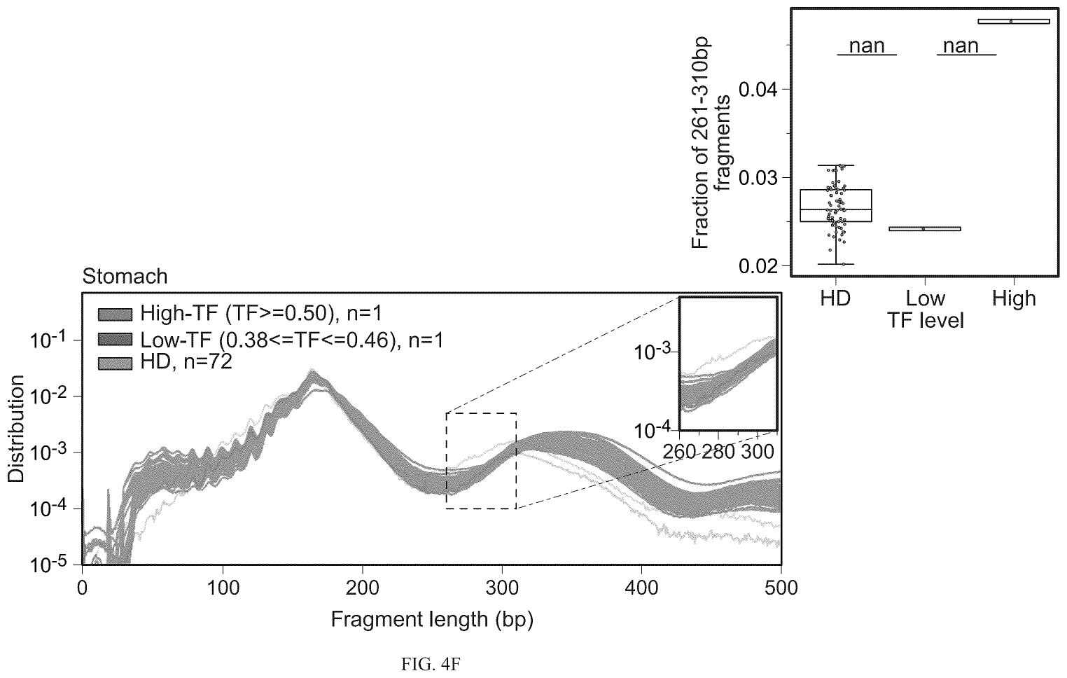

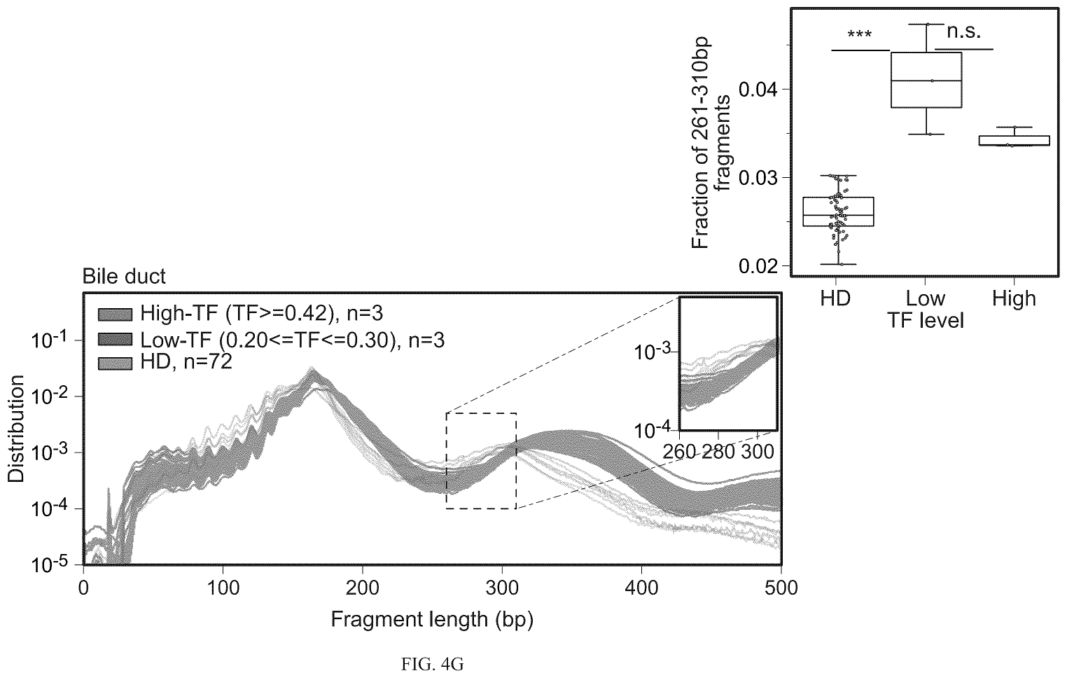

Given that cell free DNA (cfDNA) has “footprints” of nucleosome positions that inform its cell-of-origin, experiments were undertaken to compare cfDNA samples from cancer patients with high tumor fraction (high-TF) and low tumor fraction (low-TF) versus cfDNA samples from healthy donors to identify potentially altered fragment length enriched for cancer signals (FIGS. 1A-1C). Focusing on breast cancer, a significantly higher proportion of 261-310 bp fragments was observed in the high-TF cases (TF≥0.44, N=49) relative to the low-TF cases (0.18≤TF≤0.28, N=51) (mean 0.069 vs. 0.042, two-sided Student's t-test P=1.3×10-9, FIG. 1A). Consistent with these findings, a similar trend was observed when comparing the low-TF breast cancers to healthy donors (N=72: mean 0.042 vs. 0.027, two-sided Student's t-test P=8.1×10⁻²¹. FIG. 1A). To test whether these abnormalities were present in other cancer types beyond breast cancer, the same analysis was performed in seven other different cancer types-prostate; colon: bladder: skin; bile duct; stomach; and head-and-neck. Similar findings were observed across these cancer types (FIGS. 4A-4G).

To further test and confirm that the increased proportion of 261-310 bp cfDNA fragments did in fact derive from cancer cells, two significance metrics were calculated for each 10-bp fragment bin i in sample j: (i) a Signal-to-Noise Ratio (SNR_{i,j}) showing increased signal in tumors compared to normal; and (ii) leveraging the fact that the tumor DNA fraction depends on the tumor copy-number profile, the Spearman Correlation Coefficient (ρ_{i,j}) was used to assess the tumor contribution to each fragment bin proportion. Using a panel of cfDNA samples generated from healthy donors (N=72) as controls, it was shown that 281-290 bp cfDNA fragments across a cohort of breast cancer cfDNA samples with detectable cancer-specific mutations (N=194), achieved the highest average SNR (SNR₂₈₁₋₂₉₀=11; 95% confidence interval: 0.46-39), and the neighboring bins (i.e., 261-310 bp) also showed high average SNR (FIG. 1B). The same characteristic signals were observed in seven other cancer types studied (FIGS. 5A-5G). Therefore, a set containing these 5 fragment bins (261-310 bp) was defined as Y fragments. Next, for 31 cancer samples of various cancer types with significant copy number variation, a significantly positive correlation in each sample was noted between the relative cancer copy-number profile and the proportion of Y fragments across the genome: however, this correlation was completely absent in cfDNA samples from healthy donors (FIG. 1C). Not intending to be bound by theory, taken together, the data suggest that the increased proportion of cfDNA fragments in Y can reliably detect the presence of a tumor across various tumor types. Rather than using bins corresponding to smaller fragment sizes, the longer Y fragments were used because they were found to have a high SNR (FIG. 1B).

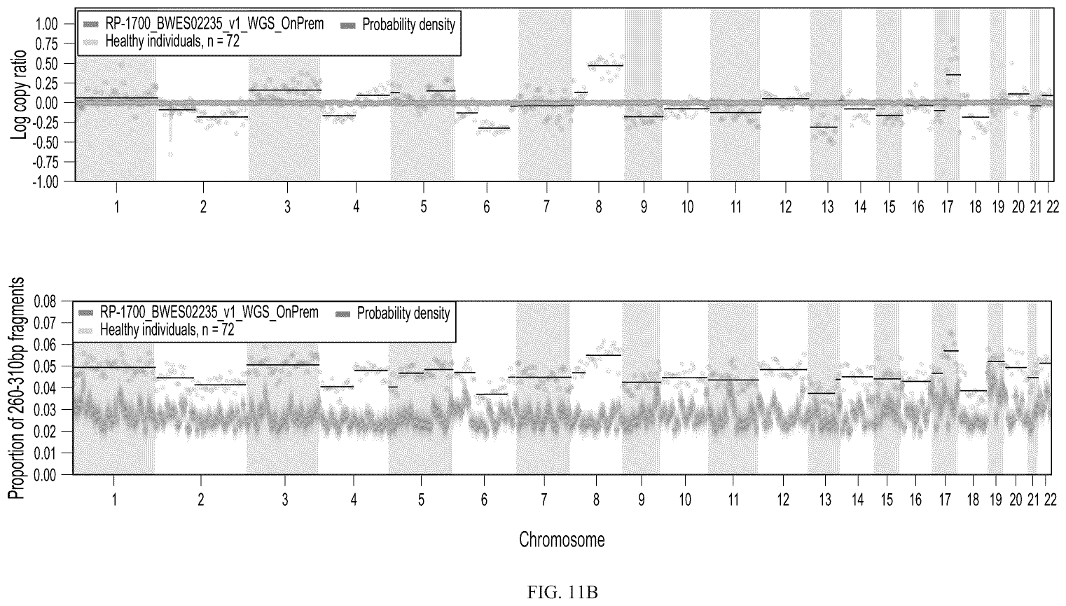

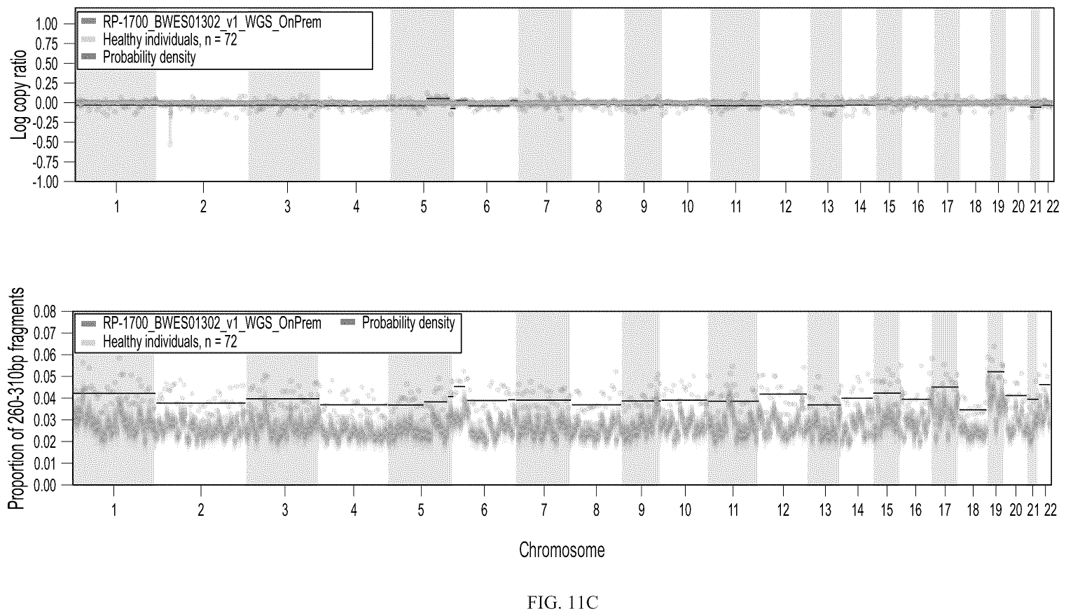

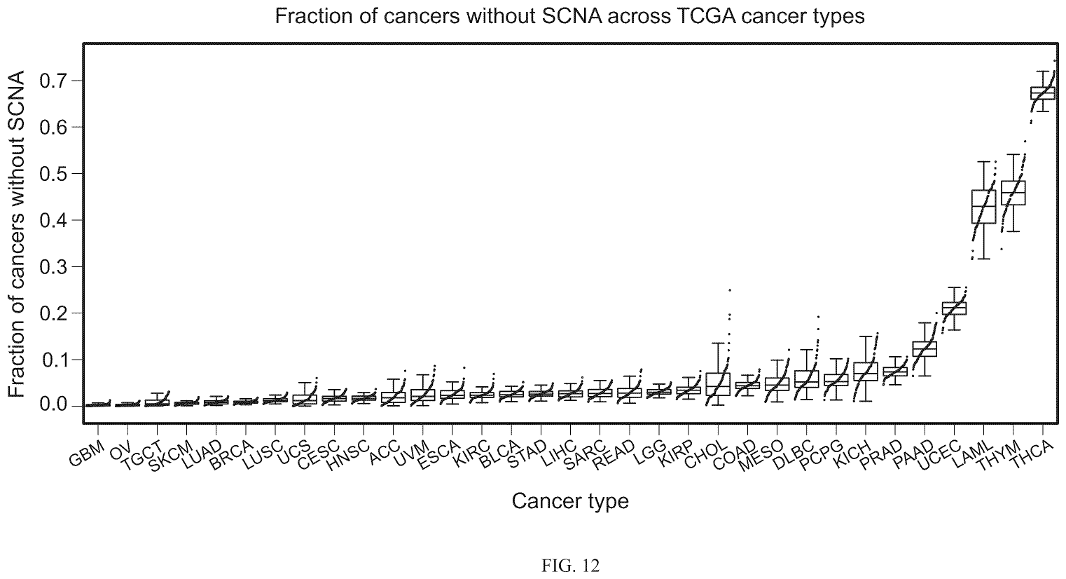

Moreover, methods estimating tumor fraction (TF) exclusively based on somatic copy number alterations (SCNAs) can lose tumor signal in either copy number-quiet tumors or tumors dominated by copy-neutral loss-of-heterozygosity. Using 9,613 TCGA SNP array data, it was found that even for high-TF cancers (TF≥20%), approximately 7.2% did not have clear SCNA signals, with some cancer types having extremely high fractions of copy number-quiet tumors (e.g., 68% in thyroid carcinoma) (FIG. 12). Interestingly, leveraging both SCNA and altered fragment length, rather than using either feature by itself, provided a synergistic effect through orthogonal constraints that complemented each other and together achieved a higher sensitivity for detecting cancer. Indeed, out of 9 cfDNA samples with validated cancer mutations from a single breast cancer patient, only 6/9 had SCNA signals, while all 9/9 had either SCNA or altered Y fragment signals. The complementary benefits of considering both SCNAs and Y fragments were also shown using three independent cfDNA samples as further examples (FIGS. 11A-11C). Additionally, given the difficulty in many cases to distinguish clonal from sub-clonal copy-number events, which is required for accurate tumor fraction (TF) estimation, the fragment size information used by the method provided herein (i.e., TuFEst) provides additional constraints in the search for possible TF values.

### Example 2: Tumor Fraction Estimator (TuFEst)

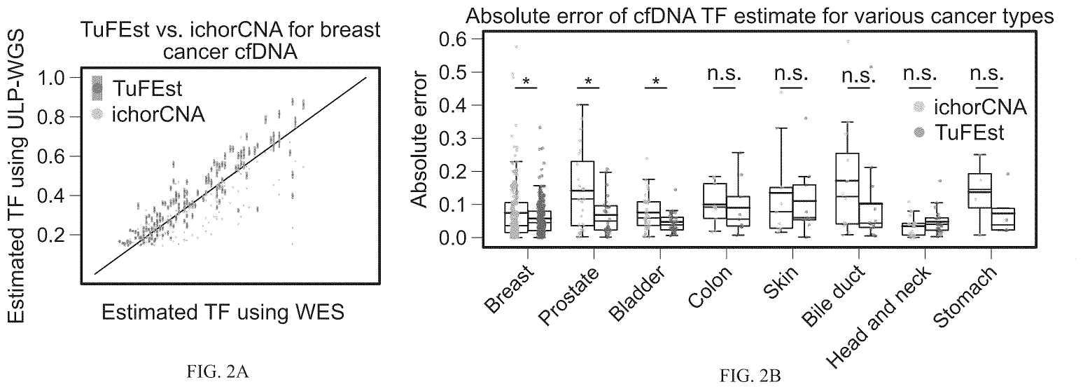

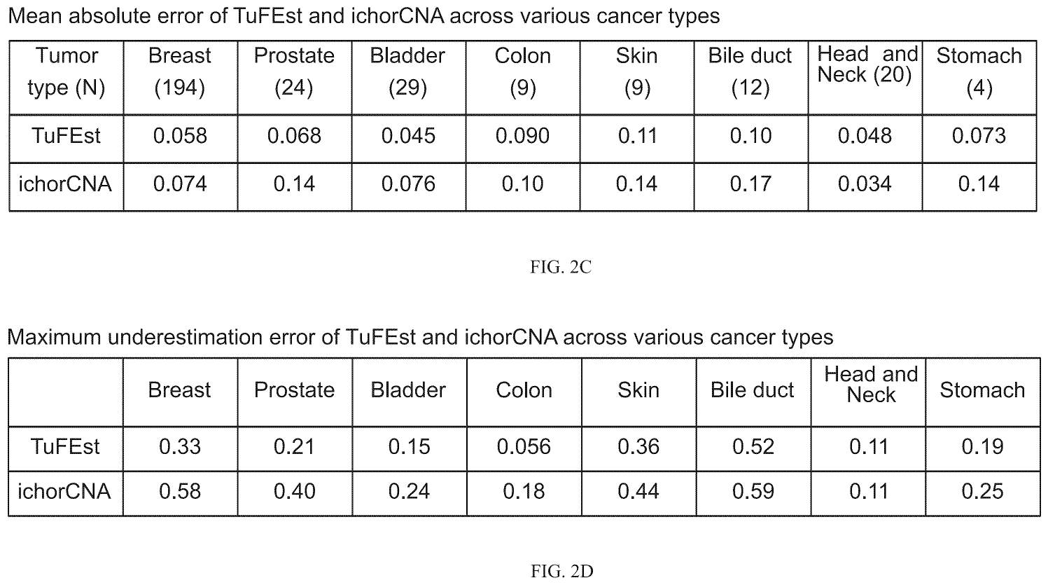

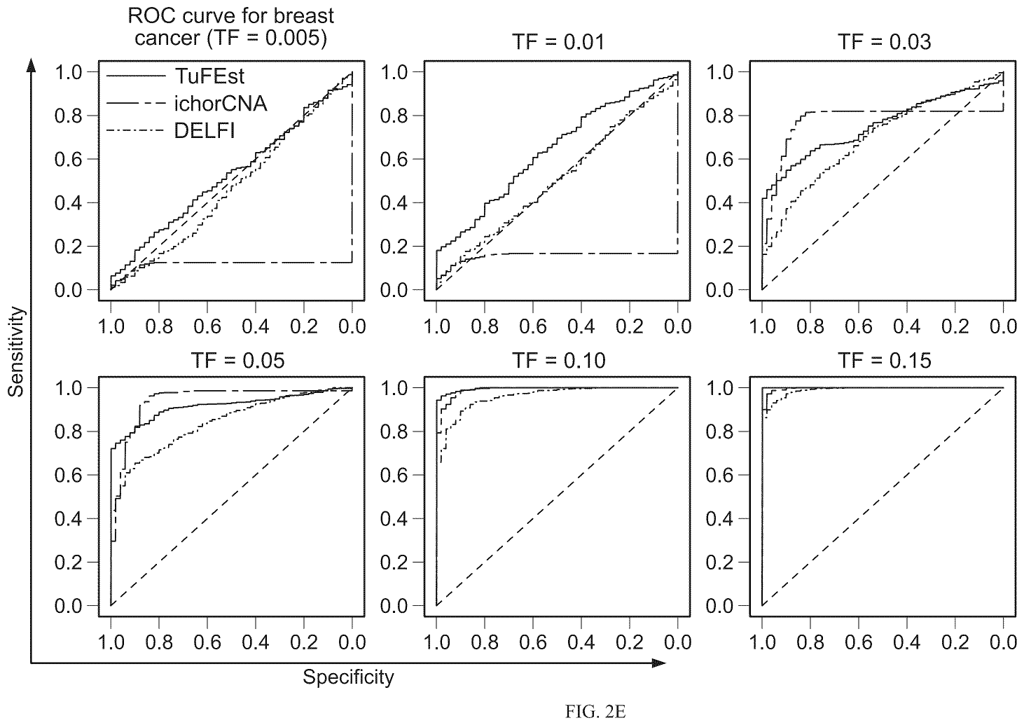

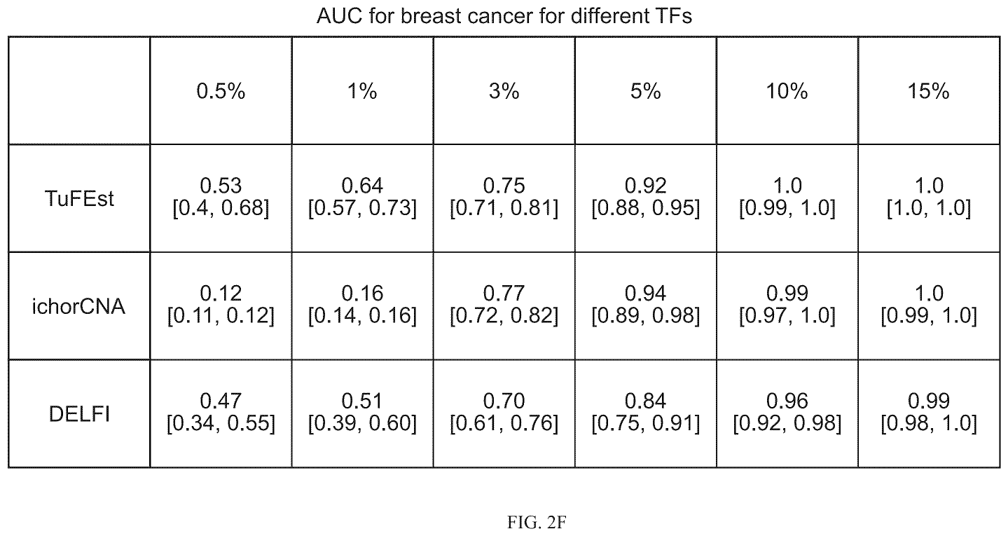

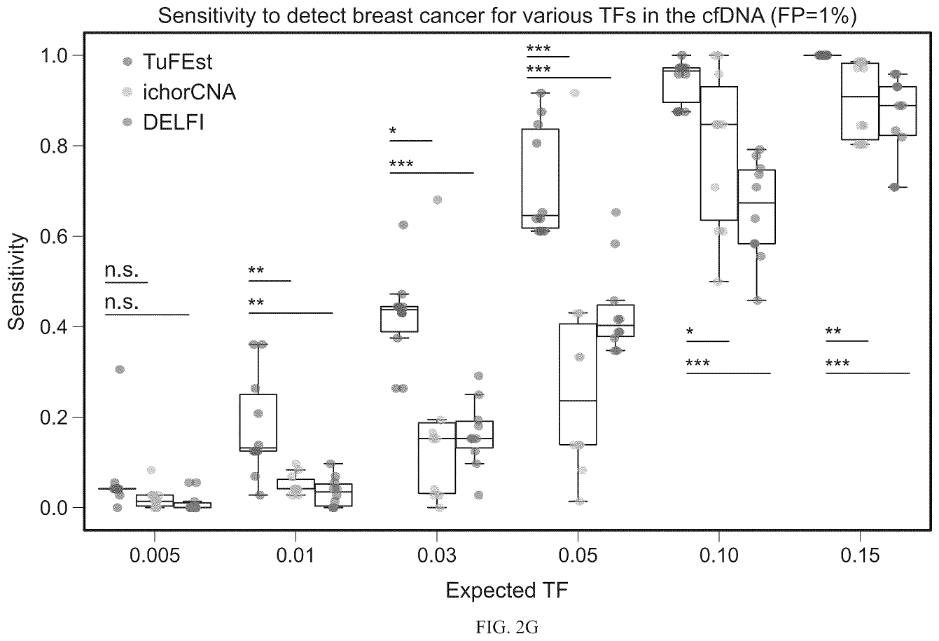

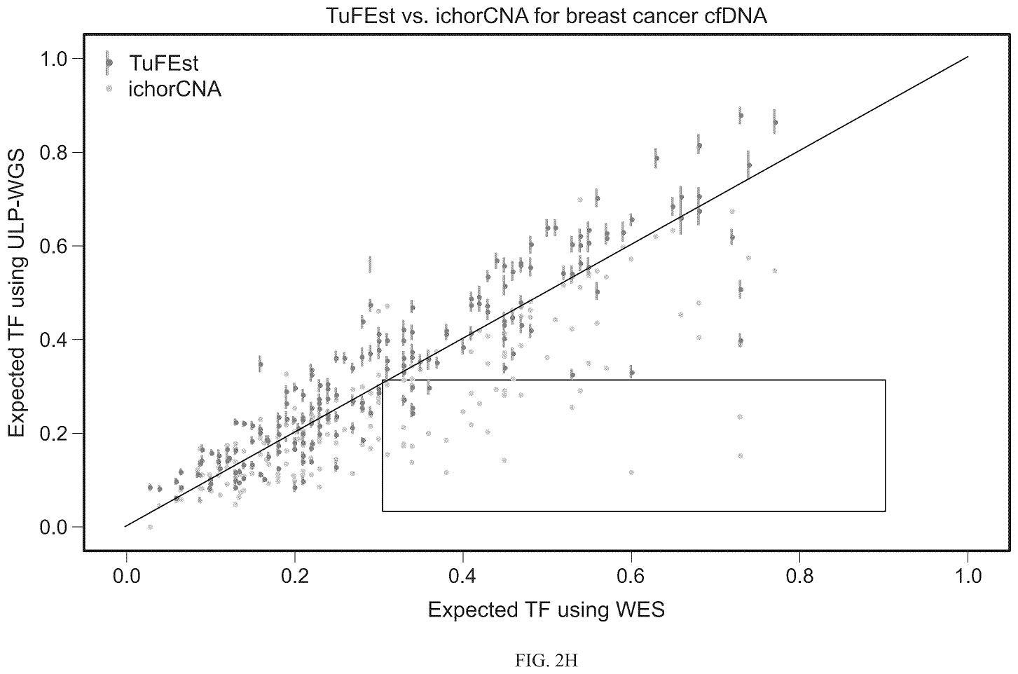

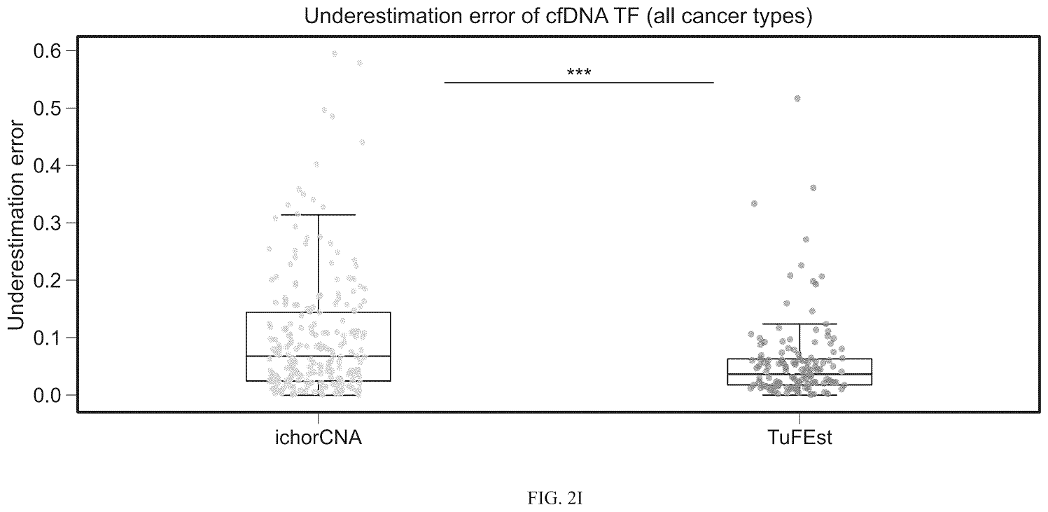

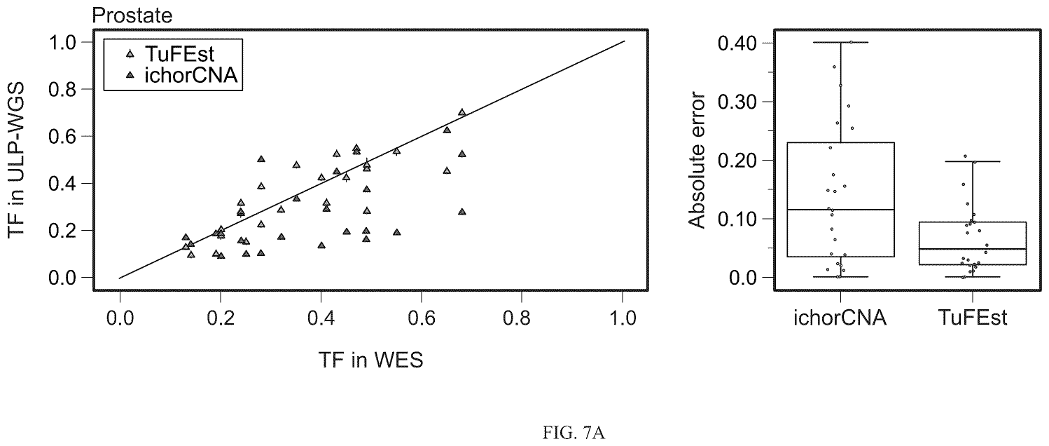

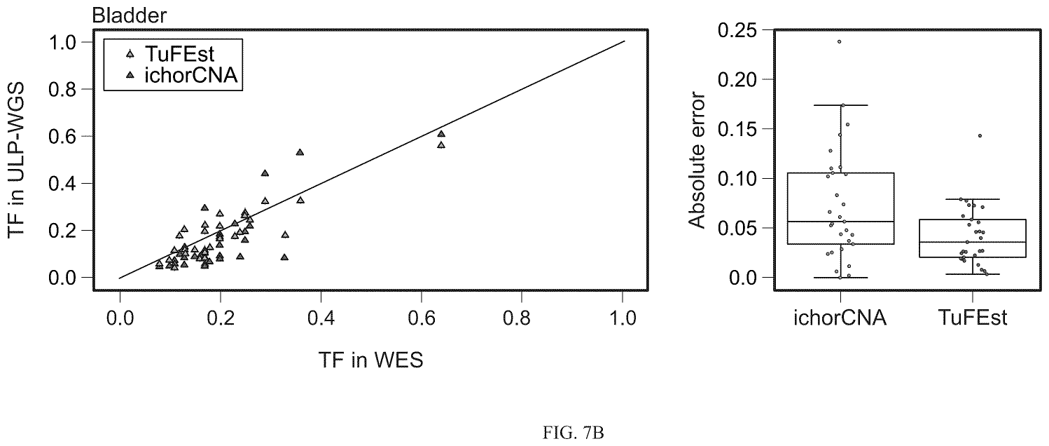

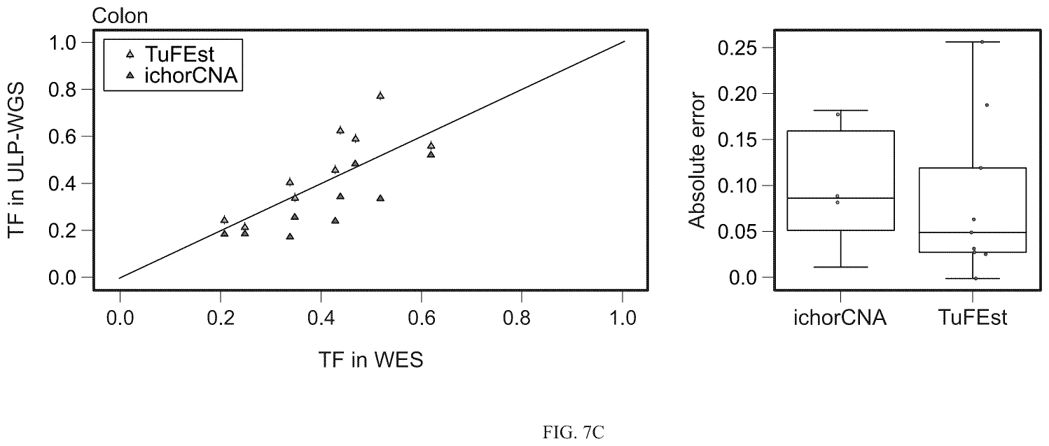

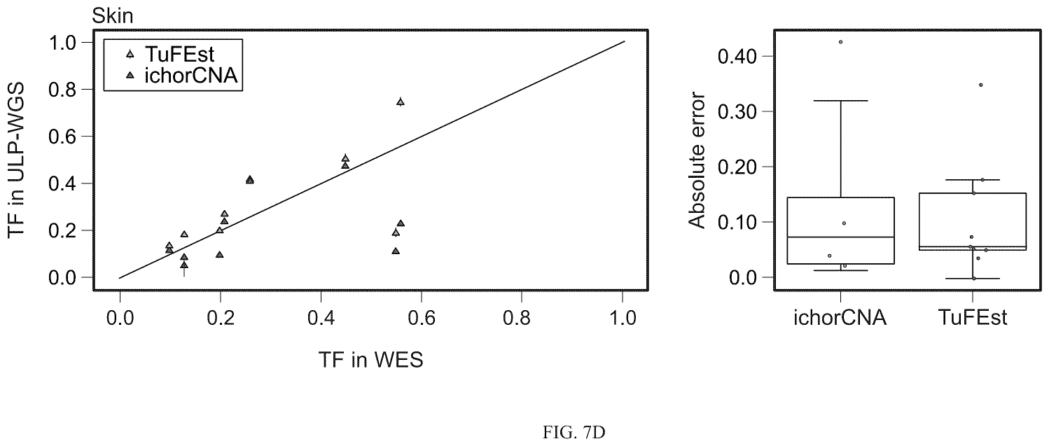

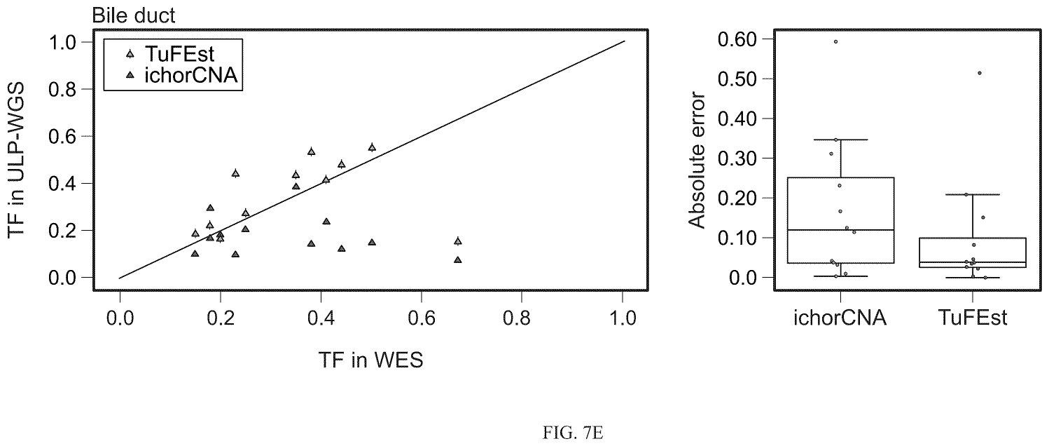

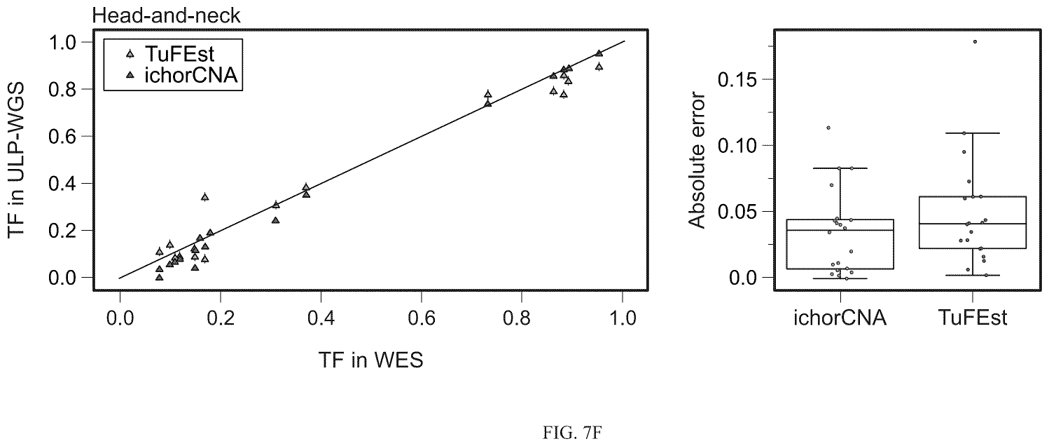

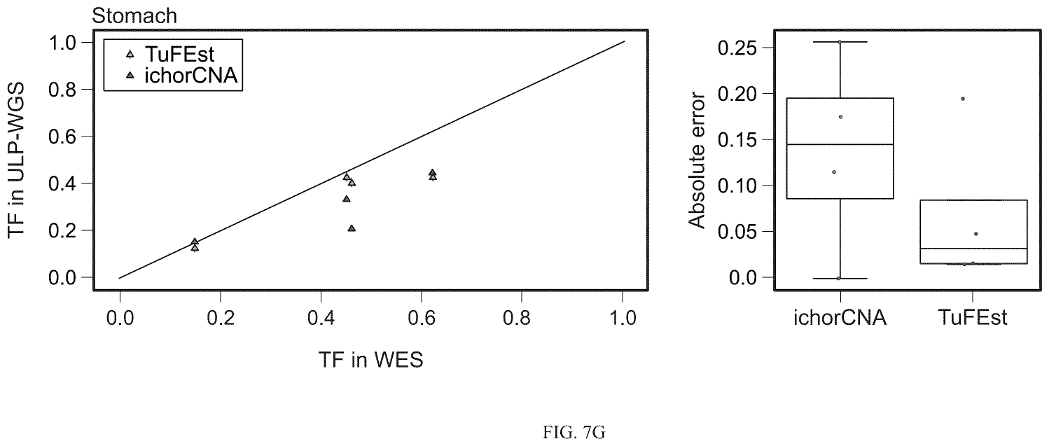

Given the observation presented in Example 1 above of tumor derived DNA in cfDNA being enriched in the Ψ fragments, a Bayesian-based method called TuFEst (Tumor Fraction Estimator) was developed to improve TF estimation by combining information from the Ψ fragments and copy number alterations. TuFEst used a Bayesian approach, in which the evidence and uncertainties from both sources of data (i.e., Ψ fragments and SCNAs) was integrated to produce a joint posterior distribution over the TF values and the predicted total copy-number profile, from which the marginal posterior distribution over the TF values was extracted. In order to begin evaluating TuFEst's ability to estimate TF, TuFEst was first implemented on ultra-low pass whole-genome sequencing (ULP-WGS) data (median: 0.24× coverage: range: 0.055-3.4× coverage) of cfDNA samples from 301 cancer patients representing eight different cancer types and compared it against gold standard results from ABSOLUTE (Carter, Scott L., Kristian Cibulskis, Elena Helman, Aaron Mckenna, Hui Shen, Travis Zack, Peter W. Laird, et al. 2012. “Absolute Quantification of Somatic DNA Alterations in Human Cancer.” Nature Biotechnology 30 (5): 413-21) based on whole exome sequencing (WES) data (˜150× coverage) derived from the same samples (FIGS. 2A and 7A-7G). It was observed that the tumor fraction (TF) from these real, cancer patient-derived cfDNA samples had a wide range, from <3% to >95%, and that the estimated TF closely followed the expected TF for most patients (range of mean absolute error per tumor type: 4.5%-11%: FIGS. 2A and 7A-7G).

To benchmark the performance of TuFEst against a widely used method for estimating TF from ULP-WGS, TuFEst was compared to ichorCNA (Adalsteinsson, Viktor A., Gavin Ha, Samuel S. Freeman, Atish D. Choudhury, Daniel G. Stover, Heather A. Parsons, Gregory Gydush, et al. 2017. “Scalable Whole-Exome Sequencing of Cell-Free DNA Reveals High Concordance with Metastatic Tumors.” Nature Communications 8 (1): 1324.). TuFEst and ichorCNA were implemented on the same cell-free DNA (cfDNA) samples representing various cancer types. On average, TuFEst achieved significantly better accuracy over ichorCNA in 3/8 cancer types (Benjamini-Hochberg corrected two-sided Student's t-test W=0.072, 0.045, 0.045 for breast, prostate, bladder, respectively), while the performance gain was not significant for the other 5 cancer types, likely due to limited sample sizes (Benjamini-Hochberg corrected two-sided Student's t-test Q=0.78, 0.78, 0.48, 0.48, 0.48 for colon, skin, bile duct, head-and-neck, and stomach, respectively) (FIGS. 2A, 2D, and 2E and 7A-7G). In both methods, it was observed that tumor fraction (TF) over-estimation occurred less frequently than under-estimation (FIGS. 2A, 2D, and 2E). TF under-estimation could have more severe clinical implications, since missing the presence of tumor burden might prevent a clinical switch to a more effective therapy for a patient. Therefore, the maximum (and median) under-estimated case in each tumor type was compared and it was found that TuFEst exhibited less TF under-estimation than ichorCNA (average maximum [median] severe under-estimation across tumor types was 24% [4.3%] for TuFEst and 35% [10%] for ichorCNA: FIGS. 2A and 7A-7G).

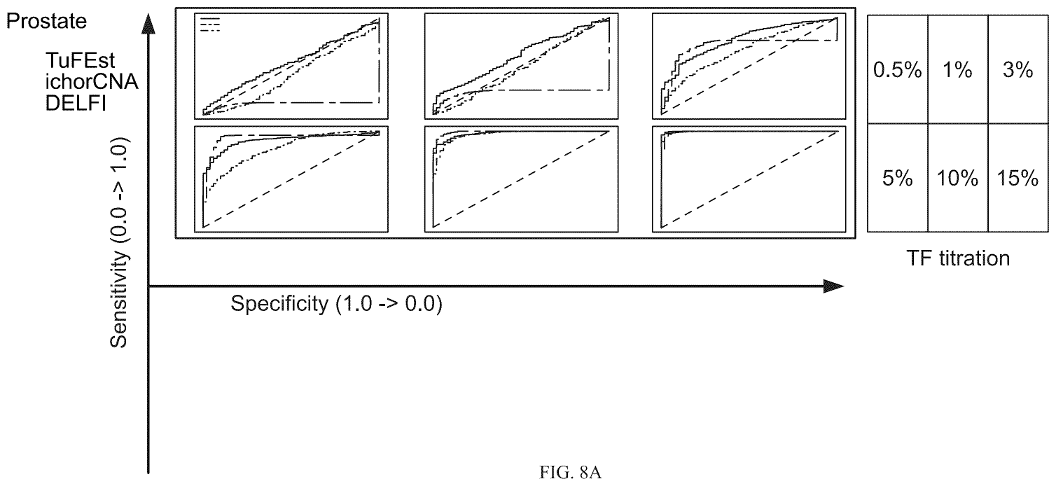

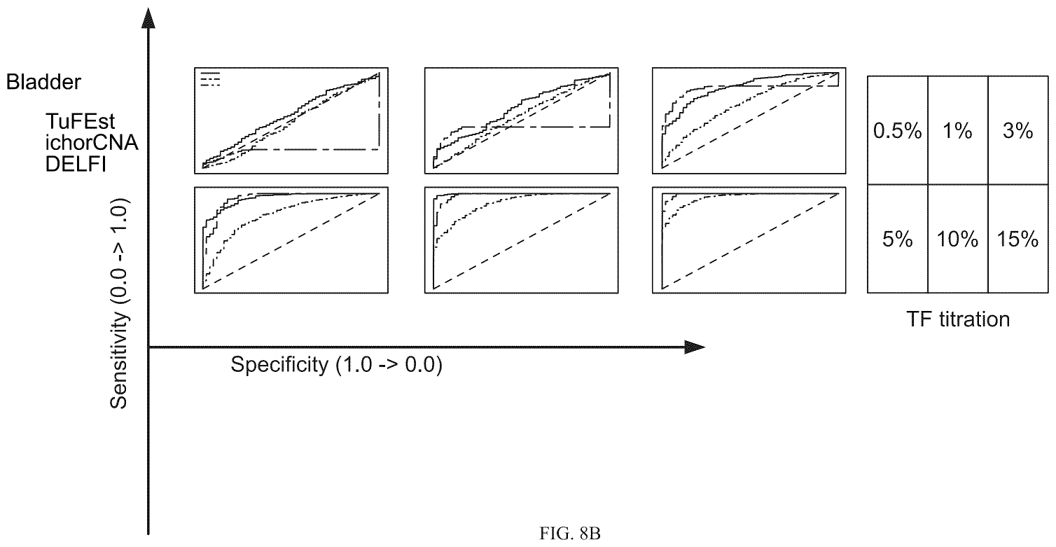

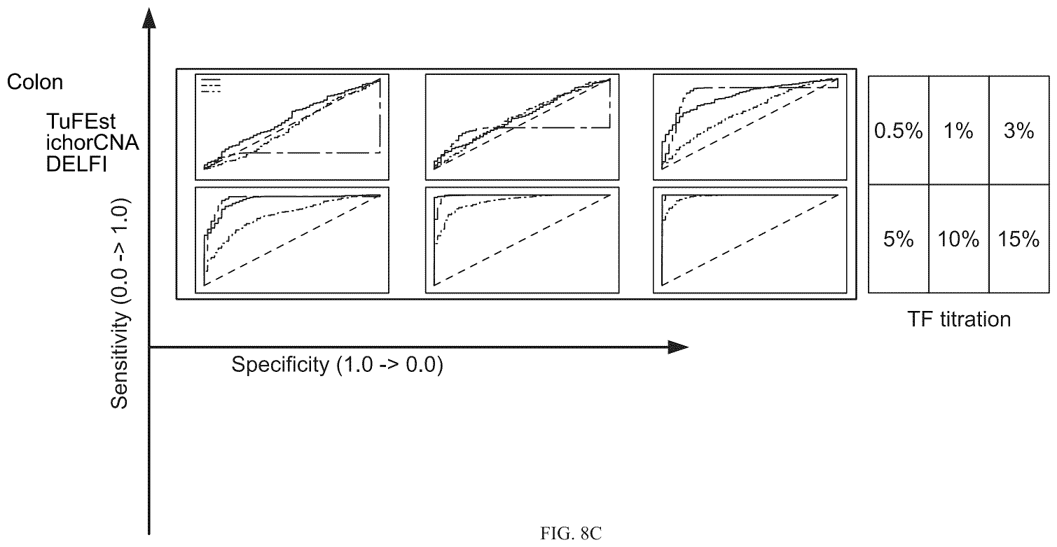

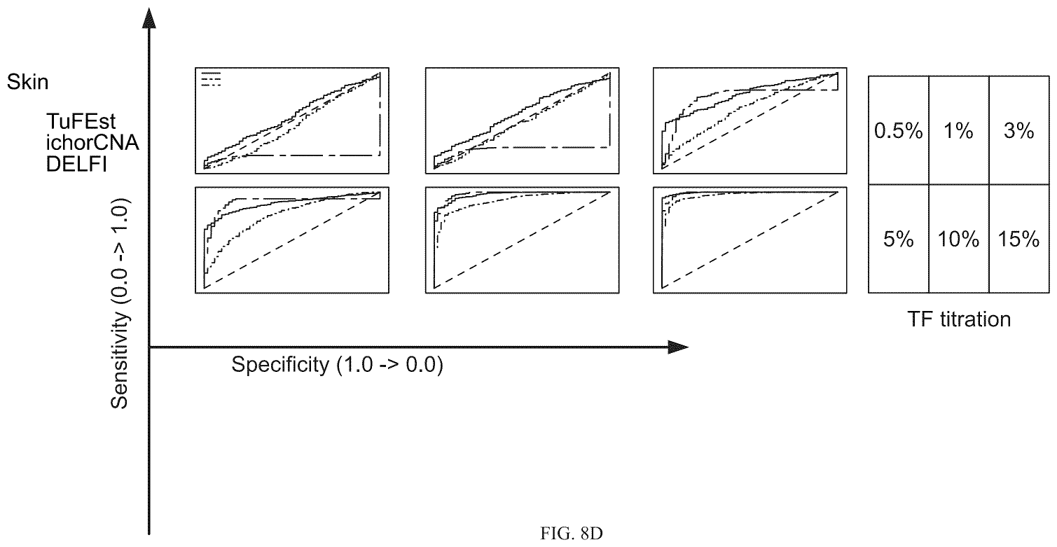

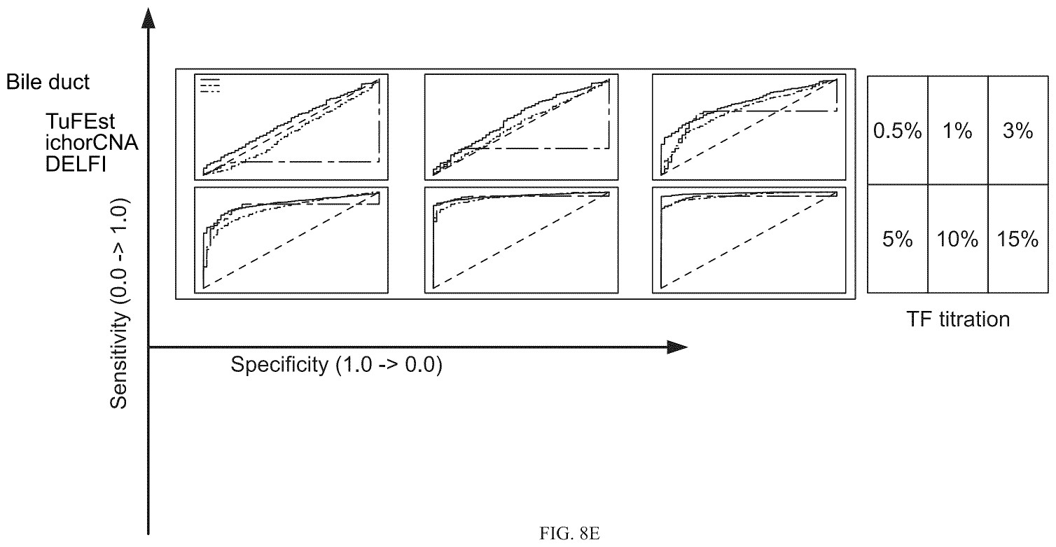

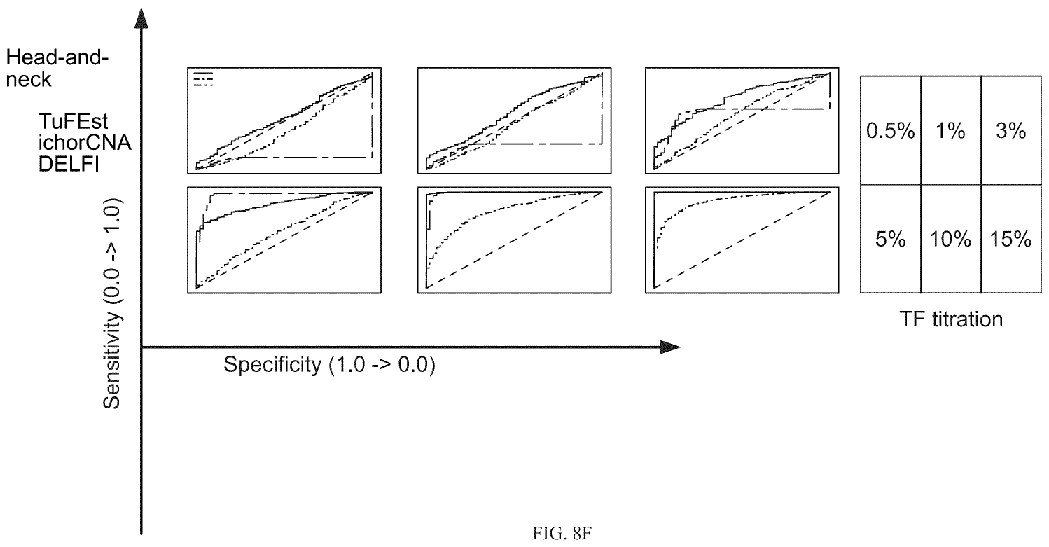

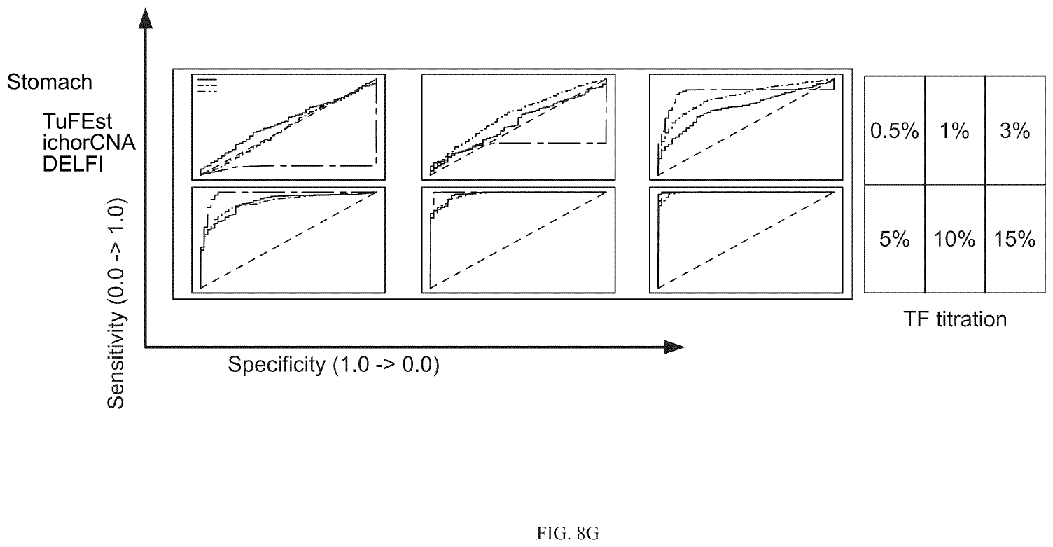

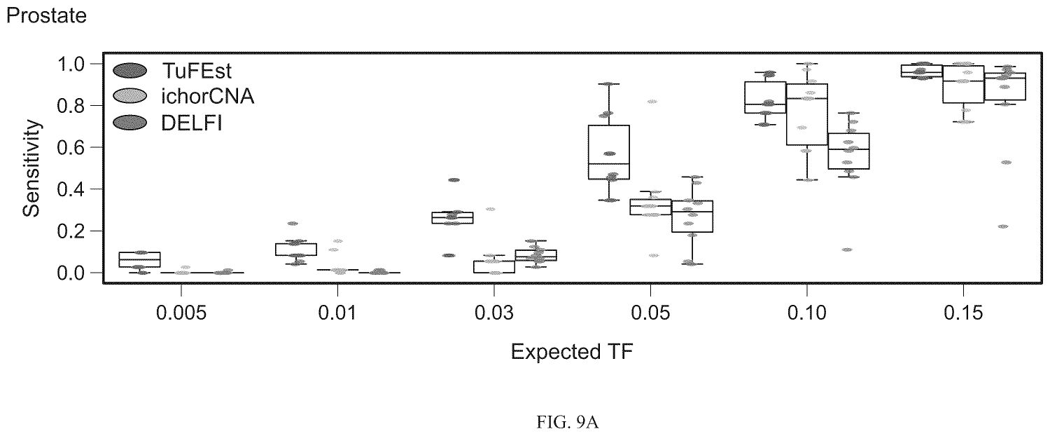

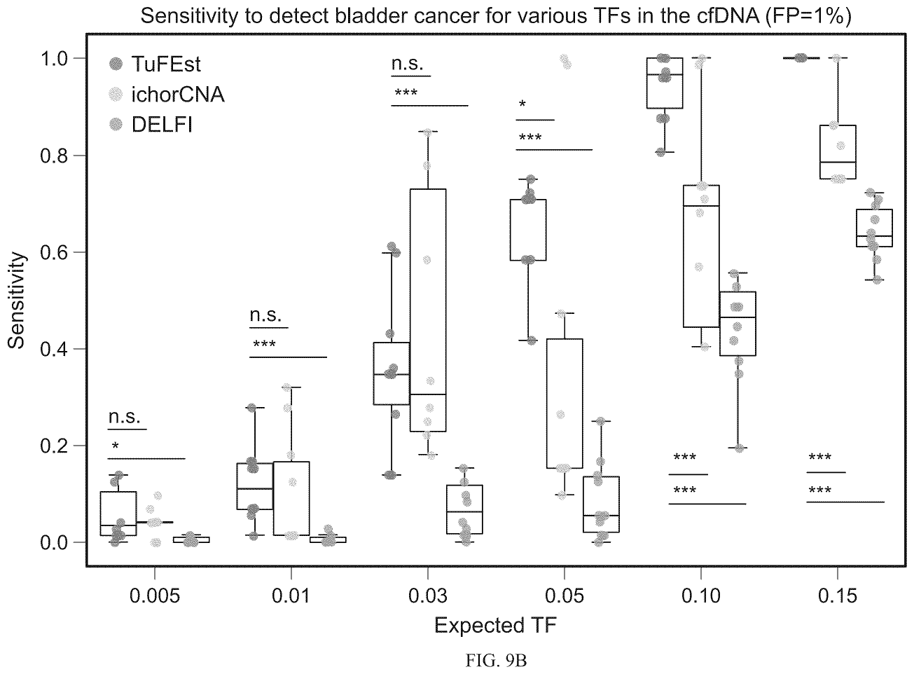

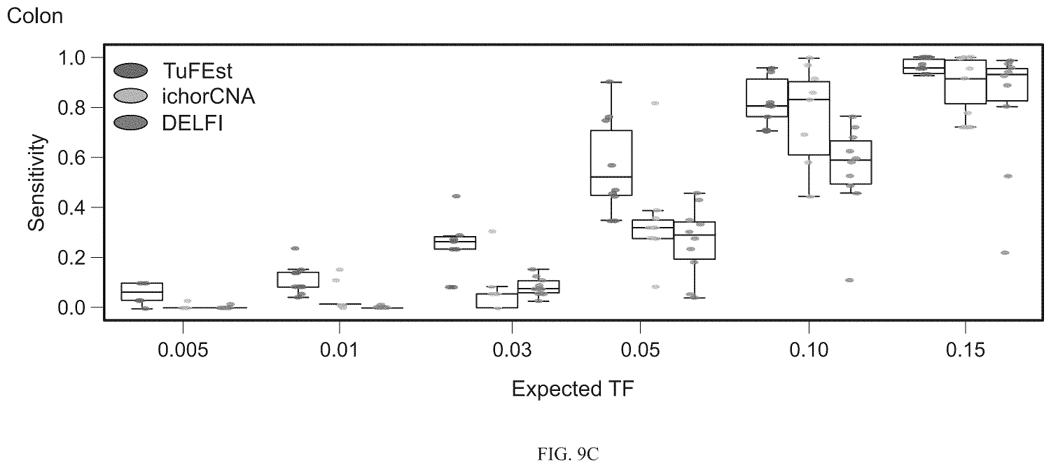

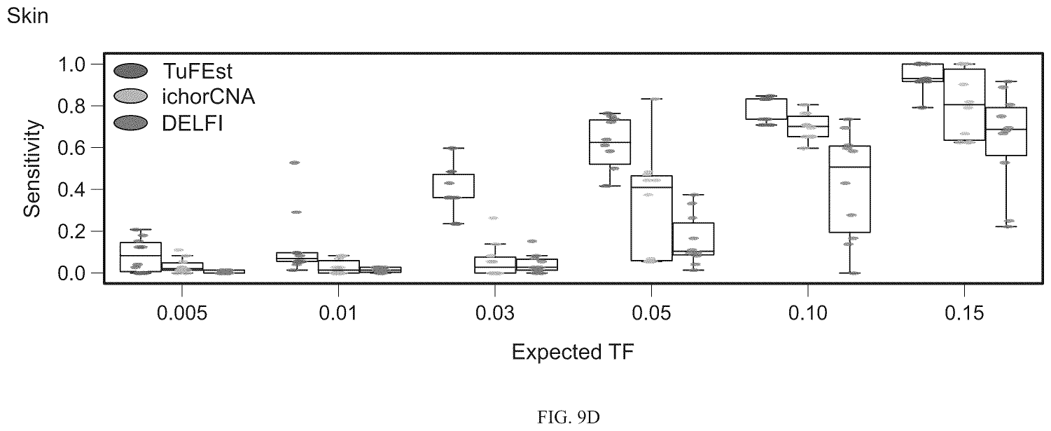

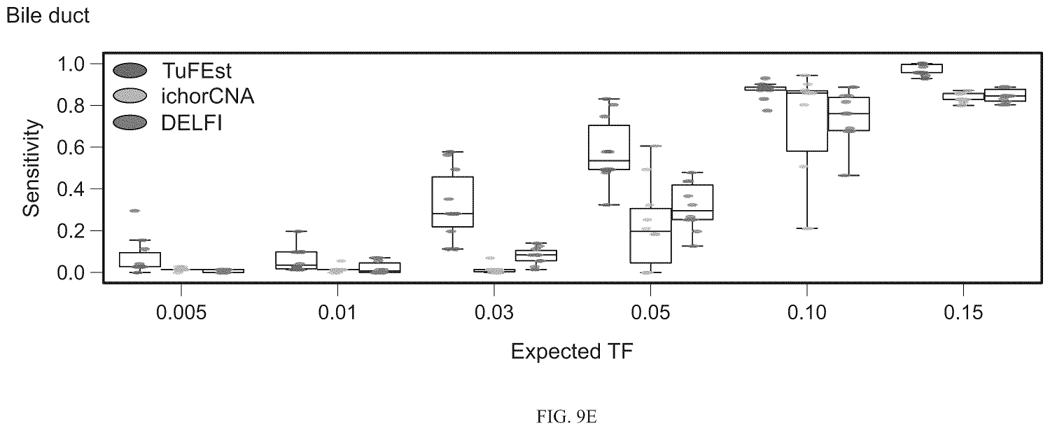

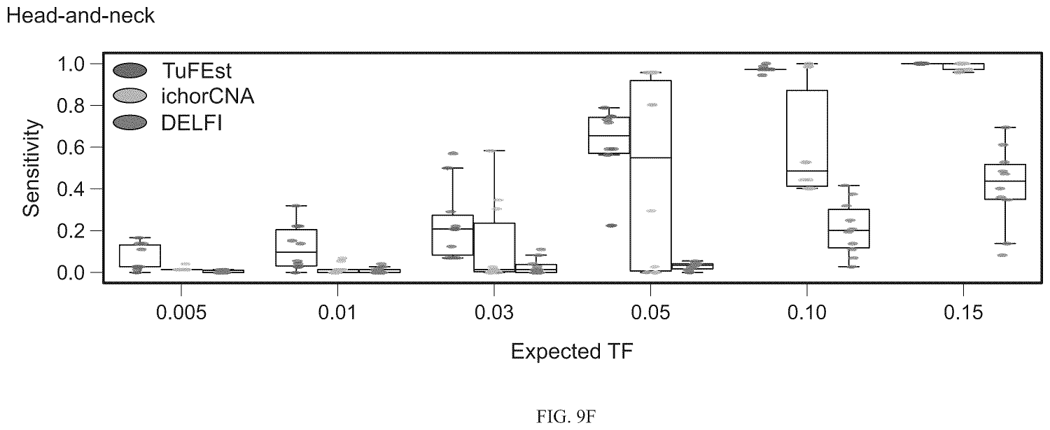

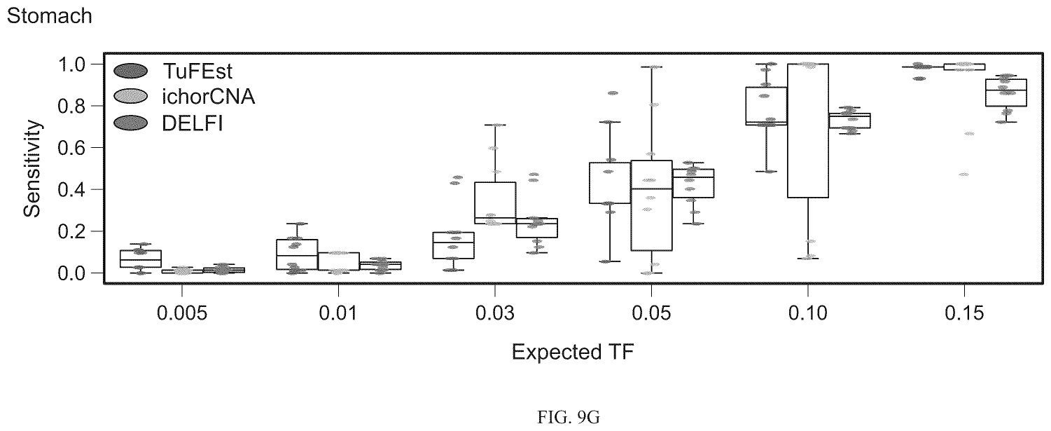

The performance of TuFEst was next tested against another additional method called DELFI (Cristiano, Stephen, Alessandro Leal, Jillian Phallen, Jacob Fiksel, Vilmos Adleff, Daniel C. Bruhm, Sarah Østrup Jensen, et al. 2019. “Genome-Wide Cell-Frec DNA Fragmentation in Patients with Cancer.” Nature 570 (7761): 385-89), a machine learning (ML)-based classifier that uses fragment length information to classify samples as either cancerous or normal/healthy. To test the performance of the two methods across different tumor fractions (TFs) and cancer types, for each cancer type, 432 in-silico cancer ULP-WGS data was generated by mixing high TF cfDNA data from cancer patients with cfDNA data from 72 independent healthy donors in silico, such that 6 different TF values were obtained (72 mixes per TF value). To generate 360 healthy donor cfDNA data, each of the 72 healthy donor datasets that were sequenced to higher coverages (median: 3.5×: range: 1.6-21×) were down-sampled, and 5 ˜0.2× cfDNA data sets were generated to match the depth of the cancer patient samples. Since DELFI required training, it was trained on cfDNA from 360 cancer patients and 310 of the 360 down-sampled healthy donor cfDNA data, and it was then tested on the in-silico cancer mixtures and the 50 remaining down-sampled healthy donors. To ensure consistency, all methods evaluated (TuFEst, DELFI and ichorCNA) were tested on the exact same data sets (FIGS. 2B and 8A-8G). The detection accuracy of TuFEst increased monotonically with tumor fraction (TF) (FIG. 2B, TF=0.5%, mean area under the receiver operating characteristic (ROC) curve (AUC)=0.53: TF=3%, AUC=0.75; TF=5%, AUC=0.92: TF=10%, AUC=1.0). Furthermore, TuFEst achieved significantly higher AUC in detecting low TF breast cancer than ichorCNA (e.g., TF=0.5%, 1%), with comparable AUC in cases with TFs >3%. These findings were consistent in all seven other cancer types (FIG. 8A-G). TuFEst also consistently outperformed DELFI in a direct comparison study across all TFs in breast cancer (FIG. 2B). This finding was also consistent in the majority of testing scenarios in the seven other cancer types, other than in stomach cancer with TFs between 1-3% (FIGS. 8A-8G). Given the importance of minimizing the false-positive (FP) rate in early cancer screening, sensitivity was also compared across the three methods by setting the FP rate to 1%. Overall, TuFEst showed higher median sensitivity than the other two methods in about 88% of testing scenarios across all eight cancer types (FIGS. 2C and 9A-9G).

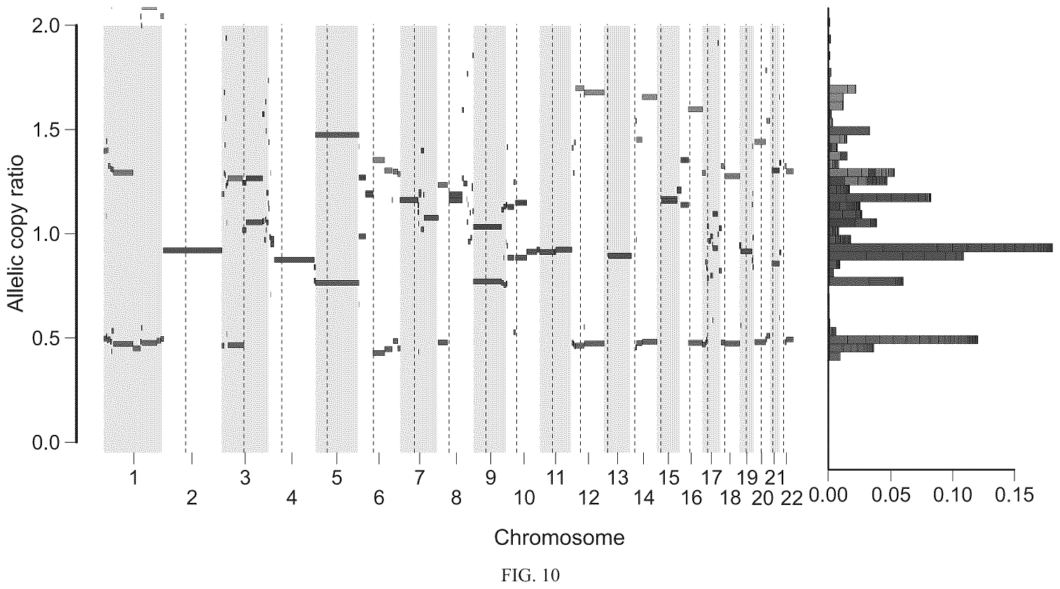

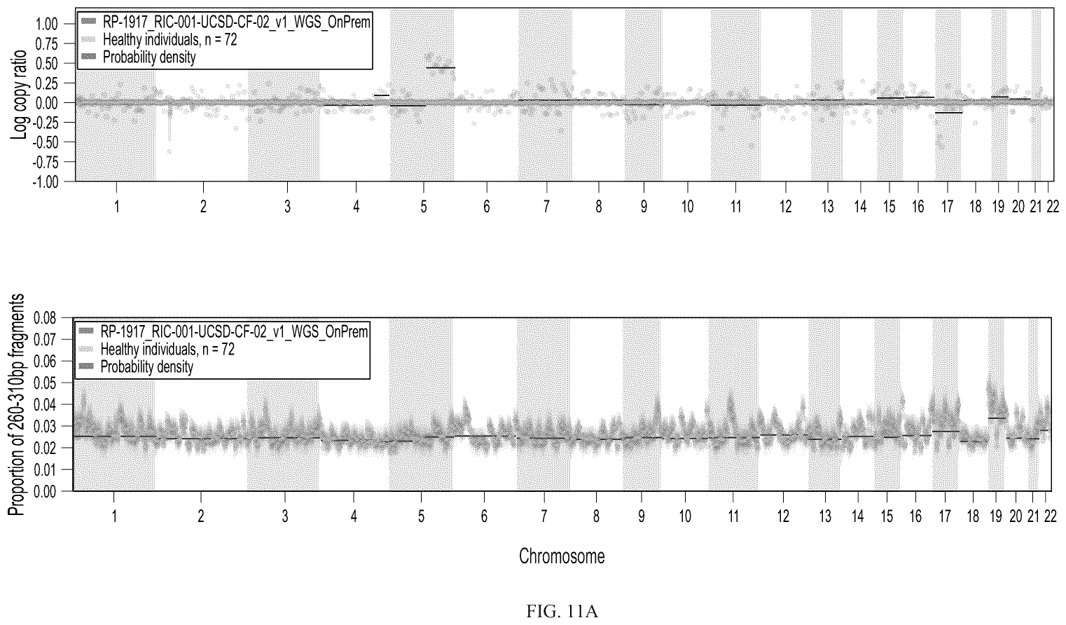

To further assess TuFEst's detection sensitivity, ˜300× whole-exome sequencing (WES) data from 9 serial cfDNA samples from a single breast cancer patient was also analyzed, for which the existence of cancer DNA in the cfDNA was validated by cancer mutations seen in solid biopsies from the same patient. Again, by setting the false-positive rate (FP) threshold at 1% using 360 down-sampled healthy samples of matching depth (˜0.2×), TuFEst successfully detected cancer in 8 samples (8/9-88.9%), while ichorCNA failed to detect cancer in any of the serial cfDNA samples (0/9) with confirmed cancer DNA (FIG. 10). Overall, since TuFEst directly modeled the effects of tumor fraction (TF) on read count data, which reflects copy-number alterations, as well as fragment length distribution, it could achieve higher accuracy with a relatively small training data set. Not intending to be bound by theory, this is likely due to fewer parameters to fit, and that the relationship between the parameters of the model reflect their true biological relationships. The performance of any method that uses cfDNA data to predict cancer vs. healthy donors is expected to increase with TF, as observed for TuFEst. Cases with no tumor DNA (i.e., TF=0%, due to the effectiveness of treatment or in cured cancer patients) should not be detected as cases with cancer and should not be used for training as cancer samples.

### Example 3: Increasing Tumor Fraction Estimator (TuFEst) Accuracy

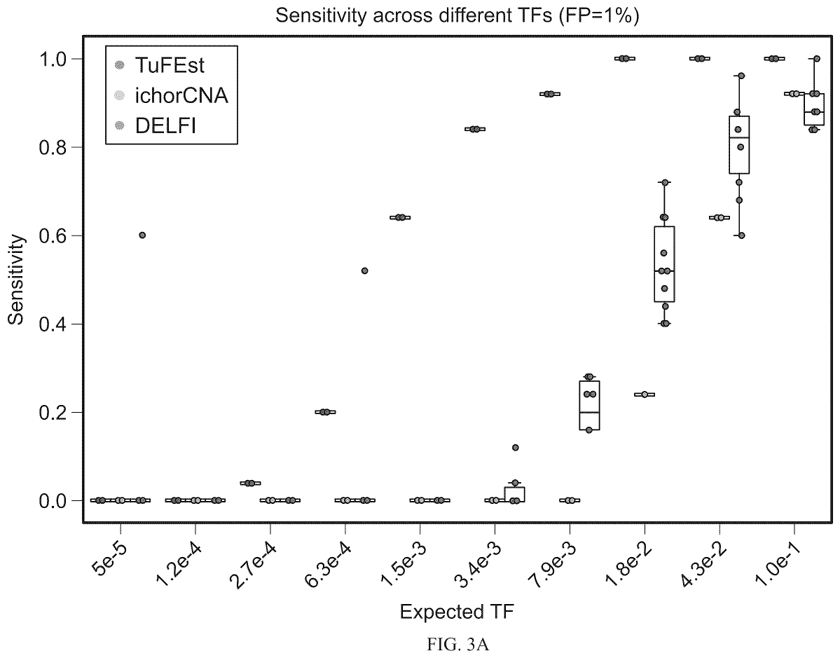

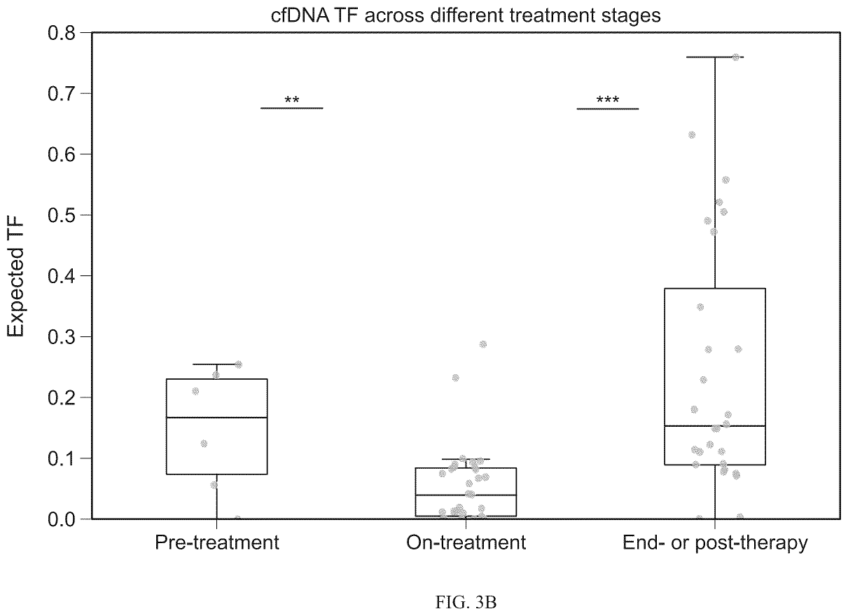

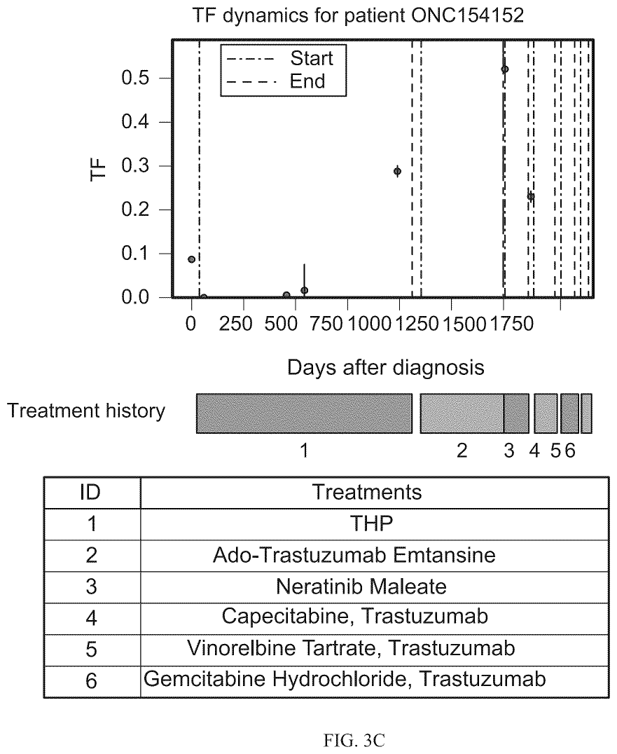

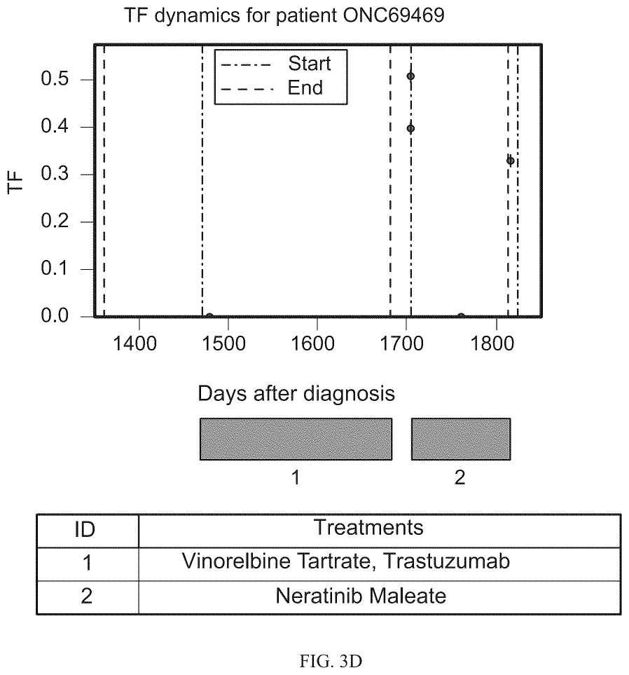

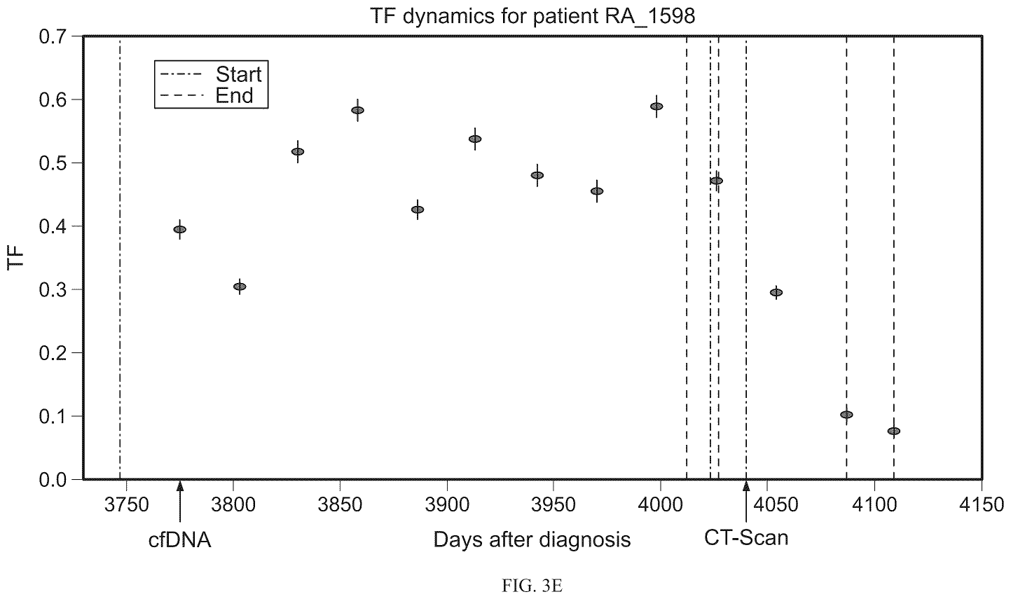

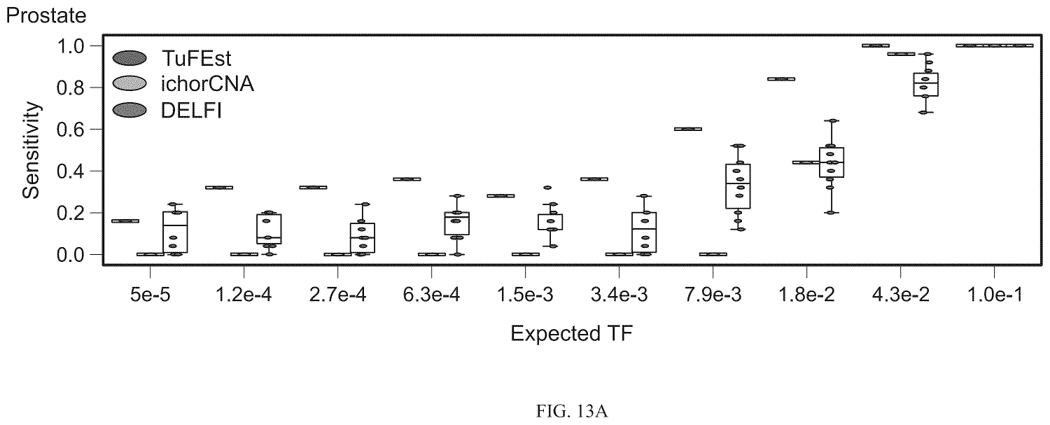

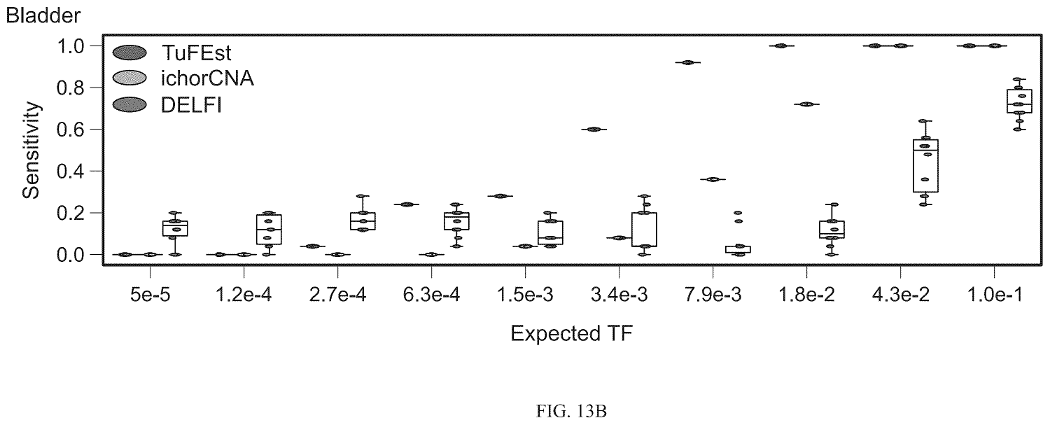

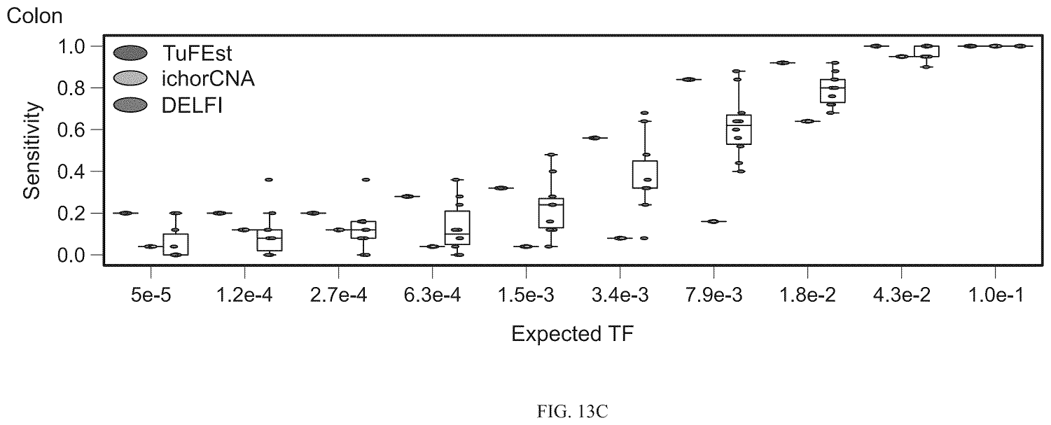

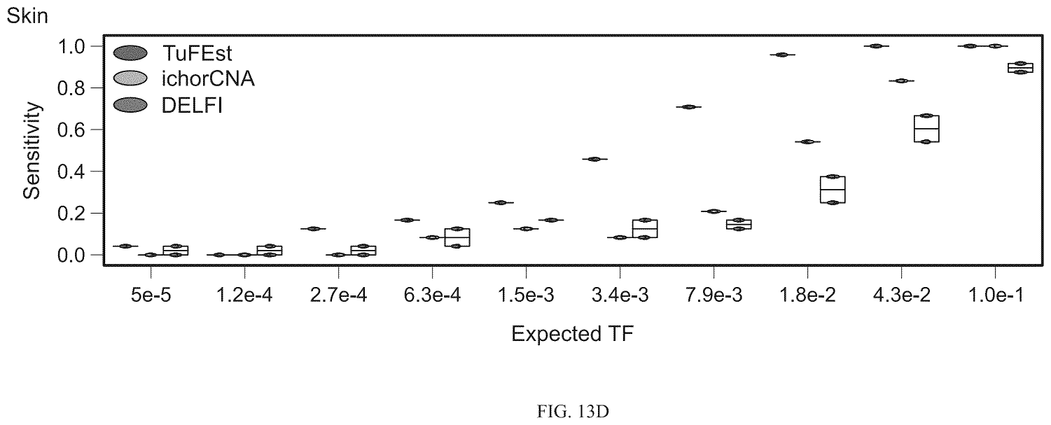

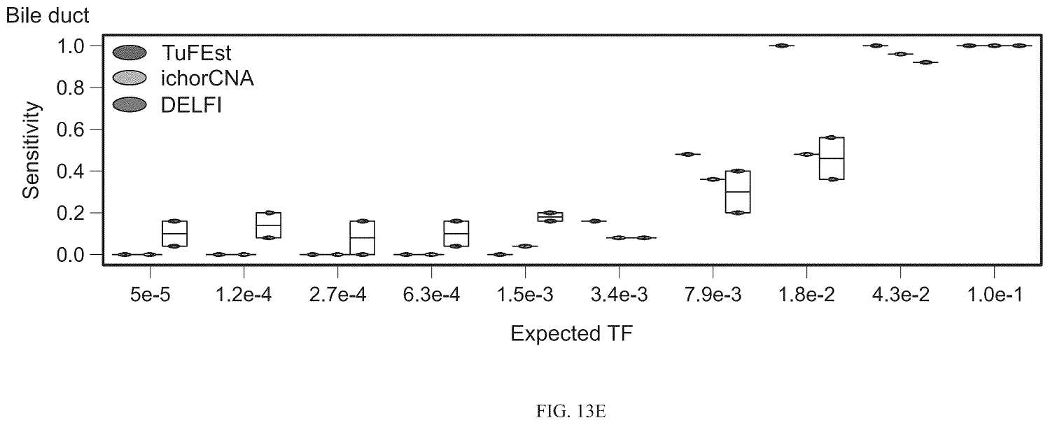

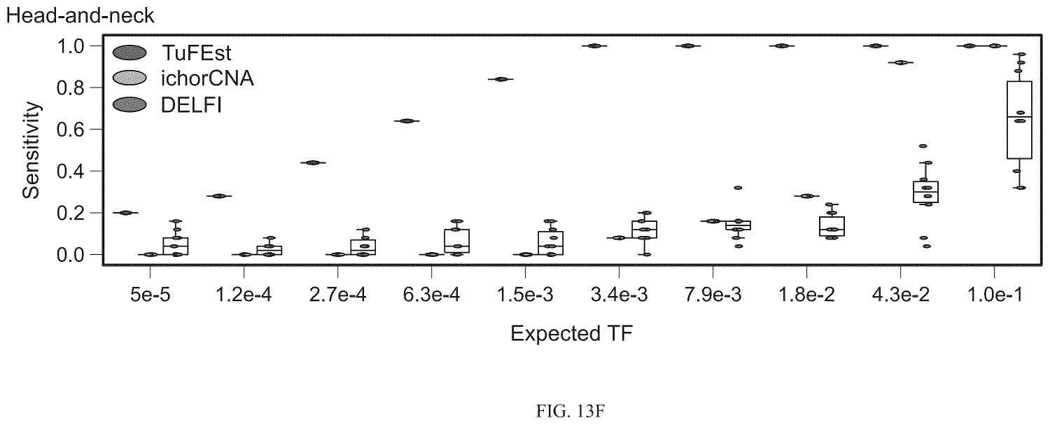

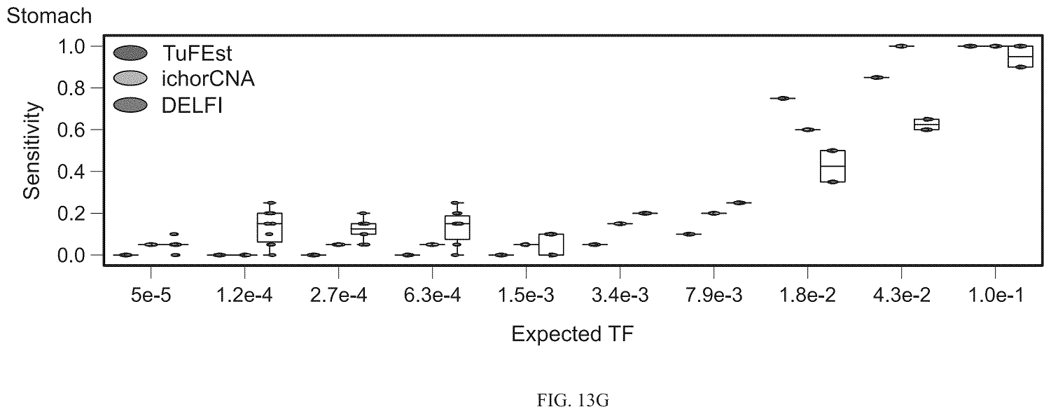

In the above Examples, the methods were trained using separate cohorts of tumor and healthy donor cfDNA data. However, it was hypothesized that the performance of the methods could be further increased by using a patient-matched normal control. Indeed, when evaluating the performance of TuFEst in detecting trace amounts of cancer from serial cfDNA samples where pre-cancer healthy samples from the same person were available, a highly significant gain in the lower limit of detection (LLOD) was observed for all three methods. To evaluate this approach further, the methods were evaluated using data prepared using an in-silico mixing approach to simulate ultra-low pass whole-genome sequencing (ULP-WGS) cell-free DNA (cfDNA) data with very low tumor fraction (TF) (10 TFs logarithmically evenly spaced from 5×10⁻⁵ to 10%), as well as 25 random down-sampled healthy donor data. It was found that at the same FP threshold (e.g., FP=1%), TuFEst achieved similar sensitivity in cancers with at least one order of magnitude lower TF than ichorCNA and DELFI (e.g., median sensitivity ˜80%. TF˜0.3%, 10%, 10%> for TuFEst, DELFI and ichorCNA, respectively) (FIG. 3A). Thus, when evaluating the performance of TuFEst in detecting trace amounts of cancer from serial cfDNA samples when pre-cancer healthy samples from the same person were available, a significant gain in the lower limit of detection (LLOD) by TuFEst among all three methods was observed. TuFEst outperformed both ichorCNA and DELFI in about 90% of testing scenarios across all seven cancer types (FIGS. 13A-13G).

### Example 4: Tumor Fraction Estimator (TuFEst) Detected Cancer Recurrence

For clinical applications, TuFEst's ability to sensitively and accurately detect trace amounts of cancer in serial cfDNA samples can be leveraged to improve cancer detection not only for early screening of cancer but also for monitoring response and resistance to treatment. To formally test TuFEst's ability to detect increasing tumor burden during treatment, it was applied retrospectively to 110 serial blood biopsies from a retrospective cohort of 30 breast cancer patients receiving treatment for advanced breast cancer. Patients were followed clinically, with treatment efficacy and progression defined by standard orthogonal parameters. The cfDNA TF was significantly higher prior to receiving treatments than during the treatment-effective window (FIG. 3B, mean 0.15 vs. 0.056, two-sided Student's t-test P=0.0091), suggesting that TuFEst-estimated TF using ULP-WGS of cfDNA could serve as a proxy for tumor burden and hence a biomarker of treatment efficacy. For example, it was demonstrated that for two patients receiving targeted therapies (FIG. 3C), the TF remained low during the treatment-effective timeline, but it gradually and significantly increased when progression occurred (FIG. 3C, mean 0.056 vs. 0.25, two-sided Student's t-test P=1.4×10⁻⁶). TF was high before the start of a new treatment, while TF remained low when the treatment was still effective, but later on it increased to a high level reflecting cancer relapse possibly due to resistance to treatment. The cfDNA tumor fraction (TF) reflected tumor burden in serial samples. Based on this analysis, it was established that a TuFEst-estimated TF threshold (˜10%) may be used to indicate cancer resistance and signal the potential need to change therapy (FIG. 3B).

In one of the breast cancer patients whose samples were used to test TuFEst (RA_1598), a routine CT scan identified multiple metastases in the liver on day 4,037 as the first clinical evidence of resistance to systemic therapies. TuFEst analysis of the temporal series of samples from this patient revealed that cfDNA TF in all 10 blood biopsies collected from day 3,775 to day 4,026 was consistently higher than 30% (TF mean=46.0%), indicating that TuFEst was able to detect metastatic progression 262 days (˜8 months) earlier than the routine CT scan (FIG. 3D).

Taken together, the above Examples demonstrate the clinical value of TuFEst as a cost-effective, non-invasive method with quick turn-around time to detect cancer progression much earlier than the current standard clinical tests. In addition to its potential as an initial inexpensive pan-cancer screening tool in an asymptomatic population, this earlier detection creates an opportunity to guide clinical decision-making for changes of therapy that could potentially limit or even overcome the development of resistance and therefore improve overall care in cancer patients.

The following methods were employed in the above examples.

### Tu FEst Algorithm

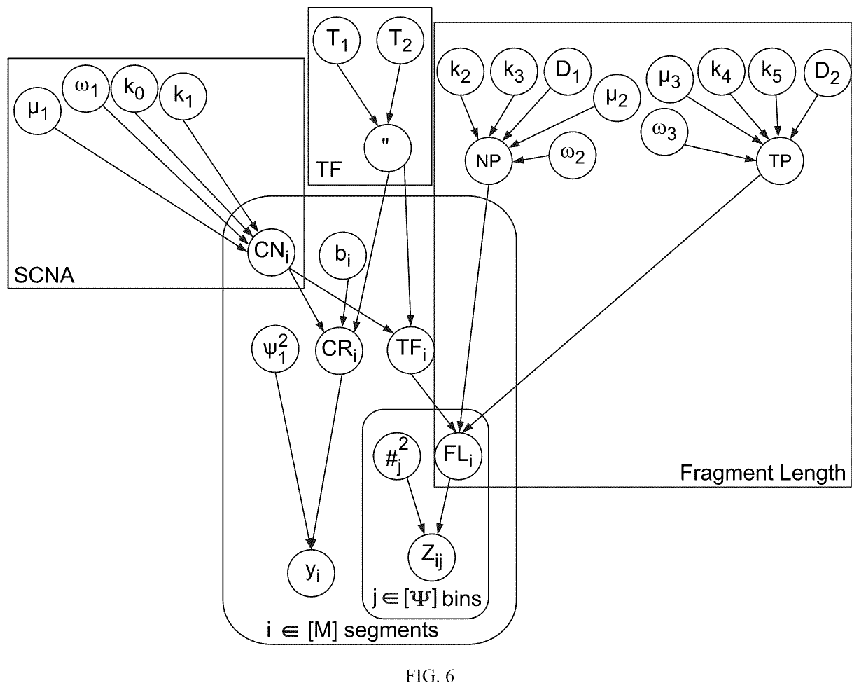

TuFEst used a Bayesian approach, in which the evidence and uncertainties from Y″ fragments and copy number alterations data sources were integrated to produce a joint posterior distribution over tumor fraction (TF) values and predicted total copy-number profile, from which a marginal posterior distribution over the TF values was extracted. TuFEst modeled the cfDNA as a mixture of DNA shed from normal blood cells and an unknown fraction of DNA shed from tumor cells (ctDNA). For each cfDNA sample the tumor fraction (TF), defined as the relative fraction of tumor DNA in the admixture, was estimated by using two different types of tumor-specific aberrations: (i) somatic copy number alterations (SCNAs), and (ii) altered fragment length distribution. Since an increasing number of tumor-specific aberrations improved the sensitivity and accuracy of cancer detection, the 22 pairs of autosomes were split into non-overlapping 5 megabase (Mb) windows and the relative cancer concentration (defined as the log₂(copy ratio)) and the fragment length distribution within each genomic window were calculated. In ULP-WGS, with a depth of ˜0.2×, about 3,000 total fragments per 5 Mb-window were expected. For a given cfDNA ULP-WGS data, TuFEst used a Markov chain Monte Carlo (MCMC) method to sample the joint posterior distribution over the TF values and the predicted total copy-number profile given the observed SCNAs and fragment lengths, from which the marginal posterior distribution over the TF values could be extracted (FIG. 6). The posterior TF values were then used to calculate the expected TF and a 95% confidence interval.

cfDNA Extraction from Whole Blood

Whole blood was collected in EDTA, CellSave, or Streck tubes and processed for plasma extraction utilizing two spins. Blood tubes were centrifuged at 1900×g for 10 minutes and plasma was transferred to a second tube before further centrifugation at 15000×g for 10 minutes. Supernatant plasma was stored at −80° C. until cfDNA extraction. Preferred starting input volume is 6.3 mL plasma, if a sample does not meet this input PBS is added. cfDNA was extracted using the QIAsymphony DSP Circulating DNA Kit according to the manufacturer's instructions. This is a magnetic-particle technology-based chemistry used in conjunction with the QIAsymphony SP instrument manufactured by Qiagen. The cfDNA is bound to magnetic particles. The particle-bound cfDNA is separated from the solution using a covered magnetic rod head. Several wash steps follow to eliminate debris and protein residue from the sample. The machine finishes with a 60 μL cfDNA elution (Qiagen, 2017).

### Library Construction

Initial DNA input was normalized to be within the range of 25-52.5 ng in 50 μL of TE buffer (10 mM Tris HCl ImM EDTA, pH 8.0) according to picogreen quantification. Library preparation was performed using a commercially available kit provided by KAPA Biosystems (KAPA HyperPrep Kit with Library Amplification product KK8504) and IDT's duplex UMI adapters. Unique 8-base dual index sequences embedded within the p5 and p7 primers (purchased from IDT) were added during PCR. Enzymatic clean-ups were performed using Beckman Coultier AMPure XP beads with elution volumes reduced to 30 μL to maximize library concentration.

### Post Library Construction Quantification and Normalization

Library quantification was performed using the Invitrogen Quant-It broad range dsDNA quantification assay kit (Thermo Scientific Catalog: Q33130) with a 1:200 PicoGreen dilution. Following quantification, each library was normalized to a concentration of 35 ng/μL, using Tris-HCl, 10 mM, pH 8.0.

### Library Pool Creation for Ultra-Low Pass Sequencing

In preparation for the sequencing of the ultra-low pass libraries (ULP), approximately, 4 μL of the normalized library was transferred into a new receptacle and further normalized to a concentration of 2 ng/μL using Tris-HCl, 10 mM, pH 8.0. Following normalization, up to 95 ultra-low pass WGS samples were pooled together using equivolume pooling. The pool was quantified via qPCR and normalized to the appropriate concentration to proceed to sequencing.

### Cluster Amplification and Sequencing

Cluster amplification of library pools was performed according to the manufacturer's protocol (Illumina) using Exclusion Amplification cluster chemistry and HiSeqX flowcells. Flowcells were sequenced on v2 Sequencing-by-Synthesis chemistry for HiSeqX flowcells. The flowcells were then analyzed using RTA v.2.7.3 or later. Each pool of ultra-low pass whole genome libraries was run on one lane using paired 151 bp runs.

### WES/WGS Alignment and Quality Control

All DNA sequence data was processed through the Broad Institute's data processing pipeline. For each sample, this pipeline combined data from multiple libraries and flowcell runs into a single BAM file. This file contained reads aligned to the human genome hg 19 genome assembly (version b37) done by the Picard and Genome Analysis Toolkit (GATK) (McKenna, Aaron, Matthew Hanna, Eric Banks, Andrey Sivachenko, Kristian Cibulskis, Andrew Kernytsky, Kiran Garimella, et al. 2010. “The Genome Analysis Toolkit: A MapReduce Framework for Analyzing next-Generation DNA Sequencing Data.” Genome Research 20 (9): 1297-1303) developed at the Broad Institute, a process that involves marking duplicate reads, recalibrating base qualities, and realigning around sINDELs. Reads were aligned to the hg 19 genome assembly (version b37) using BWA-MEM (version 0.7.7-r441).

### Mutation Calling

Prior to variant calling, the impact of oxidative damage (oxoG) to DNA during sequencing was quantified using De ToxoG (Costello, Maura, Trevor J. Pugh, Timothy J. Fennell, Chip Stewart, Lee Lichtenstein, James C. Meldrim, Jennifer L. Fostel, et al. 2013. “Discovery and Characterization of Artifactual Mutations in Deep Coverage Targeted Capture Sequencing Data due to Oxidative DNA Damage during Sample Preparation.” Nucleic Acids Research 41 (6): e67). The cross-sample contamination was measured with ContEst based on the allele fraction of homozygous SNPs (Cibulskis, Kristian, Aaron McKenna, Tim Fennell, Eric Banks, Mark DePristo, and Gad Getz. 2011. “ContEst: Estimating Cross-Contamination of Human Samples in next-Generation Sequencing Data.” Bioinformatics 27 (18): 2601-2), and this measurement was used in the downstream mutation calling pipeline. From the aligned BAM files, somatic alterations were identified using a set of tools developed at the Broad Institute (www.broadinstitute.org/cancer/cga). The details of the sequencing data processing have been described by Berger, Michael F., Michael S. Lawrence, Francesca Demichelis, Yotam Drier, Kristian Cibulskis, Andrey Y. Sivachenko, Andrea Sboner, et al. 2011. “The Genomic Complexity of Primary Human Prostate Cancer.” Nature 470 (7333): 214-20; and by Chapman, Michael A., Michael S. Lawrence, Jonathan J. Keats, Kristian Cibulskis, Carrie Sougnez, Anna C. Schinzel, Christina L. Harview, et al. 2011. “Initial Genome Sequencing and Analysis of Multiple Myeloma.” Nature 471 (7339): 467-72. Briefly, for sSNVs and INDELs detection, high-confidence somatic mutation calls were made by applying MuTect (Cibulskis, Kristian, Michael S. Lawrence, Scott L. Carter, Andrey Sivachenko, David Jaffe, Carrie Sougnez, Stacey Gabriel, Matthew Meyerson, Eric S. Lander, and Gad Getz. 2013. “Sensitive Detection of Somatic Point Mutations in Impure and Heterogeneous Cancer Samples.” Nature Biotechnology 31 (3): 213-19), MuTect2 (Benjamin, D., T. Sato, K. Cibulskis, G. Getz, and C. Stewart. 2019. “Calling Somatic SNVs and Indels with Mutect2.” Biorxiv. biorxiv.org/content/10.1101/861054v1.abstract) and Strelka2 (Kim, Sangtac, Konrad Scheffler, Aaron L. Halpern, Mitchell A. Bekritsky, Eunho Noh, Morten Källberg, Xiaoyu Chen, et al. 2018. “Strelka2: Fast and Accurate Calling of Germline and Somatic Variants.” Nature Methods 15 (8): 591-94) to WES data. Given that normal blood samples might also contain cancer cells, we used DeTiN (Taylor-Weiner, Amaro, Chip Stewart, Thomas Giordano, Mendy Miller, Mara Rosenberg, Alyssa Macbeth, Niall Lennon, et al. 2018. “DeTiN: Overcoming Tumor-in-Normal Contamination.” Nature Methods 15 (7): 531-34) to estimate tumor in normal (TiN) contamination in order to recover falsely rejected sSNVs and sINDELs. Next, four types of filters were applied: (i) a realignment-based filter, which removed variants that could be attributed entirely to ambiguously mapped reads: (ii) an orientation bias filter, which removed possible oxoG and FFPE artifacts (Costello, Maura, Trevor J. Pugh, Timothy J. Fennell, Chip Stewart, Lee Lichtenstein, James C. Meldrim, Jennifer L. Fostel, et al. 2013. “Discovery and Characterization of Artifactual Mutations in Deep Coverage Targeted Capture Sequencing Data due to Oxidative DNA Damage during Sample Preparation.” Nucleic Acids Research 41 (6): e67): (iii) a ContEst filter, which removed variants that might have originated from other samples due to contamination; and (iv) an allele fraction specific panel-of-normals filter, which compared the detected variants to a large panel of normal exomes and removed variants that were observed in several panel-of-normals (PoNs): one consisted of 62 normal samples sequenced using the TWIST bait set: one consisted of 8,334 normal samples from TCGA. All four filters together contributed to the exclusion of potential false-positive events (e.g., commonly occurring germline variants or sequencing artifacts), which ultimately yielded the final list of mutations.

### Copy Number Analysis

For detecting somatic total copy number alterations (sCNAs) the GATK4 CNV pipeline was used (github.com/gatk-workflows/gatk4-somatic-cnvs), which involved the Calculate TargetCoverage, NormalizeSomaticReadCounts, and Circular Binary Segmentation (CBS) algorithms (28. Olshen, Adam B., E. S. Venkatraman, Robert Lucito, and Michael Wigler. 2004. “Circular Binary Segmentation for the Analysis of Array-based DNA Copy Number Data.” Biostatistics 5 (4): 557-72) for genome segmentation.

### Estimation of Tumor Fraction Using WES

To estimate sample tumor fraction using WES data, ABSOLUTE was used, which integrated allele fraction specific information from the sequencing data for sSNVs, INDELs and sCNAs. For each sample, a manual review was conducted to determine the optimal ABSOLUTE (Carter, Scott L., Kristian Cibulskis, Elena Helman, Aaron McKenna, Hui Shen, Travis Zack, Peter W. Laird, et al. 2012. “Absolute Quantification of Somatic DNA Alterations in Human Cancer.” Nature Biotechnology 30 (5): 413-21) solution.

### Definition of Signal-to-Noise Ratio (SNR)

For each cancer cfDNA sample i and a given fragment length bin j, SNR_{i,j} was defined as the fraction of those fragments j in sample i minus the average fraction in a panel of healthy donors, and then divided by the standard deviation of the fraction in the healthy cohort.

### In Silico Admixture and Downsampling Experiments

Two types of in-silico admixture experiments were undertaken. For data included in FIGS. 2B, 2C, 8A-8G, and 9A-9G, for each cancer type, 432 in-silico cancer ULP-WGS data were generated by mixing high TF cfDNA data from cancer patients (TF≥30%, N=14, 3, 7, 8, 6, 3, 3 for prostate, bladder, colon, head-and-neck, bile duct, skin, stomach, respectively) with cfDNA data from 72 independent healthy donors in silico, such that six different TF values were obtained (72 mixes per TF value). To generate 360 healthy donor cfDNA data, each of the 72 healthy donor datasets sequenced to higher coverages (median: 3.5×: range: 1.6-21×) were down-sampled, and 5 ˜0.2× cfDNA data sets were generated to match the depth of the cancer patient samples. Since DELFI requires training, it was trained on cfDNA from 289 cancer patients and 310 of the 360 down-sampled healthy donor cfDNA data and tested it on the in-silico cancer mixtures and the 50 remaining down-sampled healthy donors.

For data included in FIGS. 3A and 13A-13G, for each cancer type, a series of cancer cfDNA ULP-WGS of ultra-low TF (for ten TF logarithmically evenly spaced from 5×10⁻⁵ to 10%) was generated using multiple high TF cancer cfDNA (TF≥65%, N=5, for breast: TF≥15%, N=5, 5, 5, 5, 5, 5, 4 for prostate, bladder, colon, head-and-neck, bile duct, skin, stomach, respectively) and one healthy donor. For each TF in each cancer type, 5 different samples (˜0.2× sequencing depth) for each pair of different cancers and the same healthy donor were generated using different random seeds. This set was labeled as “cancer” in the analysis. This simulated a new paradigm in which access to pre-cancerous plasma samples from each participant was available from when he/she was still healthy, for example, through routine physicals. Twenty five (25) different samples with matching depth (˜0.2× sequencing depth) were generated from the same healthy donor using different random seeds and the set was labeled as “healthy” in the analysis. Since DELFI required training, it was trained on cfDNA from 289 cancer patients and 355 of the 360 down-sampled healthy donor cfDNA data, and it was tested on the in-silico cancer mixtures (N=25, 25, 25, 25, 25, 25 for prostate, bladder, colon, head-and-neck, bile duct, skin, respectively) and the 25 down-sampled data from the same healthy donor.

Implementation of DELFI and ichorCNA

The implementation of DELFI used the codes included in Cristiano, Stephen, Alessandro Leal, Jillian Phallen, Jacob Fiksel, Vilmos Adleff, Daniel C. Bruhm, Sarah Østrup Jensen, et al. 2019. “Genome-Wide Cell-Free DNA Fragmentation in Patients with Cancer.” Nature 570 (7761): 385-89. For the data included in FIGS. 2B, 2C, 8A-8G, and 9A-9G, for each TF in each cancer type, the training set included ULP-WGS of 289 real cancer cfDNA and 310 healthy cfDNA data generated from 62 healthy donors (62×5=310), while the testing set included the respective 72 in-silico mixture cancer cfDNA (for the particular cancer type and TF value) and 50 healthy cfDNA derived from 10 independent healthy donors (10×5=50). To ensure that the results did not depend on the choice of the healthy donors used for training vs. testing, random splits were done to train and test 10 times. For data included in FIGS. 3A and 13A-13G, for each TF in each cancer type, the training set included ULP-WGS of 289 real cancer cfDNA and 355 healthy cfDNA data generated from 71 healthy donors (71×5=355) and left the one healthy sample used for generating in-silico mixtures and the downsampled healthy set out, while the testing set included the N(=25, 25, 25, 25, 25, 25, 25 for prostate, bladder, colon, head-and-neck, bile duct, skin respectively) in-silico cancer mixtures and 25 down-sampled data from the same left-out healthy donor. Each cancer type was randomly paired with a different healthy donor out of all the 72 possible choices. To report the distribution of results, 80% of the original testing set was randomly downsampled 10 times.

The ichorCNA (Adalsteinsson, Viktor A., Gavin Ha, Samuel S. Freeman, Atish D. Choudhury, Daniel G. Stover, Heather A. Parsons, Gregory Gydush, et al. 2017. “Scalable Whole-Exome Sequencing of Cell-Free DNA Reveals High Concordance with Metastatic Tumors.” Nature Communications 8 (1): 1324) was run the same (in-silico) cancer and healthy samples, with default settings.

Classification of cfDNA Samples During Treatment (N_Pat=30, N_Samples=110)

cfDNA samples during clinical treatment were classified into three different groups given their collection dates relative to the received treatments and disease progression status: (1). For cfDNA collected before receiving any treatment, samples were classified as “Pre-treatment” (N=6): (2). For cfDNA collected within a treatment window with duration ≥180 days, and collected >3 days after the start date, >10 days before the end date, they were classified as “On-treatment” (N=30): (3). For cfDNA collected within <10 days before the end date due to disease progression (treatment duration ≥180 days), or in the intervals between a failed and not yet receiving a new treatment, they were classified as “End- or post-therapy” (N=38).

### Data Preprocessing

The ultra-low-pass (ULP) whole genome sequencing (WGS) data (i.e., BAM file) were first divided into B′ (566) non-overlapping bins of size S (5 Mb) across autosomes (i.e., chr1:1-S, chr1: (S+1)-2S . . . ). The total number of aligned reads and their fragment length distribution were calculated for the reads within each bin (using GATK4 CollectReadCounts for the total number of reads, and pysam library for fragment length distribution). For calculating the fragment length distribution, only read pairs with high mapping quality (i.e., MAPQ≥q: q=30) and an insert size between 1 and T (1000 bp) were used. PCR or optical duplicates are removed. Bins that overlapped genomic regions that were undefined (i.e., all “N”s) were removed. Similarly, bins at the end of the chromosome arms that were smaller than the others were also removed, yielding B (B=490) actual bins.

To create a normal reference data set, WGS on cfDNA from H (H=20) healthy donors and was performed and the data was analyzed with the same pipeline. The reference data set was used to normalize the coverage at each bin, accounting for the biases generated by the library construction, the sequencing platform and cfDNA-specific artifacts, using the Tangent normalization method (Tabak et al., The Tangent copy-number inference pipeline for cancer genome analyses, doi: doi.org/10.1101/566505): for each healthy donor h∈{1, 2, . . . , H} and each bin b∈{1,2, . . . , B}, the total number of aligned reads in bin b, ie., ch b, was first determined, from which the log₂ fraction of reads that fall in the bin was calculated, i.e.,

$${{f}^{h}}_{b} ( = {log}_{2} ( \frac{{{c}^{h}}_{b}}{{\sum }_{b = 1}^{B} {{c}^{h}}_{b}} ) ) .$$

The collection of log₂ fractions was described as a vector, f^{h}=[f^{h}₁, f^{h}₂, . . . , f^{h}_{B}]. The entire dataset from the H healthy donors constituted the Panel-of-Normals (PON), i.e. f^{href}={f^{h}}^{H}ₕ₌₁.

For a cfDNA sample from a cancer patient, t, the same procedure was followed to generate f^{t}=[f^{t}₁, f^{t}₂, . . . , f^{t}_{B}]. Next, tangent normalization was performed for this sample using the created PoN to get the log₂-transformed copy ratio across the genome, i.e., l^{t}=f^{t}−f^{href,t}, where f^{href,t} represents the projection of f^{t} into the linear subspace spanned by the PON (f^{href}). Finally, circular binary segmentation (CBS) was performed on l^{t} to identify genomic segments (the bins within the same segment with the same total copy number) across the genome. M^{t} represented the number of genomic segments for the sample t. Note that all of the algorithms mentioned above were implemented as individual modules in the GATK3 suite, and they were integrated in a single workflow (zlin/gatk_acnv_wgs) consisting of tangent normalization (GATK3 NormalizeSomaticReadCounts) and CBS segmentation (GATK3 PerformSegmentation, CallSegments).

For each sample k (either from a healthy donor or cancer patient), the fragment length distribution of cfDNA fragments with size r between 1-T bp (T=1000) in each bin b E {1, 2, . . . , B} was also calculated. F^{k}_{r,b} represented the fraction of DNA fragments with length r in the genomic segment b for sample k. Also, by integrating all high quality fragments across the genome, a sample-level fragment length distribution, which we denote as F^{t}ᵣ was also calculated for the cancer patient t and F^{h}ᵣ for the healthy donor h.

### Feature Selection

In order to select the cfDNA fragments with enriched tumor signals, significance metrics were designed that quantify the cancer signals relative to the noise (where the noise can represent variability across the healthy population, sequencing experimental conditions, etc.):
1) Signal-to-Noise Ratio (SNR): for a given tumor sample t, across all cfDNA fragment lengths r, the signal-to-noise ratio was calculated: SNRᵣ=(F^{t}ᵣ−F̅^{H}ᵣ)/std(F^{H}ᵣ), where F̅^{H}ᵣ,represents the average over the healthy panel of normals (PoN) of the fraction of cfDNA fragments with length r, i.e.,

$${{\overline{F}}^{H}}_{r} = \frac{{\sum }_{h = 1}^{H} {{F}^{H}}_{r}}{H}$$

and std(F^{H}ᵣ) represents their standard deviation, i.e.,

$$std ( {{F}^{H}}_{r} ) = \sqrt{\frac{{\sum }_{h = 1}^{H} {( {{F}^{H}}_{r} - {{\overline{F}}^{H}}_{r} )}^{2}}{H}} .$$

A high SNR was expected for fragment lengths that carried increased cancer signals.
2) Spearman correlation coefficient between the log₂ (copy ratio) and fragment length distribution: for a given cancer sample t and fragment length r, the Spearman correlation coefficient between the log₂-transformed copy ratio and the fraction of fragments with length r across the genomic segments with the most extreme copy number alterations (top 10% for amplifications or bottom 10% for deletions) was calculated. A high Spearman correlation was expected for fragment lengths enriched with cancer signals.

Based on the data, it was found that fragments with sizes between 261 bp and 310 bp generally contained the highest cancer signals across various cancer types. Therefore, signals from 261-310 bp were incorporated in the TuFEst model.

### Tu FEst Algorithm: Tumor Fraction Estimation in Cell-Free DNA

cfDNA from cancer patients can be modeled as a two-component mixture that includes DNA fragments from cancer and normal cells. TuFEst used a Bayesian model to infer the underlying tumor fraction and the total copy number profile in cancer cells simultaneously by leveraging the observed cancer-specific signals, including copy number alterations and altered fragment length distribution. To illustrate this idea, for a given cfDNA sample, let α represent the tumor fraction, CNᵢ represent the total copy number of the i-th genomic segment in the cancer cells, bⱼ represent the length of the i-th segment, M represent the total number of genomic segments, NPⱼ represent the fraction of fragments (with length j) in healthy donors inferred from the panel of normals (PoN) (called Normal ‘pole’), TPⱼ represent the fraction of cancer cells-derived fragments (with length j) inferred from cfDNA samples with high tumor fraction (called tumor ‘pole’). It is important to note that a good PON should match closely to the tested cfDNA sample in terms of experimental conditions including sample collection, cfDNA library preparation, sequencing platforms, etc., to avoid possible batch effects.

### Define

$$γ = \frac{{\sum }_{i = 1}^{M} {b}_{i} \times {CN}_{i}}{{\sum }_{i = 1}^{M} {b}_{i}}$$

$${CR}_{i} = [ α \times {CN}_{i} + 2 \times ( 1 - α ) ] / [ α \times γ + 2 \times ( 1 - α ) ] , i ∈ \{ 1 , 2 , 3 , … , M \}$$

where γ is the normalized copy number across the genome in cancer cells (known as ploidy), and CRᵢ represents the expected copy ratio of the i-th segment. Also, by definition, for each segment i

$${TF}_{i} = \frac{α \times {CN}_{i}}{α \times {CN}_{i} + ( 1 - α ) \times 2} , i ∈ \{ 1 , 2 , 3 , … , M \}$$

where TFᵢ represents the “local” tumor fraction of the i-th segment, and the following is calculated

$${FL}_{i , j} = ( {NP}_{j} \times ( 1 - {TF}_{i} ) + {TP}_{j} \times {TF}_{i} ) \times \frac{{CR}_{i}}{B} , \\ i ∈ \{ 1 , 2 , 3 , … , M \} , j ∈ [ 261 , 310 ]$$

where FL_{i,j} is the expected fraction of fragments (with length j) in the i-th genomic segment.

### Emission Model

For each segment i, given the expected copy ratio CRᵢ, the observed copy ratio averaged across all the genomic bins (of size S) within segment i, i.e. yᵢ, was modeled using a log-Normal distribution with CRᵢ and ψᵢ²/Xᵢ as the mean and variance parameters respectively, where ψᵢ² is the variance of observed copy ratio across all genomic bins (of size S) within segment i, and Xᵢ is the number of bins in segment i. If there was only one genomic bin in segment i, then ψᵢ² was set using a default value ψ₀², ie. ψᵢ²=ψ₀². Therefore, yᵢ˜logNormal(CRᵢ, ψᵢ²/Xᵢ), i∈{1,2,3, . . . , M}, j∈[261,310].

Next, given the expected fraction of cfDNA fragments with length j in segment i, FL_{i,j}, the observed fraction of cfDNA fragments with length j averaged across all the genomic bins within segment i, i.e. z_{i,j}, was modeled using a Normal distribution with FL_{i,j}, δ_{i,j}²/Xᵢ as the mean and variance parameters respectively, where δ_{i,j}² is the variance of observed fraction of cfDNA fragments with length j across all genomic bins within segment i, and Xᵢ is the number of bins in segment i. If there was only one genomic bin in segment i, then δ_{i,j}²=δ_{0,j}². Therefore, zᵢⱼ˜Normal(FL_{i,j}, δ_{i,j}²/Xᵢ), i∈{1,2,3, . . . , M}, j∈[261,310].

Even though cfDNA fragments with sizes between 261-310 bp were used in the TuFEst model described in the Examples, it is important to point out that the methodology can be easily generalized to include other fragments with different sizes based on parameters learned from the specific dataset.

Moreover, the relative weight of log-likelihood between copy ratio and fragment length is also a flexible parameter called cn_weight. For example, if cn_weight=10, the log-likelihood of the copy ratios is weighted 10 times more than that of fragment length log-likelihood (the default is 10).

### Prior Model

Priors were assigned for the following parameters in the generative model: CNᵢ, α, NP, TP, with hyperparameters μ_{1,2,3}, ω_{1,2,3}, τ_{1,2}, D_{1,2}.

$${CN}_{i} ∼ Truncated Normal ( {μ}_{1} , {{ω}_{1}}^{2} , L = {k}_{0} , U = {k}_{1} ) , i ∈ \{ 1 , 2 , 3 , … , M \} ,$$

$$α ∼ Beta ( {τ}_{1} , {τ}_{2} )$$

$${NP}_{j} ∼ Truncated Normal ( {μ}_{2} , {{ω}_{2}}^{2} , L = {k}_{2} , U = {k}_{3} ) \times {D}_{1 , j} , j ∈ [ 261 , 310 ]$$

$${TP}_{j} ∼ Truncated Normal ( {μ}_{3} , {{ω}_{3}}^{2} , L = {k}_{4} , U = {k}_{5} ) \times {D}_{2 , j} , j ∈ [ 261 , 310 ]$$

where D₁ is a rough reference fragment length distribution based on the PON, k₂,k₃, μ₂, ω₂² together represent the fluctuation across healthy individuals in the panel of normals (PoN), D₂ is a rough reference fragment length distribution for the tumor ‘pole’, and k₄, k₅, μ₃, ω₃² together represent the fluctuation of the tumor ‘pole’. The default values of these hyper-parameters are shown in Table 1 below.

### Learning and Inference

The joint posterior distribution of the parameters underlying the generative model (α, NP, TP, CN_{i∈[NB]}) was inferred using a Markov chain Monte Carlo (MCMC) method, and the marginal posterior distribution of α was used to quantify the tumor fraction as well as its uncertainty in the given sample. Note that due to empirical non-linear effects, in order to enhance the power to distinguish between trace amount of cancer (low α) and no cancer (α=0), when the posterior mean of α was less than 10% (i.e., α̅<10%), a slightly different set of normal and tumor poles was used and the MCMC was then rerun. The updated tumor and normal poles were:

$${NP}_{j} ∼ Truncated Normal ( {{μ}^{′}}_{2} , {{{ω}^{′}}_{2}}^{2} , L = {{k}^{′}}_{2} , U = {{k}^{′}}_{3} ) \times {{D}^{′}}_{1 , j} , j ∈ [ 261 , 310 ]$$

$${TP}_{j} ∼ Truncated Normal ( {{μ}^{′}}_{3} , {{{ω}^{′}}_{3}}^{2} , L = {{k}^{′}}_{4} , U = {{k}^{′}}_{5} ) \times {{D}^{′}}_{2 , j} , j ∈ [ 261 , 310 ]$$

$$cn\_weight ’ = 10$$

Then, the posterior was interpolated by mixing the two MCMC runs based on the fraction of healthy donors that had expected tumor fraction less than α̅ from the second MCMC. For example, if 80% of healthy donors had expected tumor fraction less than α̅, then the first chain was mixed with the second chain in a ratio of 80%: 20%.

TABLE 1
Default parameters for the TuFEst algorithm

| | | Default |
|---|---|---|
| Parameter | Interpretation | value |
| S | Size of a genomic bin | 5 |
| | (in Megabase) | |
| B′ | Number of genomic bins | 566 |
| | across the autosomes | |
| B | Eventual number of bins | 490 |
| q | Mapping quality threshold for | 30 |
| | the fragments to be included | |
| hgX | Version of UCSC reference | hg19 |
| | genome | |
| L | Lower limit parameter in the | — |
| | truncated normal distribution | |
| U | Upper limit parameter in the | — |
| | truncated normal distribution | |
| k₀ | Lower bound of the truncated | 0.01 |
| | normal distribution for total | |
| | copy number | |
| k₁ | Upper bound of the truncated | 9.99 |
| | normal distribution for total | |
| | copy number | |
| μ₁ | Mean of the truncated normal | 2.0 |
| | distribution for total copy | |
| | number | |
| ω₁ | Standard deviation of the | 1.0 |
| | truncated normal distribution | |
| | for total copy number | |
| k₂ | Lower bound of the truncated | 1.0 |
| | normal distribution for the | |
| | normal reference pole | |
| k₃ | Upper bound of the truncated | 4.0 |
| | normal distribution for the | |
| | normal reference pole | |
| μ₂ | Mean of the truncated normal | 1.3 |
| | distribution for the normal | |
| | reference pole | |
| ω₂ | Standard deviation of the | 0.1 |
| | truncated normal distribution | |
| | for the normal reference pole | |
| k₄ | Lower bound of the truncated | 0.5 |
| | normal distribution for the | |
| | imaginary tumor pole | |
| k₅ | Upper bound of the truncated | 6.0 |
| | normal distribution for the | |
| | imaginary tumor pole | |
| μ₃ | Mean of the truncated normal | 3.0 |
| | distribution for the imaginary | |
| | tumor pole | |
| ω₃ | Standard deviation of the | 0.05 |
| | truncated normal distribution | |
| | for the imaginary tumor pole | |
| D_{1,261-270bp} | Reference fraction of | 0.0010 |
| | fragments (261-270 bp) for | |
| | the normal pole | |
| D_{1,271-280bp} | Reference fraction of | 0.0010 |
| | fragments (271-280 bp) for | |
| | the normal pole | |
| D_{1,281-290bp} | Reference fraction of | 0.0013 |
| | fragments (281-290 bp) for | |
| | the normal pole | |
| D_{1,291-300bp} | Reference fraction of | 0.0019 |
| | fragments (291-300 bp) for | |
| | the normal pole | |
| D_{1,301-310bp} | Reference fraction of | 0.0027 |
| | fragments (301-310 bp) for | |
| | the normal pole | |
| D_{2,261-270bp} | Reference fraction of | 0.0074 |
| | fragments (261-270 bp) for | |
| | the tumor pole | |
| D_{2,271-280bp} | Reference fraction of | 0.0093 |
| | fragments (271-280 bp) for | |
| | the tumor pole | |
| D_{2,281-290bp} | Reference fraction of | 0.013 |
| | fragments (281-290 bp) for | |
| | the tumor pole | |
| D_{2,291-300bp} | Reference fraction of | 0.017 |
| | fragments (291-300 bp) for | |
| | the tumor pole | |
| D_{2,301-310bp} | Reference fraction of | 0.022 |
| | fragments (301-310 bp) for | |
| | the tumor pole | |
| τ₁ | Alpha parameter of the Beta | 2 |
| | distribution | |
| τ₂ | Beta parameter of the Beta | 5 |
| | distribution | |
| ψ²₀ | Variance of copy ratio | 0.0006 |
| δ_{0,261-270bp}² | Variance of fraction of | 2.98e−12 |
| | fragments (261-270 bp) | |
| δ_{0,271-280bp}² | Variance of fraction of | 3.75e−12 |
| | fragments (271-280 bp) | |
| δ_{0,281-290bp}² | Variance of fraction of | 6.46e−12 |
| | fragments (281-290 bp) | |
| δ_{0,291-300bp}² | Variance of fraction of | 1.04e−11 |
| | fragments (291-300 bp) | |
| δ_{0,301-310bp}² | Variance of fraction of | 2.00e−11 |
| | fragments (301-310 bp) | |
| cn_weight | The times that log-likelihood | 1 |
| | favors for copy ratio | |
| Xᵢ | The bins in segment i from | — |
| | the output of the segmentation | |
| | algorithm | |
| k′₂ | Lower bound of the truncated | 2.0 |
| | normal distribution for the | |
| | second normal reference pole | |
| k′₃ | Upper bound of the truncated | 4.0 |
| | normal distribution for the | |
| | second normal reference pole | |
| μ′₂ | Mean of the truncated normal | 2.5 |
| | distribution for the second | |
| | normal reference pole | |
| ω′₂ | Standard deviation of the | 0.1 |
| | truncated normal distribution | |
| | for the second normal | |
| | reference pole | |
| k′₄ | Lower bound of the truncated | 1.0 |
| | normal distribution for the | |
| | second imaginary tumor pole | |
| k′₅ | Upper bound of the truncated | 6.0 |
| | normal distribution for the | |
| | second imaginary tumor pole | |
| μ′₃ | Mean of the truncated normal | 3.0 |
| | distribution for the second | |
| | imaginary tumor pole | |
| ω′₃ | Standard deviation of the | 0.05 |
| | truncated normal distribution | |
| | for the second imaginary | |
| | tumor pole | |
| D′_{1,261-270bp} | Reference fraction of | 0.0018 |
| | fragments (261-270 bp) for | |
| | the second normal pole | |
| D′_{1,271-280bp} | Reference fraction of | 0.0022 |
| | fragments (271-280 bp) for | |
| | the second normal pole | |
| D′_{1,281-290bp} | Reference fraction of | 0.0031 |
| | fragments (281-290 bp) for | |
| | the second normal pole | |
| D′_{1,291-300bp} | Reference fraction of | 0.0049 |
| | fragments (291-300 bp) for | |
| | the second normal pole | |
| D′_{1,301-310bp} | Reference fraction of | 0.0076 |
| | fragments (301-310 bp) for | |
| | the second normal pole | |
| D′_{2,261-270bp} | Reference fraction of | 0.0074 |
| | fragments (261-270 bp) for | |
| | the second tumor pole | |
| D′_{2,271-280bp} | Reference fraction of | 0.0093 |
| | fragments (271-280 bp) for | |
| | the second tumor pole | |
| D′_{2,281-290bp} | Reference fraction of | 0.013 |
| | fragments (281-290 bp) for | |
| | the second tumor pole | |
| D′_{2,291-300bp} | Reference fraction of | 0.017 |
| | fragments (291-300 bp) for | |
| | the second tumor pole | |
| D′_{2,301-310bp} | Reference fraction of | 0.022 |
| | fragments (301-310 bp) for | |
| | the second tumor pole | |

TABLE 2
Observed variables

| | |
|---|---|
| f^{h}_{b} | Fraction of aligned reads that fall in the |
| | bin b in the healthy donor h |
| f^{t}_{b} | Fraction of aligned reads that fall in the |
| | bin b in the cancer sample t |
| F^{h}ᵣ | Fraction of fragments with size r in the |
| | healthy donor h |
| F^{t}ᵣ | Fraction of fragments with size r in the |
| | cancer sample t |
| log₂(cr^{t}_{b}) | Tangent normalized log2(copy ratio) in the |
| | bin b in the cancer sample t |
| F^{t}_{r,b} | Fraction of reads with size r in the bin b in |
| | the cancer sample t |

### OTHER EMBODIMENTS

From the foregoing description, it will be apparent that variations and modifications may be made to the disclosure described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated herein by reference to the same extent as if each independent patent and publication was specifically and individually indicated to be incorporated herein by reference. The application may relate to PCT Application No. PCT/US2019/032914, filed May 17, 2019, or to PCT Application No. PCT/US2017/022792, filed Mar. 16, 2017, the disclosures of each of which are incorporated herein by reference in their entireties for all purposes.

What is claimed is:

1. A method for characterizing DNA in a biological sample from a subject having or suspected of having a neoplasia or for detecting resistance to therapy in a subject being treated for a neoplasia, the method comprising:
(a) sequencing cell free DNA (cfDNA) derived from a biological sample to obtain sequence data:
(b) analyzing the sequence data to determine a copy number profile and DNA fragment length abundance profile; and
(c) calculating a tumor fraction in the cfDNA based upon the copy number profile and the fragment length abundance profile, thereby characterizing the DNA in the biological sample:
(a) sequencing cell free DNA (cfDNA) derived from a biological sample to obtain sequence data:
(b) analyzing the sequence data to calculate a copy number profile and DNA fragment length abundance profile, wherein said fragment length abundance profile has a signal-to-noise ratio (SNR) of at least 2 and an absolute correlation coefficient of at least 0.1 with log₂ transformed copy ratios associated with a neoplasia; and
(c) using a probabilistic model combining the copy number profile and the DNA fragment length abundance profile to calculate tumor fraction in the cfDNA, thereby characterizing the DNA in the biological sample:
(a) sequencing cell free DNA (cfDNA) derived from a biological sample to obtain sequence data:
(b) determining in the sequence data the DNA fragment length abundance profile for DNA fragments with lengths of from about 261 to about 310 bp; and
(c) using a probabilistic model to calculate tumor fraction in the cfDNA based upon the DNA fragment length abundance profile, wherein a non-zero tumor fraction indicates that the subject has a neoplasia; or
(a) sequencing cell free DNA (cfDNA) derived from a biological sample derived from the subject to obtain sequence data;
(b) analyzing the sequence data to determine a copy number profile and DNA fragment length abundance profile; and
(c) calculating a tumor fraction in the cfDNA based upon the copy number profile and the fragment length abundance profile, wherein the method identifies the presence or absence of a neoplasia in the biological sample or wherein a significant increase in tumor fraction over time and/or a tumor fraction above a threshold value detects resistance.

2. The method of claim 1, wherein the DNA fragment length abundance profile comprises a signal-to-noise ratio (SNR) of at least 2 and an absolute correlation coefficient of at least 0.1 with log₂ transformed copy ratios associated with a neoplasia.

3. The method of claim 1, wherein the fragment length abundance profile is calculated for fragment lengths between about 100 and about 500 base pairs, between about 100 and about 400 base pairs, between about 200 and about 400 base pairs, or between about 261 and about 310 base pairs.

4. The method of claim 1, wherein the SNR is calculated across contiguous fragment-length bins within a range of fragment lengths for which the fragment length abundance profile is calculated; or
is calculated as SNR_{i,j}, wherein i is a cell free DNA sample, j is a bin of fragment lengths, and SNR_{i,j} is the fraction of those fragments j in sample i minus the average fraction in a panel of healthy donors, and then divided by the standard deviation of the fraction in the panel of healthy donors.

5. The method of claim 4, wherein the SNR is a maximum SNR calculated in a bin within a fragment-length range for which the DNA fragment length abundance profile is calculated.

6. The method of claim 4, wherein the bin is 5 bp, 10 bp, 15 bp, or 20 bp in size.

7. The method of claim 1, wherein the SNR is calculated as SNRᵣ=(F^{t}ᵣ−F̅^{H}ᵣ)/std(F^{H}ᵣ), wherein F^{t}ᵣ represents DNA fragment length bin r in biological sample t, and F̅^{H}ᵣ represents the average over a healthy panel of normals of the fraction of DNA fragments in fragment length bin r.

8. The method of claim 1, wherein the correlation coefficient is a Spearman Correlation Coefficient.

9. The method of claim 1, wherein the tumor fraction is less than about 0.03; is from about 1e-4 to about 0.03; is from about 5e-3 to about 0.15; or is between about 1e-5 and about 0.1.

10. The method of claim 1, further comprising comparing the copy number profile and the fragment length abundance profile to a matched normal sample.

11. The method of claim 1, wherein the neoplasia is selected from the group consisting of bile duct cancer, bladder cancer, breast cancer, colon cancer, head-and-neck cancer, liver cancer, lung cancer, intrahepatic bile duct cancer, prostate, ovarian cancer, skin cancer, stomach cancer, thyroid, and chronic lymphocytic leukemia (Richter's transformation).

12. The method of claim 1, wherein the sequencing is next generation sequencing or ultra low-pass whole genome sequencing.

13. The method of claim 1, wherein the calculating is done on a computer system.

14. A method for monitoring therapy in a subject being treated for a neoplasia, the method comprising:
(a) sequencing cell free DNA (cfDNA) derived from two or more biological samples derived from the subject to obtain sequence data, wherein the two or more biological samples are obtained at one or more time points during a course of treatment:
(b) analyzing the sequence data to determine a copy number profile and a DNA fragment length abundance profile; and
(c) calculating a tumor fraction in the cfDNA based upon the copy number profile and the fragment length abundance profile, thereby monitoring the therapy.

15. The method of claim 14, further comprising collecting biological samples from the subject about once per day, every 3 days, every 1 week, 2 weeks, 3 weeks, or month and determining a tumor fraction in the cfDNA of each biological sample.

16. The method of claim 14, wherein the copy number profile and/or the DNA fragment length abundance profile is calculated over 1, 2, 3, 4, 5, or all genomic loci represented in the sequence data.

17. A computer-implemented method comprising:
receiving sequencing data from a plurality of cfDNA obtained from a plurality of biological samples:
defining, for a plurality of cfDNA present in a biological sample, a copy number profile and a fragment length abundance profile, wherein the copy number profile comprises a copy ratio of a plurality of somatic copy number alterations (SCNA), and wherein the fragment length abundance profile comprises one or more of a plurality of aligned reads and an associated fragment length distribution for non-overlapping bins of the sequencing data:
determining whether a Signal-to-noise Ratio (SNR) across the fragment length abundance profile and a correlation coefficient of the copy ratio and a fraction of fragments associated with a neoplasia satisfy one or more criteria; and
calculating a tumor fraction (TF) of the biological sample based on at least one of the fragment length abundance profile for which the SNR satisfies the one or more criteria and the copy ratio and the fraction of fragments for which the correlation coefficient satisfies the one or more criteria; or
sequencing polynucleotide data from a plurality of biological samples:
identifying a copy ratio of a plurality of somatic copy number alterations (SCNA) and an associated fragment length distribution for non-overlapping bins of the sequencing data:
determining whether a Signal-to-noise Ratio (SNR) across the fragment length distribution and a correlation coefficient of the copy ratio and the fragment length distribution associated with a neoplasia satisfy one or more criteria; and
calculating a tumor fraction (TF) profile of the biological sample based on at least one of a size of a genomic bin and a number of genomic bins of the sequencing data; and
determining whether the polynucleotide data came from cancer cells based on the fragment length distribution for which the SNR satisfies the one or more criteria, a copy ratio for which the correlation coefficient satisfies the one or more criteria, and the TF profile.

18. The computer-implemented method of claim 17, wherein the TF profile is calculated based on one or more of a total copy number of a genomic bin in the cancer cells, a length of the genomic bin, a total number of genomic bins, a fraction of fragments in healthy donors inferred from a panel of normals (PoN), and a fraction of cancer cells-derived fragments inferred from cfDNA samples with a high tumor fraction.

19. The method of claim 18, wherein the biological sample comprises a liquid sample or a solid sample.

20. The method of claim 18, wherein the biological sample comprises a bodily fluid or a tissue sample.
